(19) United States
(12) Patent Application Publication
(10) Pub. No.: US 2020/0170291 A1
(43) Pub. Date: Jun. 4, 2020

(54) PROCESSES FOR PRODUCING LACTITOL GRANULATES AND GRANULATES PRODUCED THEREBY

(71) Applicant: PARTICLE DYNAMICS INTERNATIONAL, LLC

(72) Inventors: Keith Cromack, St. Louis, MO (US); Steve Freebersyser, St. Louis, MO (US); Paul Brady, St. Louis, MO (US)

(73) Assignee: PARTICLE DYNAMICS INTERNATIONAL, LLC, St. Louis, MO (US)

(21) Appl. No.: 16/615,455

(22) PCT Filed: May 22, 2018
(86) PCT No.: PCT/US2018/033918

§ 371 Date: Nov. 21, 2019

Related U.S. Application Data
(60) Provisional application No. 62/509,966, filed on May 23, 2017.

(51) Int. Cl. A23P 10/20; A23L 27/30; A23L 33/125; A61K 9/16

(52) U.S. Cl. CPC A23P 10/20; A61K 9/16; A23L 33/125; A23L 27/34

(57) ABSTRACT

The present invention is generally directed to processes for producing lactitol granulates. The present invention is also directed to lactitol granulates exhibiting one or more desirable and improved properties as compared to prior lactitol granulates.

### FIELD OF THE INVENTION

The present invention is generally directed to processes for producing lactitol granulates. The present invention is also directed to lactitol granulates exhibiting one or more desirable and improved properties as compared to prior lactitol granulates.

### BACKGROUND OF THE INVENTION

Lactitol (4-O-α-D-Galactopyranosyl-D-glucitol) is known for use as a sweetener that can be used as a total or partial replacement for sucrose. Lactitol does not cause increased blood glucose content and is non-cariogenic and therefore advantageous from a dental perspective. The preparation of lactitol is well-known in the art and generally occurs by hydrogenation of lactose in the presence of a Raney nickel catalyst.

Crystalline lactitol is known to exist in the anhydrous, monohydrate, dihydrate, trihydrate, and hexahydrate forms. Many lactitol products include lactitol in the form of the crystalline monohydrate form, which is defined by standards such as the European Union National Pharmacopoeia (EU-NP) and USP National Formulary (USP-NF) standard defining lactitol monohydrate as having moisture content (free+bound water) of from 4.5-5.5 wt %. Amorphous lactitol products are known as well.

Solid, particulate lactitol products—including crystalline and/or amorphous lactitol—are known in the art. Generally, these products are incorporated into a variety of edible and pharmaceutical compositions. For example, they are known as suitable for use as a directly compressible powder for use in preparing lactitol-containing tablets.

A need exists in the art for a solid lactitol product (e.g., a lactitol granulate) that may be utilized as a composition for administration itself that does not require any other excipients.

### BRIEF SUMMARY OF THE INVENTION

Briefly, therefore, the present invention is generally directed to processes for preparing lactitol granulates and improved lactitol granulates. Granulates of the present invention are suitable for use and administration as the composition itself. That is, granulates of the present invention are suitable for use “as-is”, or without being combined with or added to other components.

The processes of the present invention generally involve combining an aqueous binder solution containing lactitol and water with particulate lactitol in a high shear granulation apparatus. Various embodiments of the present invention employ particular drying strategies, or protocols to avoid the negative effects of heat on lactitol during the drying process. These and other embodiments of the invention involve pre-treatment (e.g., milling, such as jet milling) of the particulate lactitol under certain conditions prior to introduction into the high shear granulation apparatus. The present invention is also directed to continuous granulation processes, including those where one or more process conditions (e.g., starting material flow rates) are controlled to provide a desired product.

Briefly, therefore, the present invention is directed to processes for preparing a lactitol granulate.

In certain embodiments, the processes comprise introducing a binder solution comprising lactitol and an aqueous solution into a high shear granulation apparatus comprising one or more mixing zones; introducing particulate lactitol into the high shear granulation apparatus; combining the binder solution and particulate lactitol within a mixing zone of the high shear granulation apparatus, thereby forming a particulate lactitol mass; and drying the particulate lactitol mass, thereby forming the lactitol granulate. Drying the particulate lactitol mass comprises: introducing the particulate mass into a first drying zone, the temperature within the first drying zone being below 40° C.; and following removal from the first drying zone introducing the particulate mass into a second drying zone, the temperature within the second drying zone being above 40° C.

In further embodiments, the processes comprise introducing a binder solution comprising crystalline lactitol and an aqueous solution into a high shear granulation apparatus comprising one or more mixing zones; introducing particulate lactitol into the high shear granulation apparatus; and combining the binder solution and particulate lactitol within a mixing zone of the high shear granulation apparatus, thereby forming a particulate lactitol mass. The ratio of the mass flow rate of the particulate lactitol into the high shear granulation apparatus to the mass flow rate of the binder solution into the high shear granulation apparatus is at least 10:1.

In still further embodiments, the processes comprise introducing a binder solution comprising crystalline lactitol and an aqueous solution into a high shear granulation apparatus comprising one or more mixing zones; introducing particulate lactitol into the high shear granulation apparatus; and combining the binder solution and particulate lactitol within a mixing zone of the high shear granulation apparatus, thereby forming a particulate lactitol mass. The mass flow rate of the binder solution into the high shear granulation is controlled to provide a moisture content of the particulate lactitol mass of from about 5 wt % to about 10 wt %.

In even further embodiments, the processes comprise introducing a binder solution comprising crystalline lactitol and an aqueous solution into a high shear granulation apparatus comprising one or more mixing zones; introducing particulate lactitol into the high shear granulation apparatus; combining the binder solution and particulate lactitol within a mixing zone of the high shear granulation apparatus, thereby forming a particulate lactitol mass. Prior to introduction into the high shear granulation apparatus, at least a portion of the particulate lactitol is subjected to a milling operation selected from the group consisting of jet milling or cryogenic milling. In certain embodiments, the particulate lactitol is subjected to jet milling prior to introduction into the high shear granulation apparatus.

The present invention is also directed to particulate lactitol granulate compositions.

In certain embodiments, the compositions comprise a lactitol granulate, wherein the granulate exhibits a tapped density of from about 0.8 g/mL to about 0.9 g/mL.

In other embodiments, the compositions comprise a granulate of lactitol monohydrate, wherein the granulate exhibits a tapped density of at least about 0.75 g/mL; and the composition exhibits a viscosity (flowability) of from about 6000 centipoise (cps) to about 7500 cps.

In still further embodiments, the compositions have a particle size distribution such that: no more than 1 wt % of the composition is retained on a #20 US Standard Sieve; and/or no more than 35 wt % of the composition is retained on a #40 US Standard Sieve; and/or at least 15 wt % of the composition is retained on a #100 US Standard Sieve; and/or no more than 10 wt % of the composition passes through a #200 US Standard Sieve.

The present is also directed to a fast-dissolving particulate lactitol composition (e.g., a lactitol granulate), wherein the composition exhibits a dissolution rate of less than 40 seconds when subjected to laser light scattering analysis.

The present invention is also directed to method for the treatment of constipation, the methods comprising administering to a subject in need thereof a composition comprising the particulate lactitol granulate composition of the present invention.

Other objects and features will be in part apparent and in part pointed out hereinafter.

### BRIEF DESCRIPTION OF THE DRAWINGS

FIG. 1 provides particle size distribution data for a lactitol granulate prepared as described in Example 1a.

FIG. 2 provides a photomicrograph of a lactitol granulate prepared as described in Example 1a.

FIG. 3 provides results of dissolution testing for a lactitol granulate as described in Examine 1a.

FIG. 4 provides particle size distribution data for a lactitol granulate prepared as described in Example 1a.

FIGS. 5 and 6 provide results of dissolution testing as described in Example 1a.

FIGS. 7-12 provide particle size distribution data for milled lactitol powder as described in Example 1a.

FIGS. 13-16 provide photomicrographs of lactitol granulates prepared using jet milling as described in Example 1a.

FIG. 17 provides a photomicrograph of jet milled lactitol prepared as described in Example 1.

FIG. 18 provides a photomicrograph of a lactitol granulation prepared as described in Example 1.

FIG. 19 provides dissolution testing for a lactitol granulate as described in Example 1a.

FIG. 20 provides particle size distribution data for a lactitol granulate as described in Example 1a (Table 8).

FIG. 21 provides results of dissolution testing as described in Example 1a (Table 8).

FIG. 22 provides particle size distribution data for jet milled samples as described in Example 1a (Table 10).

FIGS. 23-24 provide the results of dissolution testing as described in Example 1a.

FIG. 25 provides particle size distribution data for a lactitol monohydrate as described in Example 1b.

FIG. 26 provides a photomicrograph of the lactitol monohydrate sample described in Example 1b.

FIG. 27 provides particle size distribution data for jet milled lactitol as described in Example 1b.

FIG. 28 provides a photomicrograph for jet milled lactitol as described in Example 1b.

FIG. 29 provides particle size distribution data for a lactitol granulate prepared as described in Example 1b (Table 13).

FIG. 30 provides particle size distribution data for a lactitol granulate prepared as described in Example 1a.

FIG. 31 provides a photomicrograph for a lactitol granulation prepared from milled lactitol prepared as described in Example 1b.

FIG. 32 provides lactitol dissolution testing results for lactitol granulations described in Example 1b.

FIG. 33 provides particle size distribution data for a jet milled lactitol as described in Example 2 (Table 14).

FIG. 34 provides particle size distribution data for a lactitol granulate prepared using a two-step drying process as described in Example 2.

FIG. 35 provides dissolution testing results for a lactitol granulate prepared as described in Example 2.

FIG. 36 provides particle size distribution data for a lactitol granulate prepared as described in Example 3.

FIGS. 37-41 provide particle size distribution data for lactitol granulations prepared as described in Example 3.

FIG. 42 provides results of dissolution testing for lactitol granulates prepared as described in Example 3.

FIG. 43 provides in-process moisture testing results as described in Example 4.

FIG. 44 provides tapped density results for lactitol granulates as described in Example 4.

FIG. 45 provides in-process moisture testing results as described in Example 5.

FIGS. 46-47 provide tapped density results as described in Example 6.

FIGS. 48-56 provide results for the twelve month stability test described in Example 7.

### DETAILED DESCRIPTION OF THE INVENTION

The processes of the present invention generally involve preparing granulates of lactitol from particulate lactitol by combining with a binder solution. In accordance with the present invention, the binder solution contains water and dissolved lactitol. Therefore, lactitol functions as the binder for the lactitol granulates. The processes of the present therefore may be referred to as “self-granulation” processes.

Overall, the processes involve combining particulate lactitol with the binder solution in a suitable granulation apparatus. Suitable apparatus include, for example, high shear granulators known in the art.

The binder solution contains water and dissolved lactitol crystals. Generally, lactitol constitutes at least about 20 weight %, at least about 30 weight %, or at least about 40 weight % of the binder solution. Typically, lactitol constitutes from about 30 weight % to about 60 weight %, from about 35 weight % to about 50 weight %, or from about 40 weight % to about 45 weight % of the binder solution.

Preparing the binder solution involves dissolving lactitol crystals in water at a temperature of from about 30° C. to about 40° C., and typically about 35° C. If necessary to provide a well-dispersed binder solution the mixture may be agitated for a certain period of time.

It has been discovered that the particle size distribution of the particulate lactitol may affect control of the granulation process and certain properties of the final granulate (e.g., particle size distribution, dissolution properties, and flowability). Generally, the particulate lactitol has a (d50) particle size distribution (mean particle size by volume distribution) of at least about 25 μm, or at least about 50 μm. Typically, the particulate lactitol has a (d50) particle size distribution such that the average particle size (d50) is from about 25 μm to about 125 μm, or from about 50 μm to about 100 μm.

In order to provide the particulate lactitol of the desired particle size distribution, in certain aspects of the invention the particulate lactitol is subjected to a size reduction unit operation (e.g., milling) prior to its introduction into the high shear granulator. Lactitol is known to have a relatively low glass transition temperature (Tg) (i.e., 32° C. at 4 wt % moisture content). Conventional milling operations may subject the lactitol to temperatures above this limit, which results in annealing of lactitol crystals to form large particles or aggregates that negatively impact the granulation process. In accordance with the present invention it has been discovered that milling techniques that do not result in heat build-up within the milling apparatus and the material being milled are effective in avoiding the effects of heat on lactitol. Such methods include jet milling and cryogenic milling. In certain embodiments, jet milling is the preferred method of milling. In jet milling, air pressure feeds the particles to the mill so that they collide and form smaller particles. The air flow minimizes heat build-up.

Operating conditions for the milling operation may impact whether lactitol having the desired particle size distribution is provided. Typically, the feed injection pressure is from about 25 psi to about 35 psi (e.g., 30 psi). The grinding pressure (which determines how fast the particles are accelerated in the chamber) is typically similar to the feed injection pressure.

Either as-received or after pre-milling, particulate lactitol generally is introduced into the high shear granulation apparatus at a rate of at least about 5 kg/minute, at least about 10 kg/minute, or from about 5 kg/minute to about 15 kg/minute. The binder solution generally is introduced into the high shear granulation apparatus at a rate of at least about 0.4 kg/minute, at least about 0.5 kg/minute, or from about 0.4 kg/minute to about 0.8 kg/minute. The absolute values for these rates will be dependent on the size of the apparatus, desired output, etc.

In the “self-granulation” process of the present invention, typically at least about 90 weight % or at least about 95 weight % of the lactitol is provided by the particulate lactitol, with the binder solution being the source of the remaining lactitol. It has been observed that controlling the relative mass flow rates of the particulate lactitol and binder solution contributes to providing acceptable granulates and acceptable production rates. Typically, the ratio of the mass flow rates of the particulate lactitol to the binder solution introduced into the high shear granulation apparatus is at least about 10:1, at least about 15:1, or at least about 20:1. Generally, this ratio is from about 10:1 to about 30:1, or from about 20:1 to about 30:1 (e.g., about 25:1).

The relative flow rates of the particulate lactitol and binder solution affect the moisture content of the particulate lactitol mass formed within the granulation apparatus. In certain embodiments, the target moisture content of the particulate lactitol mass is less than about 10 weight %, less than about 9 weight %, or less than about 8 weight % (e.g., about 7.5 weight %). The target moisture content of the particulate mass may be from about 5 weight % to about 10 weight %, or from about 6 weight % to about 8 weight %. Therefore, the present invention is directed to embodiments where the flow rates of the particulate lactitol and the binder solution and/or the mass ratio of these two flow rates are controlled in order to provide a particulate lactitol mass having desired moisture content.

The flow of binder solution can also be controlled in order to provide control of the tapped density of the final product. If the tapped density of the lactitol granulates is higher than desired, the flow rate of the binder solution may be increased in order to provide greater wetting of the particulate lactitol and therefore a reduction in the density (including tapped density) of the final granulate product.

Advantageously, the processes of the present invention may be conducted in a continuous manner in which dried lactitol granulate product is recovered while starting materials are introduced into the high shear granulation apparatus and also while particulate lactitol mass is introduced into suitable drying apparatus. Flow rates of the particulate lactitol and/or binder solution are generally controlled in order to provide a desired output of lactitol product and also a lactitol product having the desired properties (e.g., moisture content).

As noted, lactitol exists in the monohydrate, dihydrate, trihydrate, and hexahydrate forms. Lactitol monohydrate is the preferred form of lactitol for use. Lactitol monohydrate is defined by the EU-NP and USP-NF as having moisture content of between 4.5 wt % and 5.5 wt %. As used herein, the term “moisture content” refers to the total moisture content of a composition, including both free and bound water. Moisture contents reported here are determined by the Karl-Fischer (KF) method, but may also be determined by other suitable methods known in the art.

The starting lactitol material in the present processes (of both the binder solution and the particulate lactitol) includes lactitol monohydrate meeting the definitions in the art. The finished product of the present invention also includes lactitol monohydrate meeting the definitions known in the art. However, during processing by combination with the binder solution masses of particulate lactitol are prepared that vary in moisture content as compared to the starting material. Removal of this excess moisture is a key component of the present invention,

For example, combining the binder solution and particulate lactitol in the granulator provides an intermediate particulate mass having higher moisture content than the starting material lactitol monohydrate. Generally, this particulate mass formed within the granulator has a moisture content of from about 6 weight % to about 10 weight % or from about 6 weight % to about 8 weight %.

When subjected to temperatures above its glass transition temperature, particulate masses of lactitol form large masses such as clumps, blocks, etc. Conventional spray drying and other drying apparatus subject the substance being dried to such temperatures. In accordance with the present invention, it has been discovered that if moisture-containing particulate lactitol masses are simply dried by conventional methods that masses including large clumps and/or blocks of material are formed. Masses including these large clumps or blocks do not provide final granulate compositions that exhibit desired properties such as, for example, flowability, dissolution, viscosity, etc. In fact, masses that are not able to be processed by conventional apparatus may be formed such that granulates may not be formed at all.

In accordance with the present invention, it has been discovered that a two-step drying process can be employed, which overcomes these issues. Glass transition temperature is inversely related to moisture content. Therefore, as the moisture content of a particulate mass decreases, its glass transition temperature increases. The two-step drying process of the present invention takes advantage of this phenomenon.

In a first drying step, moisture is removed, which increases the glass temperature of the particulate mass. Unexpectedly, it was discovered that the first drying step can effectively be conducted without elevated temperatures generally believed to be required for drying moist particulate masses. The first drying step of the present invention generally involves subjecting a particulate lactitol mass to temperatures below 40° C. It was further discovered that effective drying in this first step can be provided by agitating the particulate mass while contacted with an oxygen-containing gas (e.g., air) at a temperature below 40° C. This first drying step provides a particulate mass having reduced moisture content, and therefore higher glass transition temperature, as compared to the mass removed from the granulator and introduced into the first drying zone. Generally, the glass transition temperature of the particulate mass exiting the first drying zone will be less than about 60° C., less than about 50° C., or less than about 40° C.

Overall, the first drying step is carried out by subjecting the particulate mass to temperature conditions within the range of from about 0° C. to about 35° C., from about 10° C. to about 35° C., or from about 15° C. to about 30° C. Typically, the temperature of the first drying zone is below the glass transition temperature of lactitol.

Although dependent on the amount of the material introduced into the drying apparatus, generally the particulate mass is within the first drying zone for a first drying period of up to about 20 minutes, up to about 10 minutes, or up to about 5 minutes.

As noted elsewhere herein, glass transition temperature is dependent on moisture content. Therefore, reference to “glass transition temperature” herein refers to the temperature of the particular mass of interest with the understanding that glass transition temperature will between different particulate masses and different samples of particular masses depending on the moisture content.

The second drying step is conducted at higher temperatures to remove remaining moisture from the particulate mass recovered from the granulator and to provide a finished product including lactitol monohydrate. Higher temperatures are permitted in the second drying zone without the negative effect of heat on lactitol due to the decrease in moisture content/increase in glass transition temperature that occurs in the first drying zone. The temperature conditions of the second drying step generally are greater than 40° C., from about 40° C. to about 70° C., from about 40° C. to about 60° C., from about 45° C. to about 60° C., or from about 45° C. to about 55° C. The temperature conditions can be determined and/or monitored at the inlet to the first and second drying zones and/or within the first and second drying zones.

The particulate mass is generally within the second drying zone for a second drying period of up to about 20 minutes, up to about 10 minutes, or up to about 5 minutes.

The two drying zones may be present within the same or separate vessels. In certain embodiments, the first drying zone is contained within or comprised by a separate vessel fitted with suitable apparatus (e.g., for providing air flow, such as paddles) for agitating the particulate mass while subjecting the mass to the temperature conditions of the first drying zone. The second drying zone is then contained within or comprised by a separate vessel where the temperature conditions of the second drying step are imposed. In other embodiments, the two drying zones are contained in a single vessel such as, for example, a horizontal fluid bed dryer. In such instances, a first drying zone will be near the inlet to the fluid bed, while a second drying zone will be near the outlet of the fluid bed, with the bed equipped with suitable mechanism to pass the particulate mass from the first drying zone to the second drying zone to subject the particulate to the different drying conditions.

Generally within the first and second drying zones, the particulate mass is contacted with an oxygen-containing gas (e.g., air) that is either heated or cooled to the desired temperature. Although not critical, purified air heated or cooled to the desired temperate may be introduced into the first drying zone and/or second drying zone.

One-step drying processes under relatively cool temperature conditions similar to the first drying step of the processes of the present invention may under certain conditions (e.g., lengthy drying time) provide a lactitol product having desired moisture content. But such a method does not provide a lactitol granulate exhibiting the other desired properties of the composition of the present invention. In accordance with the present invention it has been discovered that the two-step drying process provides a lactitol granulate having the desired moisture content, but also the other desirable properties of the composition (e.g., quick-dissolving, advantageous flowability). It has also been discovered that these properties are provided without negatively impacting the density (e.g., tapped density) of the granulate composition.

A further advantage of the processes of the present invention is yield of lactitol in the final granulate based on the lactitol introduced into the high shear granulation apparatus (i.e., the total lactitol provided by the particulate lactitol plus the lactitol of the binder solution). Typically, the processes of the present invention provide lactitol yields of at least about 80%, at least about 85%, at least about 90%, or even at least about 95%.

Lactitol Granulates

Lactitol granulates of the present invention are prepared from particulate lactitol combined with a lactitol binder solution. It is currently believed that the granulation process results in a granulate composed of individual lactitol crystals bound together along with water (free and bound) to form the individual particles of the granulate.

As noted, the starting material for the present invention includes lactitol monohydrate, and preferably the lactitol component of the starting material consists essentially of or even consists of lactitol monohydrate. Therefore, in various embodiments the lactitol of granulates of the present invention comprises lactitol monohydrate and likewise preferably consists essentially of or even consists of lactitol monohydrate. Overall, lactitol of the granulates of the present invention is in the crystalline form, and typically a substantial fraction, if not nearly of the lactitol is present in the crystalline form. However, the granulates of the present invention may also contain a fraction of amorphous lactitol monohydrate.

The only components of the granulation process of the present invention are the lactitol and water of the binder solution and the particulate lactitol. It is currently believed that preparing the granulates from these starting materials provides a granulate that contains the individual lactitol crystals in a form that imparts numerous advantageous properties to the granulate. Granulation involves wet massing of the lactitol with shearing forces to form granulates containing individual lactitol crystals, or portions that are joined and bound together along with water to form the granulates of the invention. In short, the granulates of the invention are not simply formed of agglomerated starting material (e.g., lactitol particle or powder), but rather include lactitol crystals bound together by forces imposed during the granulation process that form a composition having various desirable properties. As will be detailed, the granulates of the present invention, which are also referred to herein as “compositions” exhibit one or more desirable properties as compared to powdered lactitol products and other particulate lactitol products.

As noted above, granulates of the present invention are intended to be the composition that is administered (i.e., without adding excipients or other additives). It is to be understood that the granulates are also suitable for use in compositions containing other ingredients.

As noted, powdered lactitol products are known in the art. These compositions, however, suffer one or more disadvantages. For example, these compositions generally exhibit poor viscosity (flowability), poor storage stability (e.g., compaction and clumping), and variations in density over time such that dosing from a container may not remain consistent. Certain aspects of the present invention involve granulate compositions that involve improvements over such compositions.

First, granulates of the present invention exhibit relatively high density as compared to other particulate (e.g., powdered) lactitol formulations. Typically, the present granulates have a bulk density of at least about 0.6 g/mL, or at least about 0.7 g/mL. Overall, the granulates of the composition typically exhibit a bulk density of from about 0.6 g/mL to about 0.8 g/mL, or from about 0.7 g/mL to about 0.8 g/mL.

Tapped density of the granulates of the present invention is typically at least about 0.6 g/mL, at least about 0.75 g/mL, at least about 0.80 g/mL, at least about 0.85 g/mL, or at least about 0.90 g/mL. Overall, the tapped density of the granulates of the present invention generally ranges from about 0.6 g/mL to about 1.0 g/mL, from about 0.7 g/mL to about 0.95 g/mL, or from about 0.8 g/mL to about 0.9 g/mL (e.g., about 0.85 g/mL). Bulk and tapped density as reported herein are determined by USP <616>, but it should be understood that other methods known in the art may be also be utilized to determine the tapped densities of compositions of the present invention.

Granulate density (and in particular tapped density) is an important property as it relates to dosing. Preferred embodiments of the invention are directed to a composition that is packaged and the dosage provided on a volumetric basis (e.g., a scoop). It is currently believed that the tapped densities of the compositions of the present invention provide for consistent dosage amounts by mass for a given dosage volume. In this manner the consumer does not have to weigh the dosage but can simply remove an appropriate volumetric amount from the container (e.g., from a provided scoop or a teaspoon or tablespoon).

The stability of the composition in terms of its density is also an important feature. During storage, the composition does not settle to any significant degree, or at least to any degree that impacts the tapped density. Therefore, over time, the same volumetric dose provides the same dose by mass.

One aspect of the present invention is a fast-dissolving granulate composition that can be administered as a sachet dissolved in water (without heating) by a consumer. Generally, the composition exhibits a rapid rate of dissolution. For example, as determined by MALVERN laser light scattering analysis as detailed herein, the compositions of the present invention are typically dissolved within no more than about 40 seconds, no more than about 30 seconds, or no more than about 20 seconds. In certain embodiments, the compositions of the present invention are referred to as instantly solubilized.

Another advantageous feature of granulates of the present invention is flowability, which for powders is typically reported in terms of viscosity. As used herein, the terms flowability and viscosity are therefore interchangeable. Flowability (viscosity) for the powders of the present invention is determined by the “cup method,” including the method detailed herein (i.e., the HANSON Intrinsic Flowability Test). Other art-accepted methods may be used.

Typically, the viscosity (flowability) of the compositions of the present invention is no more than about 8000 centipoise (cps), or no more than about 7000 cps. Generally, the viscosity (flowability) of the compositions of the present invention is from about 4000 cps to about 8000 cps, from about 5000 cps to about 8000 cps, or from about 6000 cps to about 7500 cps. These represent lower viscosities than those of the particulate lactitol material dissolved in the lactitol solution (e.g., from 9000-10,000 cps) and also the viscosity of the milled lactitol (e.g., greater than 40,000 cps, or even greater than 45,000 cps). As powder viscosity increases, flowability decreases. The increase in flowability because of the reduced viscosity of the compositions of the present invention therefore is an advantage over milled lactitol products. Improved flowability contributes significantly to ease in processing and storage stability.

Particle size distributions of the compositions of the present invention are believed to contribute to one or more advantageous properties of the compositions. Generally speaking, the particle size distributions of the compositions of the present invention indicate a greater proportion of larger particles as compared to other compositions. In various embodiments, the compositions of the present invention exhibit a particle size distribution having a (d10) value of less than about 75 μm (e.g., from about 50 μm to about 75 μm), a (d50) value of less than about 280 μm (e.g., from about 220 μm to about 260 μm), and/or the (d90) particle size is less than about 700 μm (e.g., from about 550 μm to about 700 μm).

Additionally or alternatively, the particle size distribution may be characterized as follows: no more than 1 wt % of the composition is retained on a #20 US Standard Sieve; and/or no more than 35 wt % of the composition (e.g., from about 10 wt % to about 30 wt %) is retained on a #40 US Standard Sieve; and/or at least 15 wt % of the composition (e.g., from about 20 wt % to about 35 wt %, or from about 20 wt % to about 30 wt %) is retained on a #100 US Standard Sieve; and/or no more than 10 wt % of the composition (e.g., less than about 6 wt %, or from about 0 to about 3 wt %) passes through a #200 US Standard Sieve.

Overall, the particle size distributions of granulates of the present invention are believed to indicate the presence of larger and/or more large particles as compared to conventional compositions. This is currently believed to contribute to various advantageous properties of the composition—e.g., relatively high density (bulk and tapped), advantageous viscosity (flowability), and storage stability. Particularly advantageous is the fact that the compositions of the present invention exhibit the dissolution rates detailed herein along with these properties.

Surprisingly it has been discovered that the compositions of the present invention may exhibit higher dissolution rates than conventional, powdered lactitol products. This is currently believed to be due to the nature of granulates provided by the current processes. The high shear granulation process breaks up the individual lactitol crystals, which are then combined with other lactitol crystals from both the particulate lactitol and the binder solution. The individual lactitol crystals are also combined with water from the binder solution. It is currently believed that this method incorporates “semi-bound” water into the granulates. Without being bound by any particular theory, it is currently believed that he granulates containing individual lactitol crystals bound to each other and/or bound and semi-bound water are more easily dissolved through breaking of the bonds between the crystals and water, as compared to other conventional lactitol products that may not include the components arranged in this manner. Additionally or alternatively, it is currently believed that the individual crystal portions of the finished granulates may include slightly annealed portions. Without being bound by any particular theory it is currently believed that these annealed portions of the lactitol crystals may contribute to the advantageous dissolution rates of the compositions of the present invention.

In one aspect the present invention is directed to a lactitol granulate, in particular a lactitol granulate containing lactitol monohydrate. The granulates generally contain individual crystals of lactitol monohydrate bound to/associated with each other to form the granulates containing a plurality of the individual crystals.

In various embodiments, the granulates (i.e., compositions) exhibit high lactitol monohydrate content, in particular, lactitol monohydrate contents of at least about 97 weight %, at least 97.5 weight %, at least about 98 weight %, at least about 98.5 weight %, at least about 99 weight %, at least about 99.5 weight %, at least about 99.6 weight %, at least about 99.7 weight %, at least about 99.8 weight %, or even at least about 99.9 weight %.

In these and other embodiments, the granulates of the present invention also exhibit high purity in terms of crystalline lactitol (as compared to amorphous lactitol). Typically, at least about 95 weight %, at least about 96 weight %, at last about 97 weight %, at least about 98 weight %, at least about 99 weight %, at least about 99.5 weight %, or at least about 99.9 weight %, and preferably all, of the lactitol of the granulates is in crystalline form.

Overall, granulates (i.e., compositions) of the present invention are composed of nearly all and preferably substantially all by crystalline lactitol monohydrate. However, a fraction of amorphous lactitol monohydrate may be present as well.

As noted above, the lactitol granulates of the present invention are suitable for use as a product themselves without formulating with additives, or excipients. In addition to being a stand-alone product, the granulates of the present invention may also be incorporated into compositions along with other additives and excipients.

Methods of Treatment

The present invention is further directed to methods for the treatment of constipation by administering a composition of the present invention to a subject in need thereof. In particular, the present inventive methods are suitable for treatment of chronic idiopathic constipation and opioid induced constipation.

One such method involves treatment using a daily dose of from 5 to 10 g/day of granulates of the present invention. In certain embodiments, the granulates are packaged in a container along with a device for delivering the required dosage amount (by volume) (e.g., a spoon or scoop). Regardless of the precise dosage, advantageously the compositions of the present invention provide a uniform dosage over time whether administered in terms of mass or volume. Typically, over time (e.g., during storage by a consumer of at least 1 month, at least two months, or even at least three months) the compositions of the present invention are suitable for providing a dosage that does not vary by more than 10%, by more than 5%, or by more than 1%.

Having described the invention in detail, it will be apparent that modifications and variations are possible without departing from the scope of the invention defined in the appended claims.

### EXAMPLES

The following non-limiting examples are provided to further illustrate the present invention.

### Example 1: Lab-Scale Granulation—Process Parameters

The following example details a process for preparing a lactitol granulate in accordance with the present invention. The following example demonstrates that controlling the binder feed rate, grinding pressure, and feed injection pressure parameters impacts process performance. Example 1a analyzes raw lactitol (DANISCO), jet milled lactitol, and granulated lactitol. Example 2b analyzes raw lactitol (SHANDONG LUJIAN BIOLOGICAL TECHNOLOGY, China), jet milled lactitol, and granulated lactitol.

Milling the raw lactitol prior to granulation using a STURTEVANT MICRONIZER Jet Mill yielded the best results. Ideal grinder pressure was set at 32 psi and the feed injection pressure was set at 30 psi. These parameters are used for all jet milling processes hereinafter unless otherwise indicated.

Using the jet milled lactitol and minimizing the amount of binder added (slower binder feed rate) was effective in achieving better control of the granulation process and producing an acceptable free flowing lactitol granulation with a finer more consistent particle size with excellent dissolution properties. The dissolution results for the granulations made under these conditions were approximately 8 times faster based on the dissolution test than the dissolution of the raw lactitol itself, as shown below.

Samples were also subjected to flowability testing using a HANSON FLODEX Powder Flowability tester. The basis for the method is the powders ability to fall freely through a hole in a plate. The diameter of the smallest hole through which the powder passes is considered the flowability index. The flowability index (radius of hole size in cm) along with the bulk density of the material tested are then used to determine the viscosity (internal friction coefficient) of the powder. Lower powder viscosity indicates better flowability.

All particle size (PS) analyses were conducted using a ROTAP Particle Size Analyzer and a MALVERN MASTERSIZER 2000, except as otherwise indicated. All particle size data were measured in microns and analyzed via the MALVERN MASTERSIZER 2000.

For Examples 1a and 1b, the desired granulation moisture was 5%, except as otherwise indicated. This percentage equates to a water addition rate of 94.18 g/minute. Based on this water addition rate and the required addition rate for the lactitol in the binder solution, the binder solution as described in Table 1 was prepared.

TABLE 1
Binder solution for 5% moisture

| Ingredient | Quantity (g/min) | % | Batch Amount (grams) |
|---|---|---|---|
| Lactitol (Raw Crystal) | 89.47 | 48.7 | 7,307.5 |
| Potable Water | 94.18 | 51.3 | 7,692.5 |
| Needed Binder Output | 183.65 | 100 | 15,000 |

The binder solution consisting of approximately 49% solids (dissolved lactitol) was prepared using the R&D ADMIX mixer. An endothermic reaction occurred when the Lactitol was dissolved in water. The initial water temperature was ˜30° C. After the lactitol was added and dissolved, the binder solution temperature was 19.5° C. The binder solution was mixed for approximately 30 minutes to ensure all material was dissolved.

Unless otherwise indicated, the following equipment was used in Examples 1a and 1b: R&D TURBULIZER High Shear Granulator, Model TCB; Large ACCURATE Powder Feeder; AEROMATIC Fluid Bed Dryer, Model S-2; ADMIX High Shear Mixer; SWECO Vibratory Sieve, 18″; STURTEVANT MICRONIZER Jet Mill, Model 45DM; Halogen Moisture Balance (to determine moisture levels); MALVERN MASTERSIZER 2000 (particle size analyzer); ROTAP particle size analyzer; and a Tapped Density Tester.

### Example 1a: Lab-Scale Granulation—Lactitol Monohydrate from DANISCO

Characterization of Raw Lactitol Monohydrate

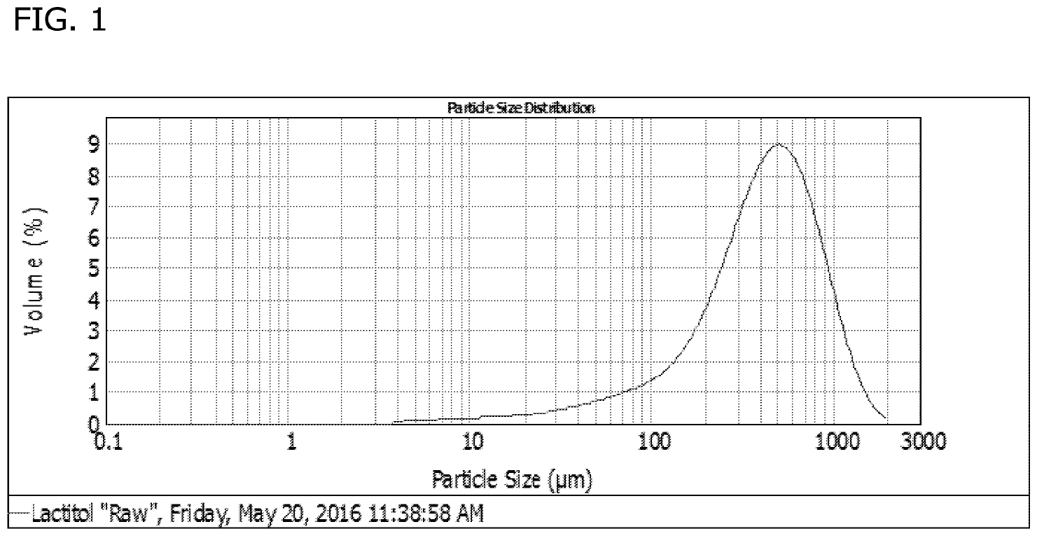

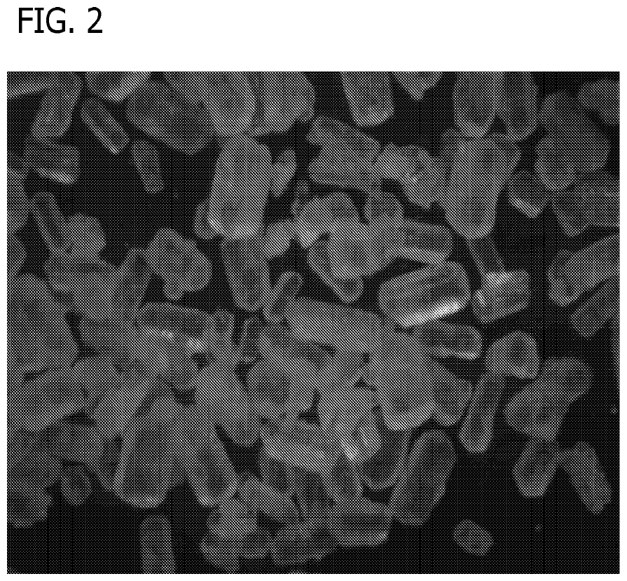

A sample of lactitol monohydrate (DANISCO) was evaluated for particle size moisture (loss on drying (LOD)), bulk density, and tapped density. The results are displayed in Table 2 and FIG. 1. A photo micrograph of the material was also generated (FIG. 2).

TABLE 2
Raw Lactitol Results

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Raw | 0.0 | 10.0 | 57.0 | 26.3 | 4.0 | 0.9 | 1.3 | 0.5 | 112.4 | 426.1 | 922.8 | 0.61 | 0.798 | 0.925 |
| Lactitol | | | | | | | | | | | | | | |
| Preferred | — | >2.0 | 61.0 | 30.0 | 5.0 | — | >2.0 | 0.0 | — | — | — | — | 0.78 | — |

As can be seen in FIG. 2, the lactitol monohydrate had a cylindrical shaped crystal structure. The majority of the material in the sample tested was retained on the 60 mesh screen with none retained on the 12 mesh screen and very few fines. Based on the particle size analysis, the d50 (mean particle size by volume distribution) was 426 microns. The tapped density was 0.93 g/mL.

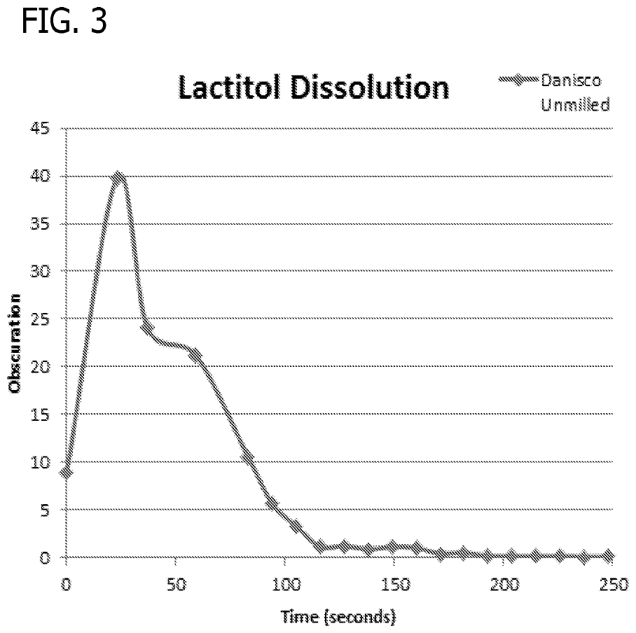

The lactitol monohydrate was also tested for dissolution using the wet sampling attachment for the MALVERN MASTERSIZER 2000. This procedure used 136 mL of water added to the sampler, and then about 2.0 g of lactitol was added with minimal mixing. Snapshots of the particle size distribution were taken over time until the sample appeared fully dissolved as indicated by no change in the obscuration. The results are displayed in FIG. 3. Dispersion of the sample caused a rise in obscuration (blocking of the light by the sample), which fell as the sample dissolves. Later rises in obscuration were likely caused by large aggregated particles breaking into smaller particles, able to block more light before these particles dissolve.

Granulation of Raw Lactitol (Unmilled)

Granulation trials were conducted to determine if raw lactitol (unmilled) could be effectively granulated using the formulation described in Table 3.

TABLE 3
Dry Ingredients for Granulation

| Ingredient | Quantity (g/min) | % | Purpose |
|---|---|---|---|
| Lactitol (Raw Crystal) | 1700 | 95 | Dry Feed |
| Lactitol (in Binder Solution) | 89.47 | 5 | Binder |
| Lactitol Granulation Output | 1789.47 | 100 | |

With the granulator running at full speed, the un-milled lactitol feed rate was initiated at 1700 g/min and the binder feed rate was initiated at ˜184 g/min. The target moisture level at this binder flow rate was 5%. Initially, a very wet granulation was formed. It was not soupy but clumped together very easily. As the process continued the binder flow rate was decreased stepwise from ˜184 to ˜134 g/min before a granulation of acceptable appearance and texture was produced. Approximately 13 kg of wet granulation was produced and collected at this setting. The resultant wet granulation moisture was 1.8%. With the granulator still running, the binder feed rate was increased slightly to ˜150 g/min. An additional ˜13 kilograms of wet granulation were produced at this setting. Again the material displayed acceptable appearance and texture. The resultant wet granulation moisture was 1.9%.

Both wet granulations were dried in multiple sublots in the fluid bed dryer (inlet temperature: ˜58° C.; outlet temperature: ˜45° C.). Significant lumping occurred during the initial drying step that required the drying process to be stopped and the material broken up manually in order to maintain adequate fluidization in the dryer. A lower drying temperature could help minimize the lumping. All 13 kilograms from the first granulation were dried and only 5 kilograms of the second granulation were dried as the lower product container gasket ruptured. The final moisture of the first granulation was 0.82% (Sample 1) and the final moisture of the second granulation was 1.2% (Sample 2).

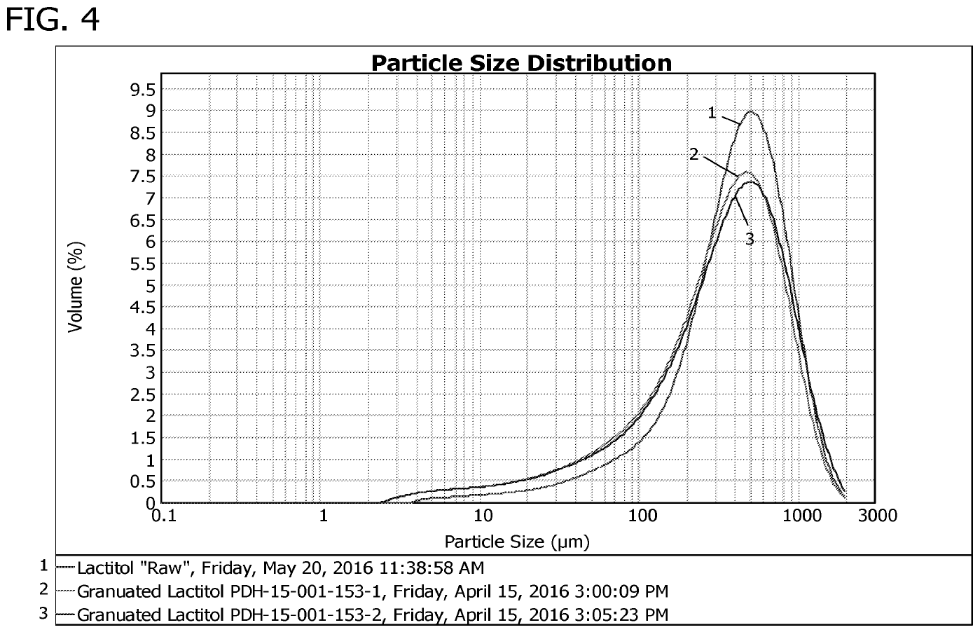

Many lumps were still present after the drying step described above. To eliminate them, each granulation was passed through the R&D U5 COMIL® at slow speed (1700 rpm) with a very large, 0.250″ cheese grater screen. Samples of each granulation along with a sample of the raw lactitol were tested particle size, moisture, bulk and tapped density. The results are displayed in Table 4 and FIG. 4.

TABLE 4
Raw Lactitol and Granulations 1 and 2 results

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Raw | 0.0 | 10.0 | 57.0 | 26.3 | 4.0 | 0.9 | 1.3 | 0.5 | 112.4 | 426.1 | 922.8 | 0.61 | 0.798 | 0.925 |
| Lactitol | | | | | | | | | | | | | | |
| 1 | 4.5 | 8.9 | 42.9 | 30.9 | 7.2 | 2.2 | 3.1 | 0.3 | 58.6 | 352.2 | 859.7 | 0.82 | 0.757 | 0.863 |
| 2 | 5.2 | 13.0 | 41.7 | 27.5 | 6.7 | 2.1 | 3.1 | 0.7 | 60.7 | 371.4 | 933.4 | 1.19 | 0.743 | 0.871 |

As expected, the particle size distributions of the two granulations were coarser than the raw lactitol especially on the 12, 80, 100, and 200 screens. The coarser particle size associated with the two granulations is also depicted in the particle size plots shown in FIG. 4. In addition, there was a slight decrease in product density after the granulation process.

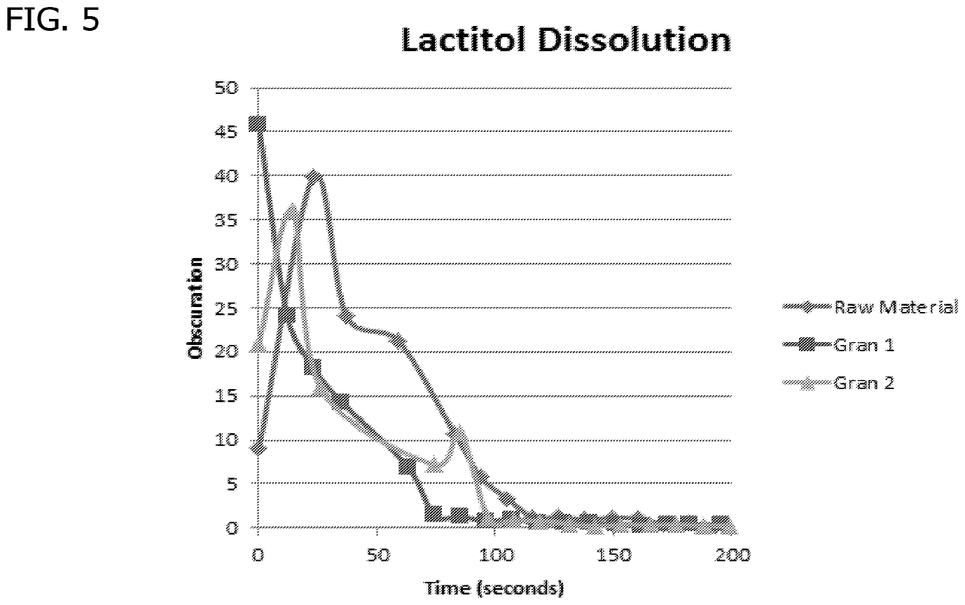

A study was initiated to evaluate the comparative dissolution rates of granulated lactitol using the MALVERN MASTERSIZER 2000. Both samples were evaluated using the same method previously developed for the lactitol raw material (see above). The instrument was set up to perform readings every 10-15 seconds. Although the final laser obscuration did not reach the exact same end point observed for the API, the samples did reach a steady state and initial dissolution rates were able to be compared. Dissolution profiles are depicted in FIG. 5.

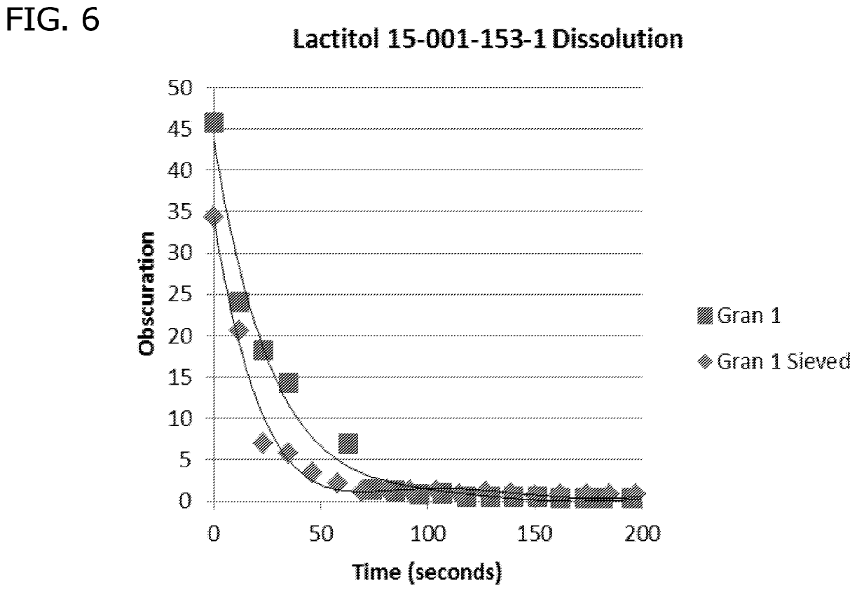

The suspected reason for the delay in completion of the dissolution was some hard, aggregated particles that would fall apart causing a sudden rise in obscuration. To test this, a portion of the sample was sieved through a 20 mesh screen and retested. The results are shown in FIG. 6.

Although the initial dissolution rate did improve, there was still a delay in completion. Comparison of the overlays for the granulated and raw materials shows there is an extra mode in the particle size in the granulated material, suggesting a slightly different mechanism for dissolution.

Milling of Raw Lactitol Prior to Granulation—QUADRO U5 COMIL®

Based on the granulation results above, it was determined that the use of unmilled lactitol creates variability in the granulation process. With the unmilled raw lactitol, controlling the granulation process was difficult and a lot of oversize material was produced resulting in a coarser than desired particle size distribution. Consequently, an evaluation was conducted to determine whether the R&D U5 COMIL® could be used to mill the lactitol to a finer particle size that is more suitable for a granulation process (d50˜100 microns). The particle size of the commercial raw lactitol (d50˜426 microns) is very large resulting in a reduced surface area needed for effective binder absorption during granulation. Larger particles easily over granulate with minimal amount of water (binder) added.

For this study, one scoop of the raw lactitol was passed through the Model U5 QUADRO COMIL® using various screen configurations and mill speeds. Particle size testing was conducted on each sample. The best results were achieved using a 0.032″ round hole screen with a square bar impeller at a speed of 4200 rpm. The d50 at these conditions was approximately 115 microns. An attempt was made to mill a larger quantity of lactitol using these same conditions. However, after approximately 2 minutes of milling, the mill overloaded causing it to stop. Upon inspection, the lactitol melted under the impeller bar and extruded through the screen. The heat generated during the milling process softens the lactitol due to its low glass transition temperature (˜32° C. at 4% water). Some additional trials were conducted at lower mill speeds with a 0.032″ round hole screen with a round bar impeller. Mill speeds of 1500 and 1800 rpm were also tested. As expected, the particle size achieved at the lower mill speeds is larger than desired (d50 at 1800 rpm=196 microns; d50 at 1500 rpm=235 microns). Also after approximately 4 minutes at 1800 rpm and after approximately 10 minutes at 1500 rpm the lactitol began to stick on the impeller and screen. Based on these results, the COMIL® was shown inadequate for the lactitol milling step. Another lower energy, less aggressive milling method needs to be evaluated for this application (i.e., jet milling, cryogenic milling etc.).

Milling of Raw Lactitol Prior to Granulation—STURTEVANT MICRONIZER Jet Mill, Model 45DM

A study was initiated to evaluate the use of a STURTEVANT MICRONIZER Jet Mill, Model 45DM to mill the raw lactitol into a fine powder suitable for granulations. The jet mill uses air pressure to feed the product to the mill and to accelerate the particles in the milling chamber so they collide into one another shattering them into smaller pieces. The air flow through the mill helps to mitigate heat build-up. The jet mill has two controls for adjusting the particle size during milling. The feed injection pressure determines how much and how fast the material is introduced into the grinding chamber and the grinding pressure determines how fast the particles are accelerated in the grinding chamber where particle collisions occur.

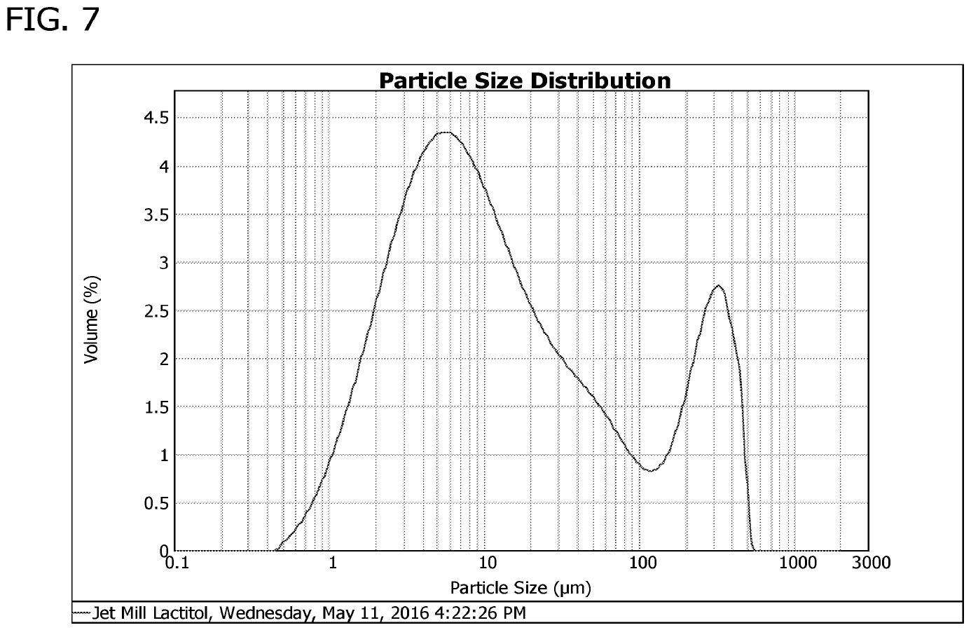

Through initial range finding studies it was determined that higher milling pressures (>50 psi) produced extremely fine particles as seen in FIG. 7. The d10, d50, and d90 particle size for the sample displayed are 2.1, 10.0, and 254.4 microns respectively.

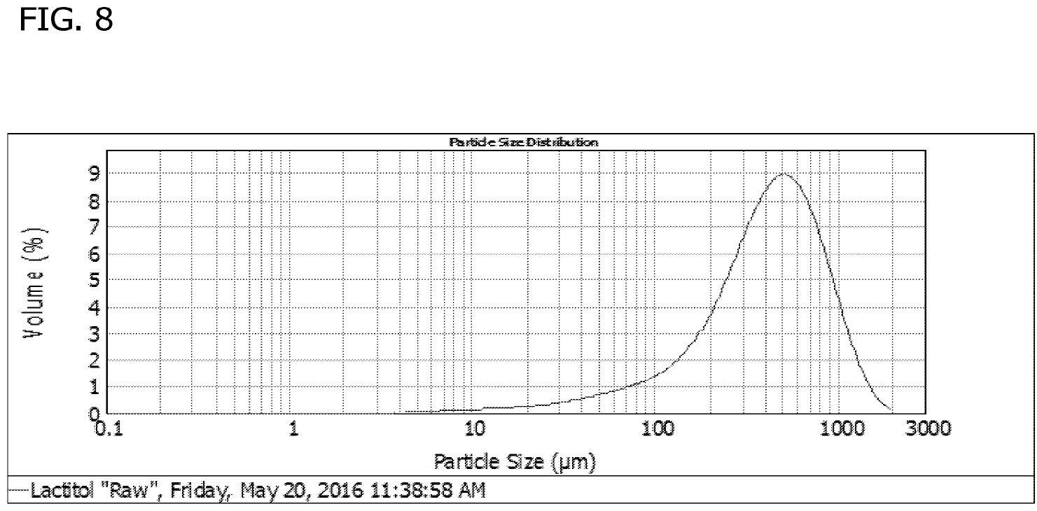

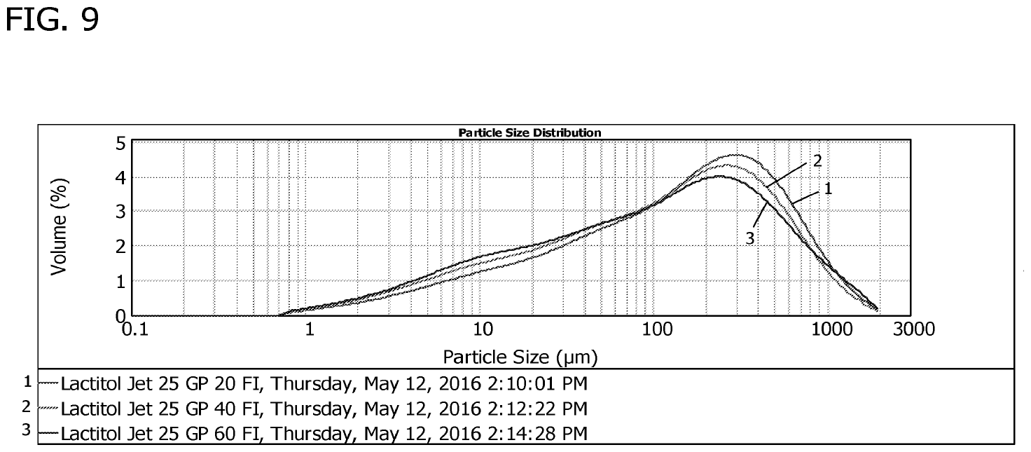

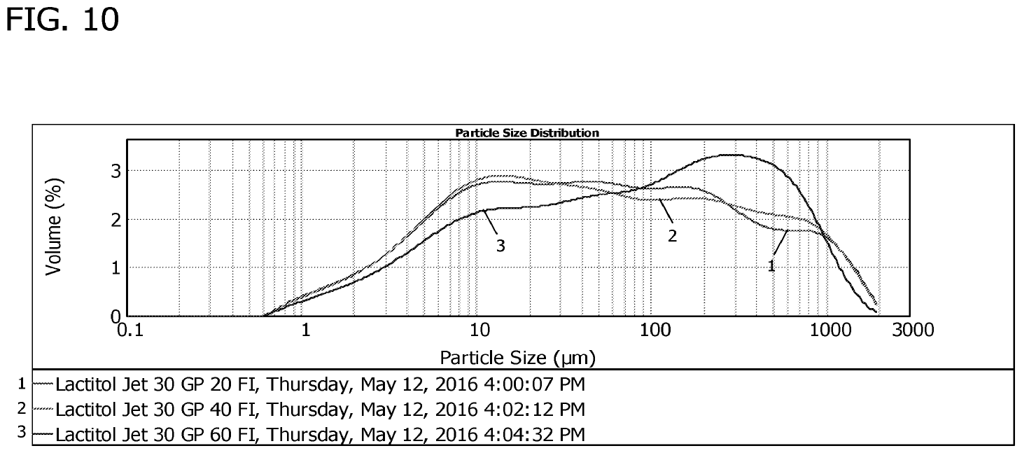

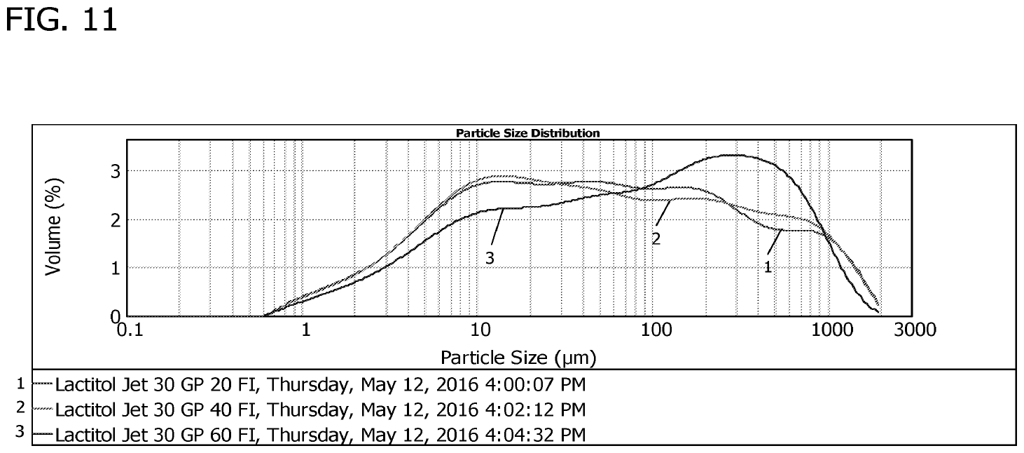

In light of the above studies, lower grinding pressures were selected to optimize milling conditions for the jet mill. The following milling conditions were used to mill Lactitol samples for particle size analysis to determine the best milling conditions to achieve a d50 particle size of approximately 50 to 100 microns. Both the milling conditions and the particle size results are displayed below in Table 5 and FIGS. 8-11.

TABLE 5
Variation of Grinding Pressure and Feed Injection Pressure

| Grinding | Feed Injection | Particle Size (μ) | | | |
|---|---|---|---|---|---|
| Pressure (PSI) | Pressure (PSI) | d10 | d50 | d90 | Span |
| Raw Lactitol (unmilled) | | 112.4 | 426.1 | 922.8 | 1.9 |
| | 20 | 10.8 | 150.7 | 646.1 | 4.2 |
| 25 | 40 | 8.5 | 122.9 | 585.1 | 4.6 |
| | 60 | 5.5 | 84.3 | 619.5 | 7.2 |
| | 20 | 4.5 | 47.8 | 615.2 | 12.7 |
| 30 | 40 | 4.5 | 46.3 | 640.1 | 13.7 |
| | 60 | 4.5 | 45.6 | 513.0 | 11.1 |
| | 20 | 4.1 | 36.0 | 635.0 | 17.5 |
| 35 | 40 | 5.8 | 83.4 | 548.6 | 6.5 |
| | 60 | 7.6 | 110.9 | 611.9 | 5.4 |

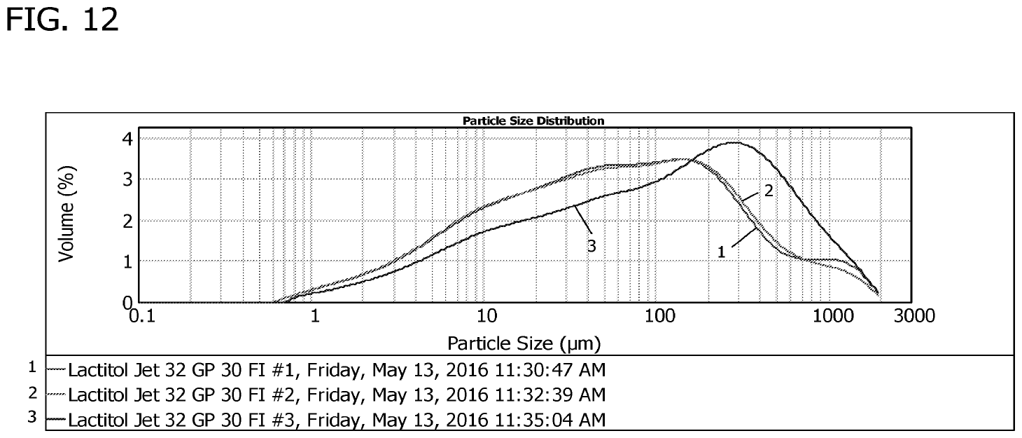

Based on the above results, it was decided that a grinding pressure between 30 and 35 psi and a feed injection pressure of approximately 30 psi would produce a product with a d50 closest to 50 microns. To test this, approximately 12 kg of raw Lactitol was charged to the Accurate powder feeder. The feeder was positioned over the jet mill hopper and adjusted to maintain a steady flow. The grinder pressure was set to 32 psi and the feed injection pressure was set a 30 psi. The entire 12 kilograms was milled in 9 minutes 33 seconds or 75.4 Kg/Hr. Three samples were collected from the drum: samples 1b and 2b just under the surface of the powder (different sides) and sample 3b was taken near the bottom of the container and tested for particle size. As can be seen in the Table 6 and FIG. 12, a d50 particle size of approximately 58 microns was achieved. Sample #3b appears to be coarser than the other samples and may have agglomerated since the sample was taken from the bottom of the drum.

TABLE 6
Particle Size Measurements from Different Areas of the Drum
(GP: 32 PSI, FPI: 30 psi)

| | Particle Size (μ) | | | |
|---|---|---|---|---|
| Sample | d10 | d50 | d90 | Span |
| 1b | 5.6 | 56.7 | 420.9 | 7.3 |
| 2b | 5.5 | 56.5 | 401.2 | 7.0 |
| 3b | 7.6 | 113.1 | 656.4 | 5.7 |

Overall, the jet milling process was effective in reducing the particle size of the raw lactitol without the inherent melting issues experienced with the QUADRO COMIL® due to heat build-up during the milling process. Surprisingly, the raw lactitol could be milled in the R&D jet mill at a rate of approximately 75 Kg/min. Flowability was subsequently tested. For the raw lactitol the viscosity was approximately 9,776 cps and for the jet milled lactitol the viscosity was approximately 4.8 times higher at 46,905 cps.

To ensure the ability to scale up the process to higher milling rates a STURTEVANT FMC 350 air driven classifying impact mill was evaluated. The FMC 350 was found to produce appropriate particle size distribution of the raw lactitol at rates up to 1500 Kg/hr.

Granulation of Jet Milled Lactitol

A granulation trial was conducted with jet milled lactitol. An additional quantity of jet milled lactitol was prepared as described above and combined with the 12 kg to ensure enough material was available for the granulation process. The formulation described in Table 1 was also used for this trial. The binder solution was prepared using the mixer. Once completed the binder was charged to the granulator binder feed tank and continuously mixed at low speed.

With the granulator running at full speed, the jet milled lactitol feed rate was initiated at 1700 g/min and the binder feed rate was initiated at ˜185 g/min. The target moisture level at this binder flow rate is 5%. At these initial conditions, a granulation of acceptable texture and feel was produced. No further adjustments to the operating conditions were needed during the run. Approximately 33 kg of wet granulation was produced and collected at these feed rate settings. The resultant wet granulation moisture was ˜2%. This was lower than expected based on the binder flow rate used.

The wet granulation was dried in multiple sublots (6) in the fluid bed dryer (inlet temperature: 55-59° C.; outlet temperature: 45-50° C.). After drying all of the material was collected in a poly lined drum. A total of 30.2 kg of “as dried” product was recovered. The dried material was then sieved through a 24 TBC screen. A total of 19.6 kg passed though the screen and 10.4 kg was collected as oversize (˜34% of total oversize produced). The “as dried” moisture of the sieved material was approximately 1.4%.

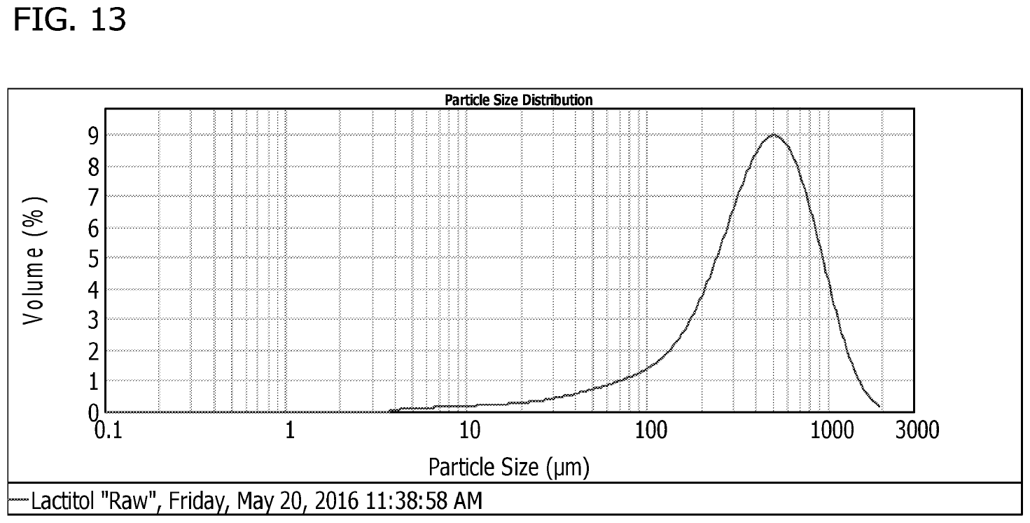

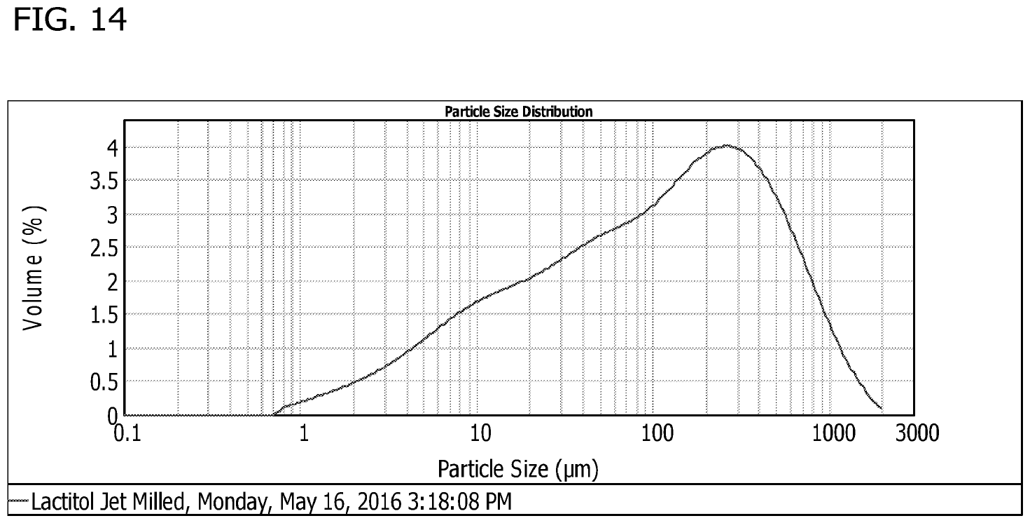

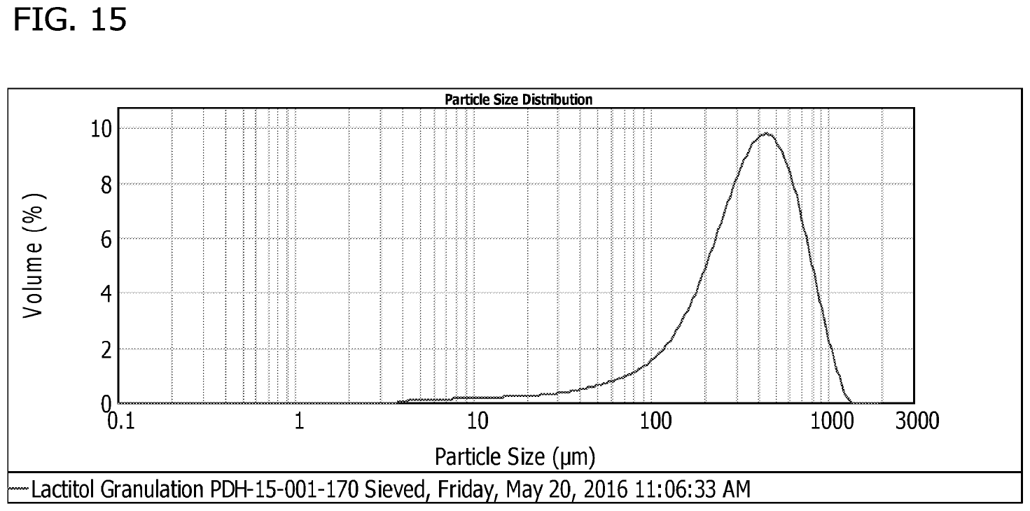

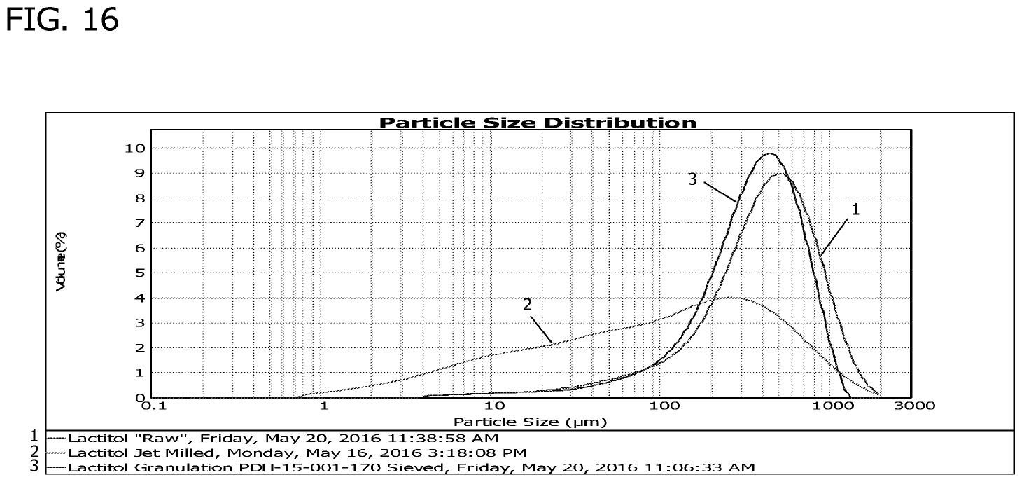

A sample of the granulation (sample 7) along with a sample of the jet milled lactitol (sample 6) were tested for particle size, moisture, bulk and tapped density. The results are displayed in the Table 7 and FIGS. 13-16. Photos of the raw lactitol, jet milled lactitol, and lactitol granulation are also provided for comparison purposes in FIGS. 1, 17, and 18.

TABLE 7
Comparison of Raw Lactitol, Jet Milled Lactitol, and Granulated Lactitol

| | | | | | | | | | | | | | | | Flow |
|---|---|---|---|---|---|---|---|---|---|---|---|---|---|---|---|
| | | | | | | | | | | | | Moisture | Bulk | Tapped | Viscosity |
| | Particle Size Distribution (% Retained) | | | | | | | | PS (μ) | | | NMT | Density | Density | CPS |
| Sample | 12 | 20 | 40 | 60 | 80 | 100 | 200 | Pan | d10 | d50 | d90 | 3.0% | g/mL | g/mL | (×1000) |
| Raw | 0.0 | 10.0 | 57.0 | 26.3 | 4.0 | 0.9 | 1.3 | 0.5 | 112.4 | 426.1 | 922.8 | 0.61 | 0.798 | 0.925 | ~10 |
| Lactitol | | | | | | | | | | | | | | | |
| 6 | 0.0 | 2.6 | 34.9 | 23.2 | 10.7 | 3.7 | 15.5 | 9.3 | 7.8 | 110.4 | 590.9 | — | 0.7.36 | 0.923 | ~50 |
| 7 | 0.0 | 0.1 | 39.6 | 421 | 11.1 | 2.9 | 3.6 | 0.5 | 117.9 | 370.2 | 740.9 | 137 | 0.743 | 0.824 | ~12 |

As shown above in FIGS. 13-16, it was demonstrated that an acceptable free-flowing granulation with a finer more consistent particle size was produced from the jet milled lactitol. Better control of the granulation process was achieved and significantly less oversize (˜24 mesh) was produced compared to the granulation produced with the raw, unprocessed lactitol discussed above. This resulted in little or no material retained on the 12 and 20 mesh screen and in increased amount retained on the 60 mesh screen. Granulation bulk and tapped densities were approximately the same.

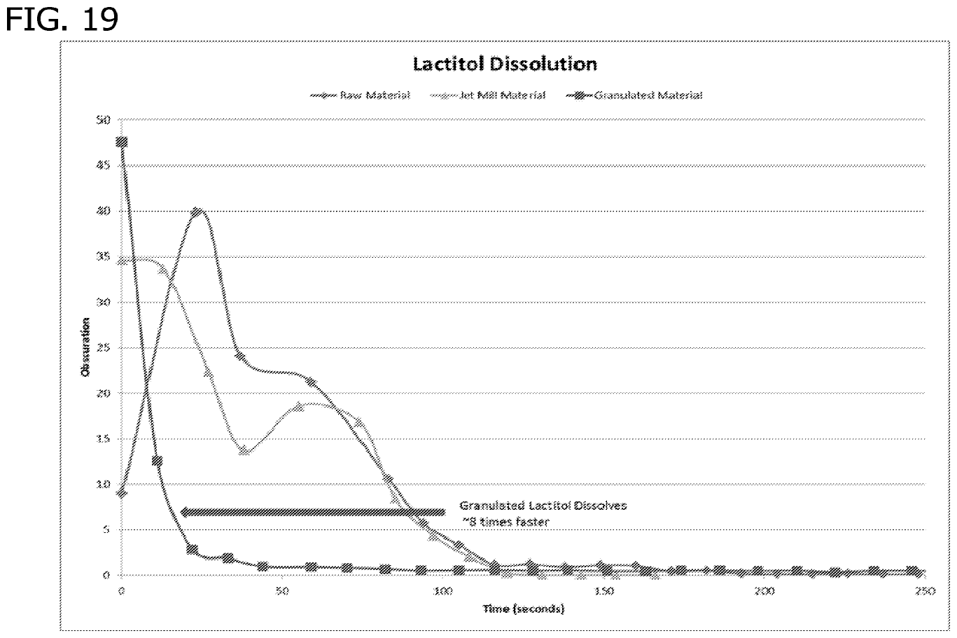

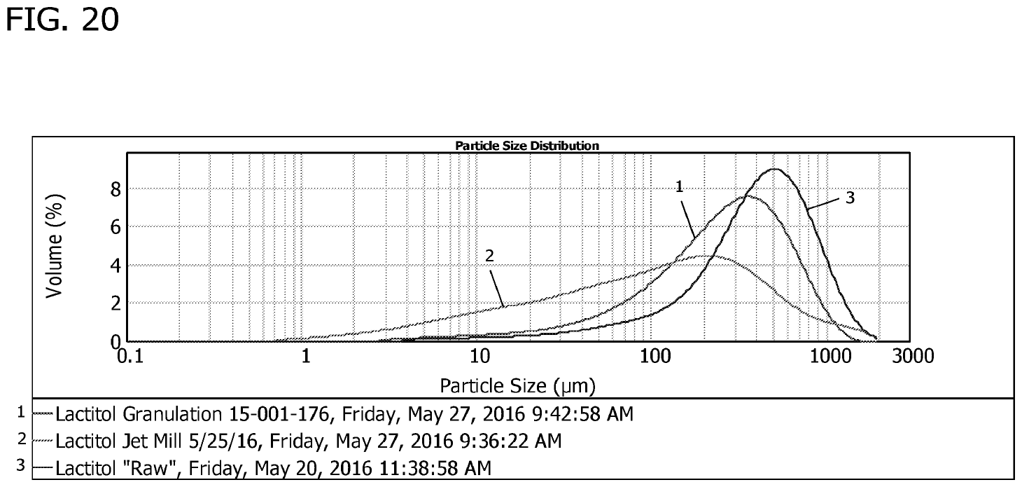

The raw lactitol, the jet milled lactitol, and the lactitol granulation produced above were also tested for dissolution test described above. The results are displayed in FIG. 19. As seen, the dissolution of the granulated lactitol is approximately 8 times faster than the both the raw lactitol and the jet milled lactitol.

A second granulation trial was performed to evaluate whether a lower granulation moisture (lower binder feed rate) will result in a reduced amount of oversize (Sample 8). For this run, approximately 60 kg of the raw lactitol was milled in the jet mill. The binder solution was prepared as described above.

With the mixer running at full speed, the jet milled lactitol feed rate was initiated at 1700 g/min and the binder feed rate was initiated at ˜125 g/min (˜20.5 Hz). At approximately 125 g/min, the expected moisture level would be approximately 3.4%. However, at this initial binder flow rate, the granulation exiting the mixer felt too dry and was not as granular as desired. To improve the granulation quality, the binder flow rate was systematically increased until a granulation of acceptable texture and feel was produced. The final setting was 37 Hz or approximately 140 g/min. At this binder flow rate the expected granulation moisture level would be approximately 3.8%. No additional adjustments were made to the operating conditions over the remainder of the run. The final wet granulation moisture was approximately 2%. In order to improve the accuracy of the LOD moisture results the moisture balance test conditions were altered. The test temperature was increased to 105° C. from 90° C. and the test was run for 10 minutes instead of 5 minutes.

The same drying procedure was used as discussed above. A total of 14.2 Kg passed though the screen and 3.7 kg was collected as oversize (˜20% oversize). This is a reduction of approximately 15% in oversize compared to the previous granulation which used a higher binder flow rate and moisture target of 5%. The “as dried” moisture of the sieved material was approximately 1.4%.

A sample of the granulation along with a sample of the jet milled lactitol were tested for particle size, moisture, bulk and tapped density. The results are displayed in Table 8 and FIG. 20.

TABLE 8
Comparison of Raw Lactitol, Jet Milled Lactitol, and Lower Moisture Granulated Lactitol

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Raw | 0.0 | 10.0 | 57.0 | 26.3 | 4.0 | 0.9 | 1.3 | 0.5 | 112.4 | 426.1 | 922.8 | 0.61 | 0.798 | 0.925 |
| Lactitol | | | | | | | | | | | | | | |
| 6 | 0.0 | 1.5 | 27.5 | 25.3 | 13.8 | 7.5 | 17.1 | 7.4 | 6.6 | 109.3 | 513.4 | — | 0.776 | 0.955 |
| 8 | 0.0 | 0.0 | 25.9 | 33.9 | 15.1 | 6.8 | 15.1 | 3.2 | 60.9 | 271.5 | 665.4 | 1.38 | 0.747 | 0.829 |

As expected, particle size of the material produced using the slower binder feed rate is finer than the granulation produced at the higher binder feed rate. The amount of material retained on the 40 and 60 mesh screens was reduced slightly with the amount on the 80, 100 and 200 mesh increasing by a small to a moderate amount.

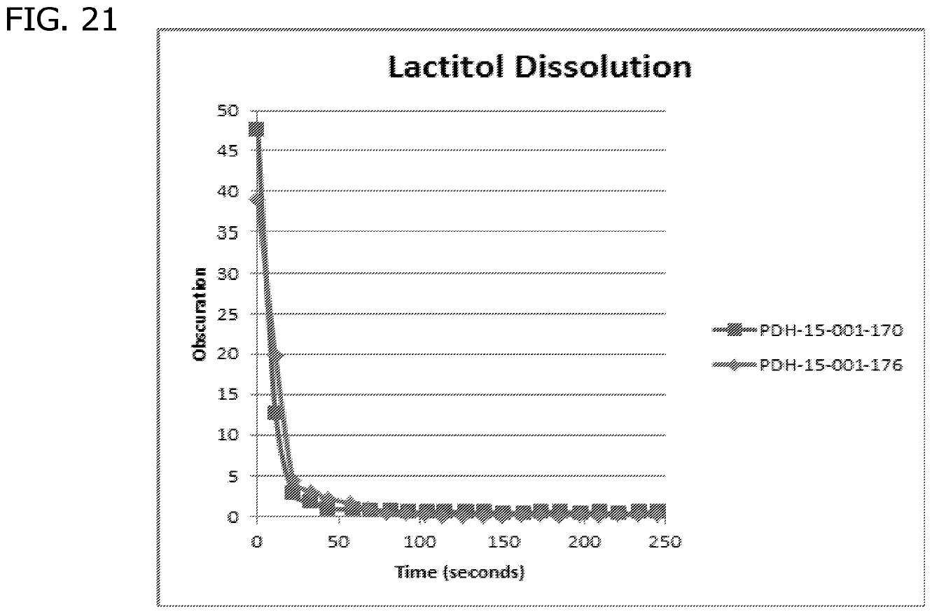

The lactitol granulation produced above was tested for dissolution using the same procedure described above and the results were compared to the results previously generated on lactitol granulation (produced with higher granulation moisture). The results are displayed in FIG. 21. While the particle size of granulation was slightly finer than the previous granulation with higher granulation moisture, the dissolution rates of the two granulations were shown to be about the same (within experimental error).

Jet Milling of Granulation Oversize

A secondary study was conducted with granulation sample 8 to determine the feasibility of using the jet mill to process the oversize produced from the lactitol granulation process. To do this, the plus 24 TBC oversize from the granulation (˜3.7 Kg) was hand sieved through a 12 mesh screen to remove the excessively large oversize material that would not pass through the hopper-inlet tube of the jet mill, using the parameters of Table 9.

TABLE 9
Oversize Jet Milling Parameters

| Sample | Grinding Pressure (psi) | Feed Injection Pressure (psi) |
|---|---|---|
| 10 | 32 | 30 |
| 11 | 40 | 30 |

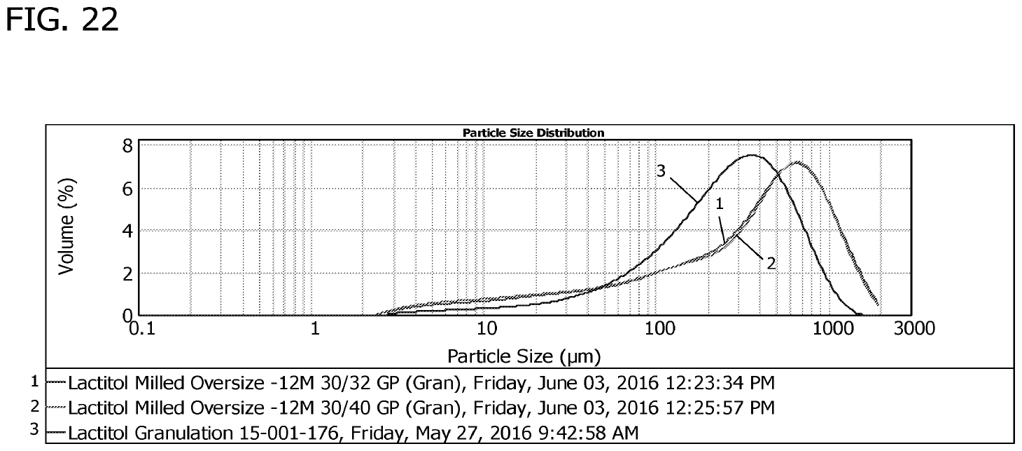

These samples along with a sample of the minus 12 mesh oversize (unmilled) (Sample 9) were tested for particle size and density. See Table 10 below. The two milled samples were also tested for particle size and the results are displayed in FIG. 22.

TABLE 10
Comparison of Granulated Lactitol, Unmilled Oversize Lactitol, and Oversize Jet Milled Lactitol

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| 8 | 0.0 | 0.0 | 25.9 | 33.9 | 15.1 | 6.8 | 15.1 | 3.2 | 60.9 | 271.5 | 665.4 | 1.38 | 0.747 | 0.829 |
| 9 | 0.2 | 33.5 | 58.5 | 5.1 | 1.1 | 0.4 | 0.8 | 0.4 | * | * | * | — | 0.678 | 0.797 |
| 10 | 0.0 | 6.6 | 40.2 | 16.3 | 8.5 | 3.8 | 16.2 | 8.5 | 31.9 | 413.1 | 1069.4 | — | 0.760 | 0.988 |
| 11 | 0.0 | 6.4 | 39.7 | 15.7 | 8.9 | 4.0 | 18.8 | 6.6 | 26.4 | 406.1 | 1050.8 | — | 0.753 | 0.962 |

* particle size of oversize sample was too large to be tested

As can be seen above, the particle size of Samples 10 and 11 were approximately the same. While a significant reduction in the particle size was realized by jet milling the −12 mesh, +24 TBC oversize fraction (samples 10 and 11), the particle size was still coarser than the particle size of the −24 TBC finished product (sample 8). The amount on the 20 and 40 mesh screens was higher with slightly less material on the 60 and 80 mesh screens. As expected, the amount in the pan (fines) was higher for the milled oversize (samples 10 and 11) than displayed by the un-milled finished product (sample 9).

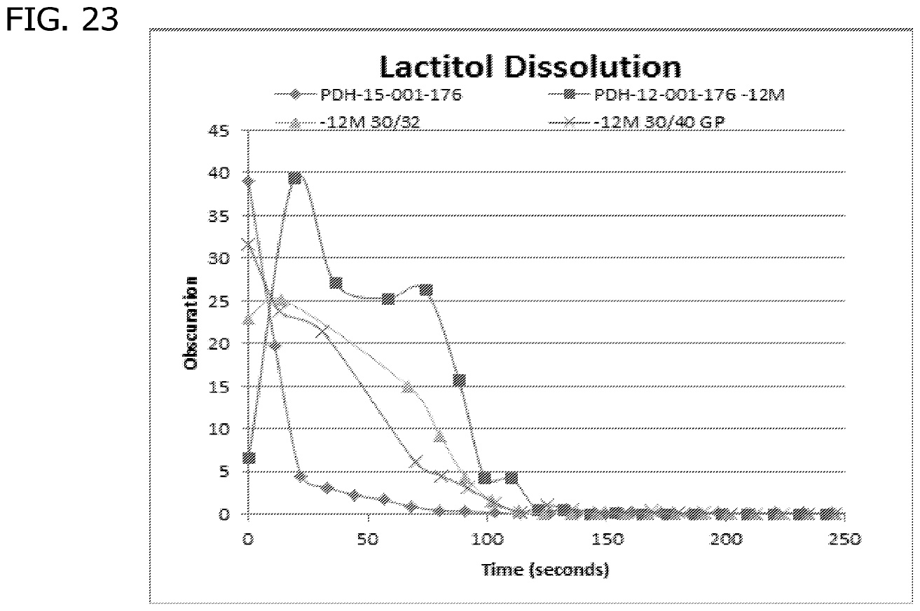

Despite the particle size of the jet milled oversized material being coarser than the finished granulation, it is possible that this material could be recycled during the sieving process if a jet mill was incorporated downstream into the production process. Since the jet milled oversize represents ˜20% of the granulation of the lactitol, the impact of the coarser particle size of the jet milled material on the particle size of the finished granulation is expected to be minimal. To determine what impact the jet milling has on the dissolution rate of the oversize, samples 10 and 11 were tested for dissolution rate. Results were compared between the dissolution rate of Sample 8 and 9. The dissolution profiles are displayed in FIG. 23.

As expected, the initial dissolution rate of the oversized material is considerably slower than the initial dissolution rate of sample 8 due to its larger particle size. Jet milling of the oversized material resulted in an improved dissolution rate; however, the dissolution rate still did not match the finished product. It was also shown that sample 11 dissolved faster initially than sample 10; however; the final dissolution rate of both jet milled oversized material were about the same.

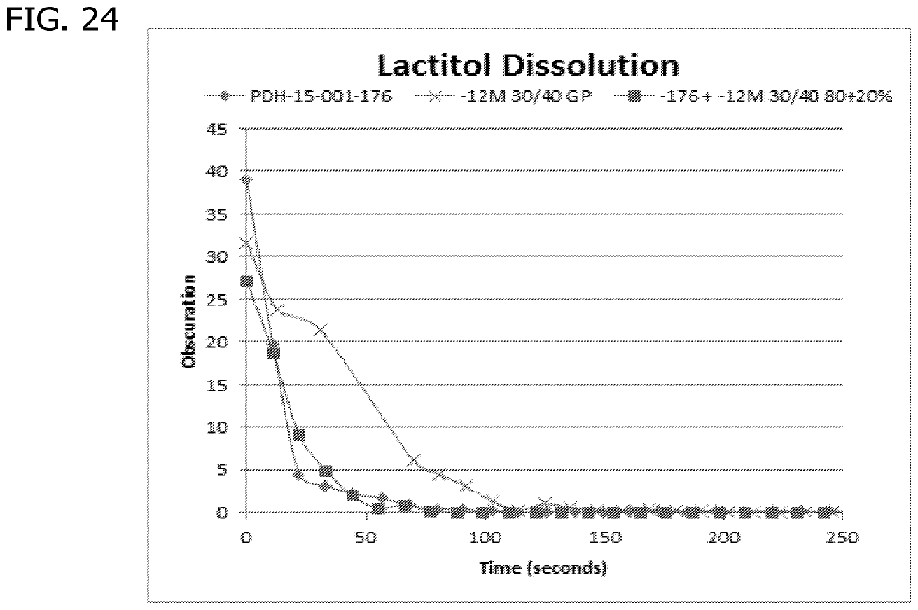

In order to see how recycling milled oversized granulation (approximately 20% of total granulation produced) would affect the dissolution of the final granulation, sample 11 was mixed with lactitol granulation at a ratio of 20:80 (sample 12) to simulated process output with the jet mill integrated downstream in the process. A sample of this mixture was tested for dissolution, along with sample 8 and sample 11. The dissolution profiles are displayed in FIG. 24.

As shown, the dissolution of sample 12 closely matches the dissolution profile of sample 8. Based on these results, it appears feasible to recycle jet milled minus 12 mesh oversize back into the stream of product entering the vibratory sifter during processing without having a significant impact on the final product dissolution properties.

The above experimental investigation demonstrates the general technical feasibility of producing a lactitol granulation using high shear granulation technology. It was demonstrated that raw lactitol could be granulated using an approximately 49% lactitol binder solution. However, granulation process control was difficult due to the large particle size of the raw lactitol and a significant amount of oversize was generated resulting in numerous hard, aggregated particles in the finished product before and even after sizing. Through additional trials, it was shown that a more controlled granulation process and significantly less oversize could be achieved by milling the lactitol prior to use. The lactitol specifically had to be milled using a jet mill as other more aggressive milling techniques such as a COMIL® generate a significant amount of heat during operation causing the lactitol to soften and extrude through the screen.

Through a secondary study it was demonstrated that the ˜20% oversize (˜12 mesh, +24 mesh) generated during the lactitol granulation process can be jet milled and added back to process through the vibratory sifter with only minimal impact on the finished product particle size, density, and dissolution rate.

### Example 1b: Lab-Scale Granulation—Lactitol Monohydrate (Shandong Lujian Biological Technology, China)

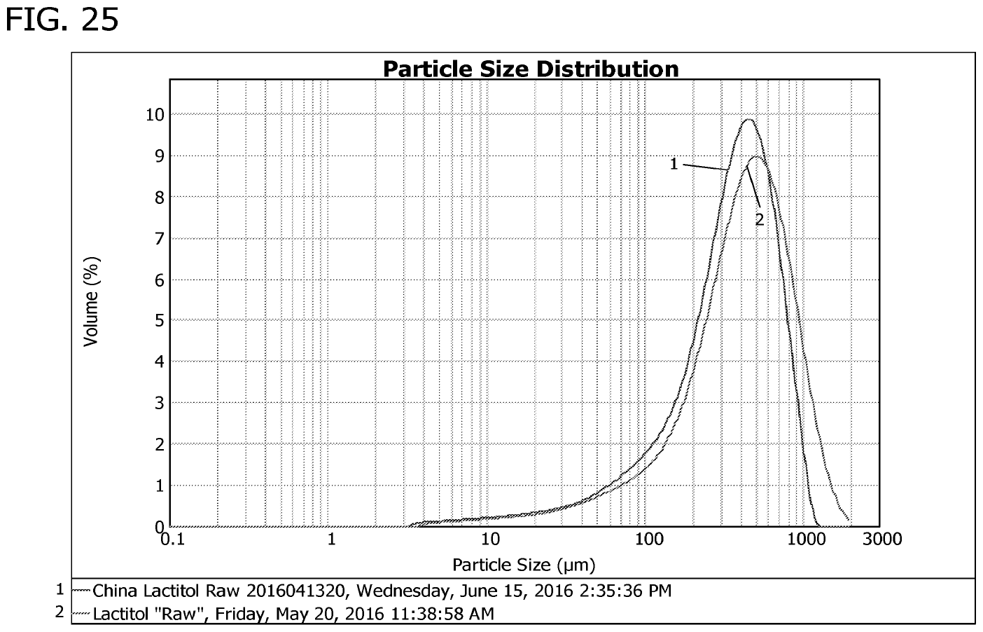

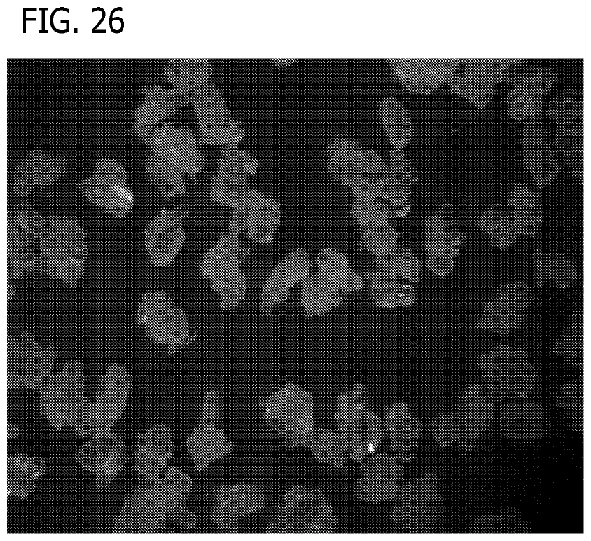

An additional, lower cost source of lactitol monohydrate was evaluated for possible use in the lactitol granulation. Approximately 175 kilograms (25 kg/bag) of lactitol monohydrate was received from the customer through Lincoln Fine Ingredients from SHANDONG LUJIAN BIOLOGICAL TECHNOLOGY CO., LTD., China (hereinafter Lactitol B). A sample of this material was evaluated for particle size, moisture (LOD), bulk density, and tapped density and compared to the DANISCO material previously characterized in Example 1a (hereinafter Lactitol A), and using the same procedures outlined in Example 1a. The results are displayed in Table 11 and FIG. 25. A photo micrograph of the material was also generated (See FIG. 26).

TABLE 11
Comparison of Raw Lactitol from two sources

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Raw Lactitol | 0.0 | 10.0 | 57.0 | 26.3 | 4.0 | 0.9 | 1.3 | 0.5 | 112.4 | 426.1 | 922.8 | 0.61 | 0.798 | 0.925 |
| A | | | | | | | | | | | | | | |
| Raw Lactitol | 0.0 | 0.3 | 64.4 | 29.9 | 3.6 | 0.7 | 0.8 | 0.3 | 94.2 | 365.3 | 720.3 | 1.28 | 0.677 | 0.754 |
| B | | | | | | | | | | | | | | |

As can be seen above, the raw Lactitol B has a finer particle size and a lower density than the A material. The B material is shown in FIG. 26; the crystals are smaller and irregularly shaped with many smaller crystals attached to larger crystals (see FIG. 1 for comparison). This observation indicates that the Lactitol B crystals are likely produced using a different process (possibly a much faster recrystallization step) than the A Lactitol.

Jet Milling of the Lactitol Monohydrate B

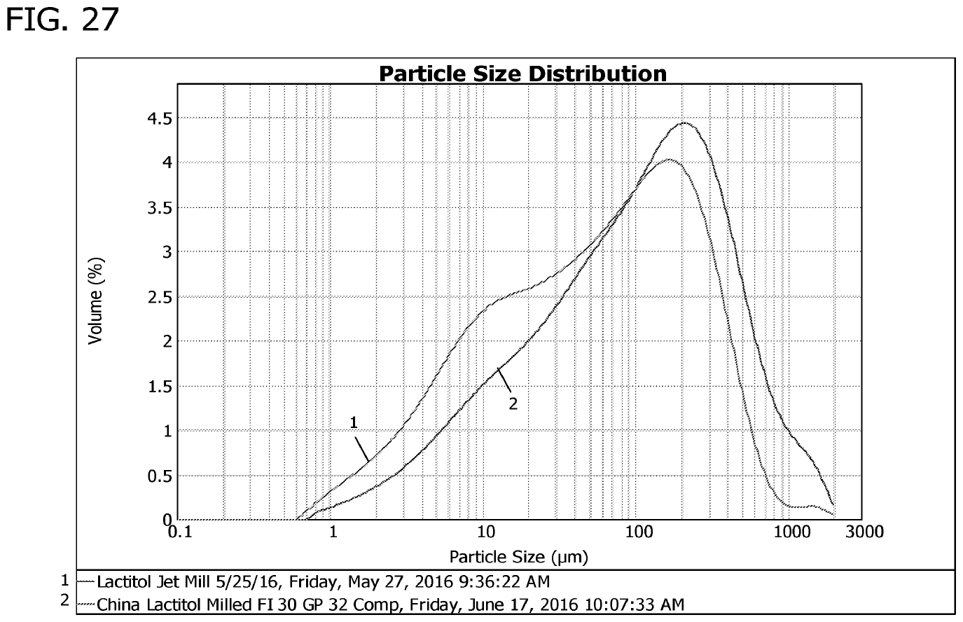

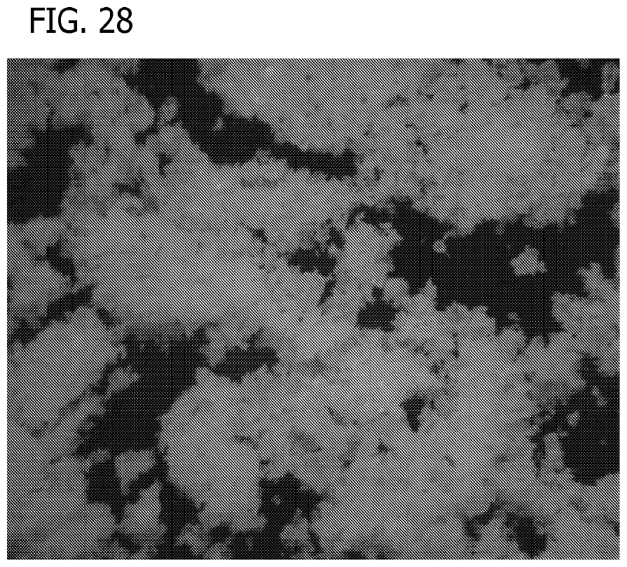

The Lactitol B was jet milled prior to granulating. To do this, 75 Kg (3×25 Kg bags) were jet milled using the same procedure as described above. Three samples were taken over the course of the milling run and composited. The composite sample was evaluated for particle size, bulk density, and tapped density and compared to the jet milled A material previously characterized. The results are displayed in Table 12 and FIG. 27. Test data for the Lactitol A is also included for comparison. A photo micrograph of the milled B material was also generated (See FIG. 28).

TABLE 12
Comparison of Jet Milled Lactitol from two sources

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Jet | 0.0 | 1.5 | 27.5 | 25.3 | 13.8 | 7.5 | 7.1 | 7.4 | 6.6 | 109.3 | 513.4 | — | 0.776 | 0.955 |
| Milled | | | | | | | | | | | | | | |
| A | | | | | | | | | | | | | | |
| Jet | 0.0 | 0.0 | 1.9 | 14.4 | 17.2 | 10.7 | 36.3 | 19.5 | 5.3 | 60.0 | 316.2 | — | 0.670 | 0.933 |
| Milled | | | | | | | | | | | | | | |
| B | | | | | | | | | | | | | | |

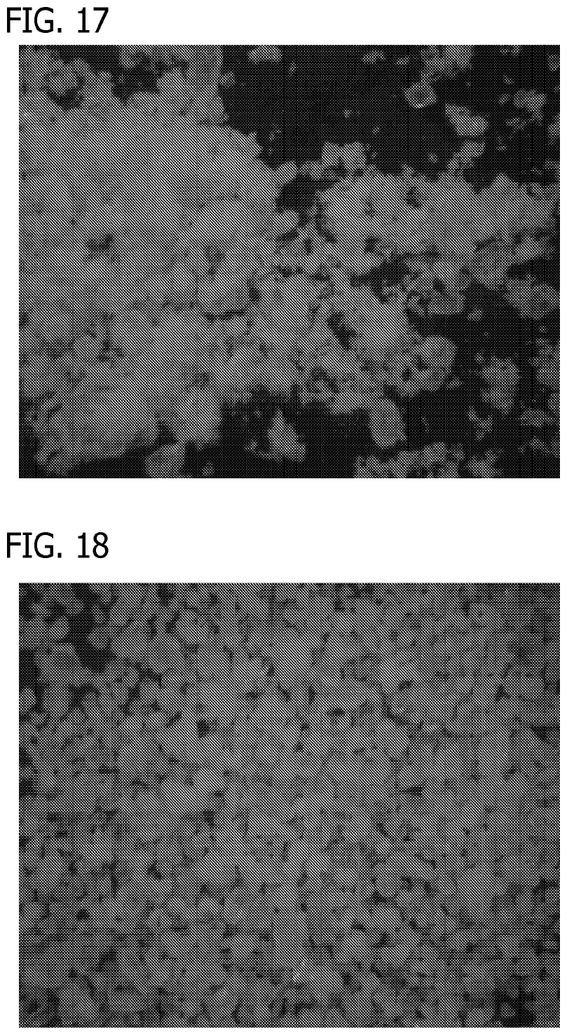

Jet milling the B Lactitol using the same jet mill operating conditions previously used for the A Lactitol resulted in a finer particle size as shown in Table 12 and FIG. 17. This was not totally unexpected as the particle size of the raw Lactitol B was finer than the raw A material. The d50 for the jet milled B Lactitol was 60 microns, compared to a d50 of approximately 109 microns for the A jet mill material used in granulation. Based on FIG. 28, there are not as many un-milled crystals and agglomerates visible compared to the jet milled A lactitol (see FIG. 17). However, the jet milled B material is still within the desired d50 range of approximately 50-100 microns. It was also shown that the bulk density of the jet milled B Lactitol was slightly lower than the bulk density of the jet milled A Lactitol; however, the tapped densities were similar.

Granulation of Jet Milled Lactitol B

To evaluate the suitability of using the jet milled Lactitol B in the Lactitol granulation application, a granulation trial was performed. The final binder temperature was 20° C.

With the granulator running at full speed, the jet milled lactitol feed rate was initiated at 1700 g/min and the binder feed rate was initiated at ˜147 g/min (˜33 Hz). The expected moisture level at this setting would be approximately 4%. At these initial conditions, the granulation exiting the granulator was wetter than desired and clumped together when squeezed. To improve the granulation quality, the binder flow rate was systematically decreased until a granulation of acceptable texture and feel was produced. The final setting was 30 Hz or approximately 134 g/min, which is equivalent to a moisture addition of approximately 3.6%. At this setting a granulation of acceptable texture and feel was produced (˜14 kg). The material produced at these settings was labeled as Drum #1 (sample 13). The binder pump speed was reduced to 28 Hz or approximately 125 g/min and a second drum of granulation (˜14 kg) was produced. This binder addition rate is equivalent to a moisture addition of approximately 3.4%. The material produced at these settings was labeled as Drum #2 (sample 14). The total processing time was approximately 25 minutes. The final wet granulation moisture was approximately 2.3% for both drums produced using a modified Halogen moisture balance LOD method. The modified LOD method included a sample size of approximately 5 g, a temperature of 110° C., and a test duration of 15 minutes.

The wet granulation from each drum was dried (inlet temperature: ˜58° C.; outlet temperature: 49-50° C.). After drying, the sublots from each drum were recombined into separate poly lined drums. A total of 13.3 kg of “as dried” product was recovered from sample 13 and 14.9 kg was recovered from sample 14. The moisture results of the “as dried” samples were approximately 1.5%. The “as dried” material from each sample was then sieved through a 24 TBC screen using a vibratory sieve. For sample 13, a total of 8.5 Kg passed though the screen and 4.8 kg was collected as oversize. For sample 14, a total of 12.0 kg passed through the screen and 2.7 kg was collected as oversize (˜18.4% oversize).

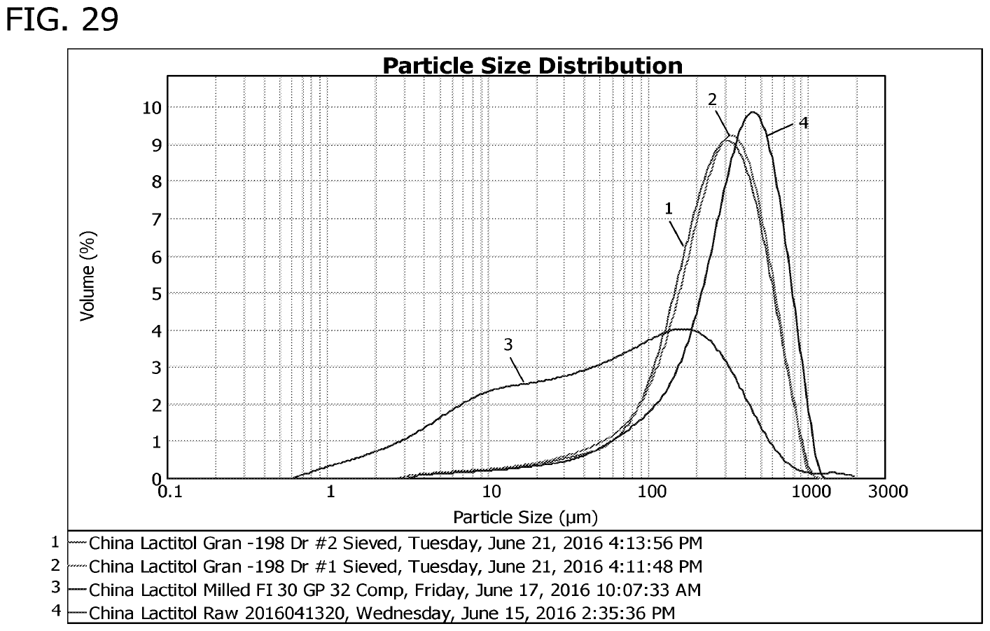

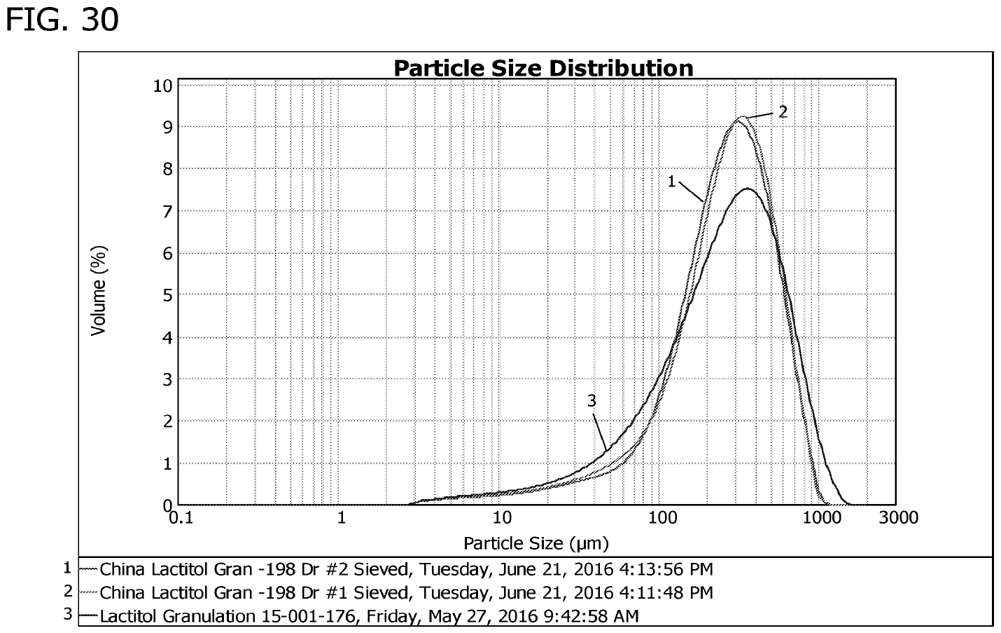

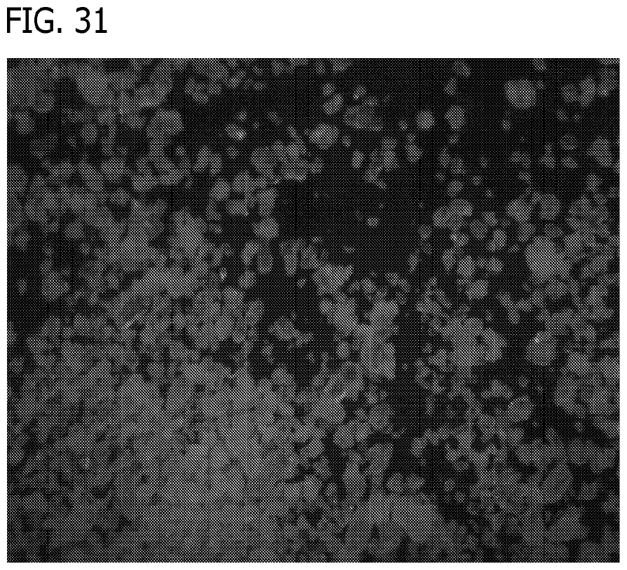

Granulation samples from each drum were tested for particle size, moisture, bulk and tapped density and compared to the previously tested raw Lactitol B and jet milled Lactitol B. The results are displayed in Table 13 and FIG. 29. The particle size results generated here from samples 13 and 14 also were compared to granulation containing jet milled Lactitol A (See FIG. 30). A photo micrograph of the granulation produced with the milled Chinese Lactitol material from sample 14 was also generated (See FIG. 31).

TABLE 13
Comparison of Raw Lactitol, Jet Milled Lactitol, and Granulated Lactitol

| | | | | | | | | | | | | Moisture | Bulk | Tapped |
|---|---|---|---|---|---|---|---|---|---|---|---|---|---|---|
| | Particle Size Distribution (% Retained) | | | | | | | | PS (μ) | | | NMT | Density | Density |
| Sample | 12 | 20 | 40 | 60 | 80 | 100 | 200 | Pan | d10 | d50 | d90 | 3.0% | g/mL | g/mL |
| Raw B | 0.0 | 0.3 | 64.4 | 29.9 | 3.6 | 0.7 | 0.8 | 0.3 | 94.2 | 365.3 | 720.3 | 1.28 | 0.677 | 0.754 |
| Jet | 0.0 | 0.0 | 1.9 | 14.4 | 17.2 | 10.7 | 36.3 | 19.5 | 5.3 | 60.0 | 316.2 | — | 0.670 | 0.933 |
| Milled B | | | | | | | | | | | | | | |
| 13 | 0.0 | 0.0 | 24.4 | 44.1 | 16.8 | 4.6 | 8.4 | 1.6 | 78.1 | 278.4 | 581.2 | 1.46 | 0.696 | 0.779 |
| 14 | 0.0 | 0.0 | 24.8 | 47.0 | 17.5 | 4.1 | 5.7 | 0.8 | 86.7 | 271.4 | 577.8 | 1.58 | 0.678 | 0.773 |

As with the jet milled Lactitol A, it was demonstrated that an acceptable free flowing granulation with an acceptable particle size distribution could be produced using the jet milled Lactitol B. Overall, the particle size of the granulation containing the Jet milled Lactitol B was similar to the particle size of the earlier granulation; however, there were some slight differences. With the granulation containing the B material, the amount retained on the 60 mesh screen was approximately 10% higher while the amount on the 100 and 200 mesh screens and the pan were slightly lower. In addition, there were virtually no differences in particle size between samples 13 and 14 despite the binder flow rate for sample 13 granulation being slightly faster than the binder flow rate for sample 14 granulation (147 g/min versus 125 g/min). With regard to density, both the bulk and tapped density of the granulations produced with the jet milled Lactitol B were slightly lower. The earlier granulations containing the A Lactitol displayed a bulk density of approximately 0.75 g/mL and a tapped density of approximately 0.83 g/mL while the granulations produced with the Lactitol from the B source displayed a bulk density of approximately 0.69 g/mL and a tapped density of approximately 0.78 g/mL. This difference in density can be attributed to the higher bulk and tapped associated with the raw A Lactitol compared to the raw B Lactitol (see Table 13).

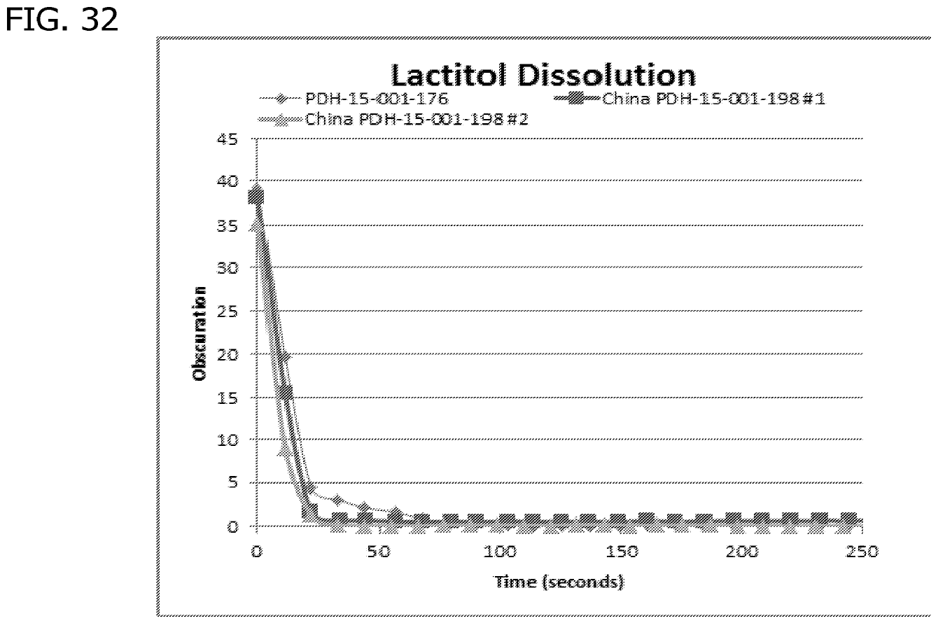

Both lactitol granulations produced above using the Lactitol B (Samples 13 and 14) were tested for dissolution and the results were compared to the results previously generated on granulation Lactitol A. The results are displayed in FIG. 32. The dissolution rate of the two samples 13 and 14 closely matches (no meaningful difference) the dissolution rate of Granulation of Lactitol A.

The above experimental investigation also demonstrates that the Lactitol from a Chinese source (SHANDONG LUJIAN BIOLOGICAL TECHNOLOGY) can be successfully jet milled and granulated using similar granulation processing conditions to those used for the granulation containing the DANISCO lactitol. The finished material after sieving was a free flowing lactitol granulation with only slight differences in particle size and density compared to the granulation produced from the DANISCO lactitol. The granulation produced from the Chinese Lactitol also displayed a dissolution rate, based on the dissolution test method, that was similar to the dissolution rate of the granulation produced from the DANISCO lactitol.

### Example 2: Scale-up of Lactitol Granulation

The following equipment was used unless otherwise indicated: STURTEVANT MICRONIZER Jet Mill, Model 45DM; stainless steel jacketed kettle, 90 gallons (binder preparation tank); LIGHTNIN Air Mixer with propeller blade (binder solution mixer); WAUKESHA, #15 Process Pump, #15962 (binder solution pump); pressure nozzle(s) (SPRAYING SYSTEMS) with spray tip (spray nozzle); ACCURATE Powder Feeder, #9664; HOSAKAWA BEPEX TURBULIZER, Model T14, #09650 (granulator); AEROMATIC Fluid Bed Dryer, 430 L bowl, #6287, #9665; SWECO Vibratory Sieve, 30″. This example investigates the grinding pressure, powder flow rate, binder flow rate, fill amounts, and drying schemes of the processes of the present invention and their effect on a scale-up of the lactitol granulation procedures previously described.

Jet Milling of Raw Lactitol

For this plant trial, 950 kilograms of Lactitol Monohydrate Food Grade were received from SHANDONG LUJIAN BIOLOGICAL TECHNOLOGY CO, LTD, China. The STURTEVANT MICRONIZER Jet Mill, Model 45DM was set up in the plant pharmacy and attached to house compressed air. 50 kg at a time were charged to an ACCURATE feeder. With the outlet of the feeder positioned over the Jet Mill hopper, the raw Lactitol was fed to the jet mill at a steady rate and milled using a grinding pressure between 30-32 psi and a feed injection pressure of approximately 30 psi. Each approximately 50 kilograms of jet milled material (˜50 kg) were collected in double lined 23 gallon fiber drums and labeled. During the milling operation samples of the jet material were taken throughout the run and tested for MALVERN particle size and moisture by Karl Fischer. The target d50 particle size range was 50-100 microns. During the milling process adjustments were made to the grinding pressure to maintain the particle size within the desired range. The production rate for jet milling each 50 kilogram portion ranged from 40-50 minutes. The particle size and moisture results are displayed in Table 14 (rate ˜40-50 minutes per 50 kg milled lactitol; raw lactitol KF moisture—4.93%).

TABLE 14
Effect of Variation of Grinding Pressure

| | Grinding | Feed | | | | | Moisture |
|---|---|---|---|---|---|---|---|
| Drum | Pressure | Injection | Particle Size (μ) | | | | after Mill |
| No. | (PSI) | (PSI) | d10 | d50 | d90 | Span | % KF |
| 1 | 32 | 30 | 4.8 | 44.5 | 241.2 | 5.3 | 4.96 |
| 2 | 32 | 30 | 4.4 | 37.8 | 234.0 | 6.1 | 4.94 |
| 3 | 30 | 30 | 8.3 | 112.5 | 467.4 | 4.1 | 4.94 |
| 5 | 31 | 30 | 6.5 | 81.7 | 381.6 | 4.5 | 4.98 |
| 10 | 31 | 30 | 7.4 | 97.7 | 395.8 | 3.9 | 5.08 |
| 12 | 31 | 30 | 7.7 | 98.6 | 413.0 | 4.1 | 5.13 |
| 14 | 31 | 30 | 7.5 | 93.1 | 391.5 | 4.1 | 5.14 |
| 15 | 31 | 30 | 8.2 | 106.6 | 432.2 | 3.9 | 5.16 |
| 19 | 32 | 30 | 5.4 | 58.2 | 322.8 | 5.5 | 5.12 |
| Average | 31 | 30 | 6.7 | 81.2 | 364.4 | 4.6 | 5.05 |
| Proof of | 32 | 30 | 5.3 | 60.0 | 316.2 | 5.2 | |
| Concept | | | | | | | |

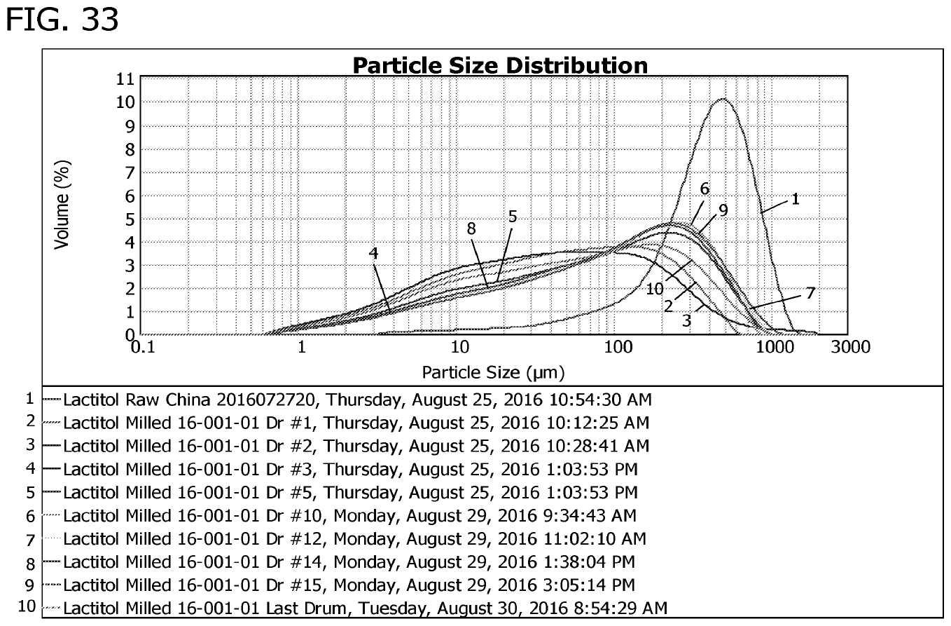

As can be seen above, the average d50 particle size was well within the desired approximately 50 to 100 micron range at 81.2 microns. The particle size curves for each sample tested along with the curve of the raw Lactitol starting material are displayed in FIG. 33.

The average moisture across all samples was 5.05%, just slightly above the moisture of the raw Lactitol. A total of 934.4 kilograms of jet milled material was recovered. The jet milling operation yield was 98.4%. See Table 15.

TABLE 15
Weight Jet Milled Lactitol Recovered

| Drum No. | Weight (kg) | Theo. Weight (kg) |
|---|---|---|
| 1-5 | 246.0 | 250.0 |
| 6 | 48.8 | 50.0 |
| 7 | 49.0 | 50.0 |
| 8 | 48.8 | 50.0 |
| 9 | 49.5 | 50.0 |
| 10 | 48.6 | 50.0 |
| 11 | 48.4 | 50.0 |
| 12 | 50.0 | 50.0 |
| 13 | 49.4 | 50.0 |
| 14 | 49.4 | 50.0 |
| 15 | 49.4 | 50.0 |
| 16 | 49.7 | 50.0 |
| 17 | 48.9 | 50.0 |
| 18 | 50.3 | 50.0 |
| 19 | 48.2 | 50.0 |
| Total | 993.4 | 950.0 |

| | |
|---|---|
| Yield | 98.36 |

Granulation of Jet Milled Lactitol

The formulation of lactitol as described in Table 16 was used.

TABLE 16
Dry Ingredients

| Ingredient | Quantity (g/min) | % | Purpose |
|---|---|---|---|
| Lactitol (Jet Milled Crystal) | 9500.0 | 97.37 | Dry Feed |
| Lactitol (in Binder Solution) | 257.0 | 2.63 | Binder |
| Lactitol Granulation Output | 9756.6 | 100.00 | |

The amount of lactitol in the binder solution was 42%. The target granulation moisture was 3.5%. This percentage equates to a water addition rate of 354.9 g/minute. Based on these requirements, a binder solution was prepared according to Table 17.

TABLE 17
Binder Solution for Desired Moisture of 3.5%

| Ingredient | Quantity (g/min) | % | Batch Amount (Kg) |
|---|---|---|---|
| Lactitol (Raw Crystal) | 257.0 | 42.0 | 75.0 |
| Potable Water | 354.9 | 58.0 | 103.6 |
| Needed Binder Output | 611.9 | 100 | 178.6 |

Approximately 250 kg of USP Purified water was added to a 250 gal stainless steel jacketed tank. The water was heated to 30° C. Once heated, 104 kg of heated water was siphoned over to a 90 gallon stainless steel tank equipped with a mixer. After the mixer was started and an adequate vortex was obtained, 75 kilograms of raw lactitol was added to the tank and mixed. The resultant binder solution was mixed for 15 minutes. At the completion of the 15 minute mix time, some undissolved lactitol had accumulated near the drain valve of the tank. Using a plastic spatula the undissolved lactitol was further agitated while mixing to ensure all of the lactitol was dissolved. Once all of the material was visibly dissolved, mixing was discontinued. The final temperature of the binder solution after mixing was 18° C.

Using the above binder solution, ten tubs of Lactitol Granulation were produced using various powder flow rates, binder flow rate, and different tub fill amount, and different drying schemes. All material was passed through a vibratory sieve fitted with a 14 TBC upper screen and a 24 TBC lower screen. The material passing the through the 24 TBC screen was packaged as good material in 23 gallon, double lined drums. The material passing through the 14 TBC screen but retained on the 24 TBC was directed to the Jet mill. The jet milled material was transferred back to the top of the vibratory sieve for further processing. The material retained on the 14 TBC screen was collected in a drum as oversize. Samples from various points of the process were taken throughout the run and analyzed.

The operating conditions used for each of the ten tubs produced during this scale-up trial are summarized in Table 18. For all granulation trials, two, 0.060″ (No. 8) spray nozzles were used. The granulator impeller speed was set at 1225 rpm to maintain the same impeller tip speed as that used on the granulator in Example 1. Two spray nozzles were used for each trial and the spray tip size was 0.060 inches. The moisture of the milled lactitol prior to granulation was 5.05%.

TABLE 18
Granulation Conditions

| | Powder | | Binder | Binder | | | | |
|---|---|---|---|---|---|---|---|---|
| | Auger | Powder | Solution | Solution | Binder | Tub | Wet Granulation | |
| Sample | Setting | Flow Rate | Setting | Pressure | Flow Rate | Loading | Moisture | |
| Tub | Report | (kg/min) | (rpm) | (psi) | (g/min) | (kg) | % KF | % Gain |
| 1 | 850 | 9.2 | 975 | 18 | 596 | 71 | 7.89 | 2.84 |
| 2 | 850 | 9.2 | 975 | 18 | 596 | 82 | 8.25 | 3.20 |
| 3 | 950 | 11.0 | 1105 | 17 | 710 | 71 | 8.35 | 3.30 |
| 4 | 950 | 11.0 | 1045 | 18 | 710 | 71 | 7.78 | 2.73 |
| 5 | 950 | 11.0 | 1045 | 18 | 710 | 72 | 7.80 | 2.75 |
| 6 | 950 | 11.0 | 1045 | 18 | 710 | 82 | 7.85 | 280 |
| 7 | 950 | 11.0 | 1045 | 17 | 710 | 90 | 8.13 | 3.08 |
| 8 | 950 | 11.0 | 1045 | 18 | 710 | 100 | 7.48 | 2.43 |
| 9 | 950 | 11.0 | 1045 | 18 | 710 | 100 | 7.90 | 2.85 |
| 10 | 995 | 13.0 | 1200 | 18 | 840 | 100 | 7.93 | 2.88 |

At the target conditions of samples 1 and 2, the expected wet granulation moisture would be approximately 3.5%. At these initial conditions, a granulation of acceptable texture and feel was produced. The average wet granulation moisture for these two samples was approximately 3%. For granulation samples 4-9, the powder flow rate was increased to 11 kg/min (660 Kg/hr) and the binder flow rate was increased to maintain the wet granulation moisture at approximately 3%. At these conditions the process continued to produce a granulation of acceptable texture and feel and the granulator handled the higher flow rates without any issues. During the manufacture of these samples, the amount of granulation collected in each sample tub was varied to determine optimal loading for maintaining acceptable granulation characteristics and acceptable production rates and to help determine the optimal fill for the drying process. The sample tubs were successfully filled up to 100 kg per tub without issue. For granulation Sample Tub 10, the powder feed rate was adjusted to the maximum for the current feeder system. This resulted in the powder flow rate of 13 Kg/min (780 Kg/hr). The binder flow rate was once again increased to maintain the wet granulation moisture at approximately 3%. At this maximum powder feed rate, the process continued to produce a granulation of acceptable texture and feel. The granulator was able to handle the maximum powder feed rate and subsequent increase in the binder flow rate without issue.

Fluid Bed Drying of the Lactitol Granulations

After each sample tub of granulation was produced as described above, they were dried in the fluid bed dryers (inlet temperature: 60° C. due to low glass transition of lactitol). However difficulties were encountered controlling the dryer inlet at this temperature throughout the scale-up run. The steam controllers on the fluid bed dryers are inadequate to maintain the temperature consistently at the lower temperatures required for this product.

During the first 3 minute 15 second drying of sample tub 1, the inlet temperature ranged from 63 to 81° C. After the drying time expired, the outlet temperature was 49° C. However, the material was still not completely dry based on visual observations. Drying continued for an additional 2 minutes with the inlet temperature ranging between 73 and 82° C. before the majority of the water added through the binder addition was evaporated. For sample tubs 2 and 3, the actual inlet temperature was approximately 83° C. Sample tub 2 was initially dried for 5 minutes then for approximately 4 additional minutes while sample tub 3 was dried for a total of 4 minutes total. The dried material in both tubs contained numerous lumps that were likely formed due the higher than desired inlet temperatures used during the drying process. The material closest to the tub walls and bottom screen was agglomerated the worst in both cases.

Since the inlet temperature of fluid bed dryers could not be successfully controlled at the desired target temperature (60° C.), and the actual inlet temperatures achieved during processing were variable and significantly higher than desired resulting in significant product agglomeration, it was decided to investigate the possibility of first drying the granulation in one of the fluid bed dryers (dryer 2) with the heat off to partially dry and fluff up the material before exposing the material to a final heated dry in the other oven (dryer 1). This should help minimize sticking of the granulation to the tub walls and bottom screens that occurs at higher drying temperatures. To investigate this, the granulation in sample tub 4 was dried (Dryer 2) with no heat for 10 minutes. The inlet temperature was 23.5° C. After the 10 minute dry time, the contents of the tub were mixed by hand with a large plastic spatula. Very few lumps were observed in the product. The material still had a wet appearance and was cool to the touch. Next, the tub was dried for an additional 10 minutes with no heat. The contents of the tub were again mixed by hand with a large plastic spatula after the second 10 minute dry time. The product was still relatively lump-free but still looked wet. To do the final drying, the tub was dried in the other dryer (Dryer 1) with the inlet temperature set at approximately 55° C. The material was dried until a dryer outlet temperature of 56° C. was achieved. After the heated dry, the material appeared to be dry and free flowing and was relatively lump free. The initial moisture and moisture results determined after each drying step are displayed in Table 19.

Based on the above results the use of an unheated drying cycle is beneficial for minimizing agglomeration of the product that frequently occurs when it is exposed to high inlet temperatures. When the lactitol granulation is at its wettest stage, the higher temperatures tend to flash dry the product resulting in lumps that are hard on the outside and do not break up easily during subsequent processing. This leads to higher amounts of oversize material that ultimately affects product yield. It is also been shown that a short heated dry cycle is still needed after the unheated drying cycle to complete the drying of the product. Granulations that have not been exposed to a short heated dry cycle (curing step) that drives off the remainder of the unbound moisture added during the granulation process do not exhibit the same desired physical characteristics for finished lactitol granulation.

The remainder of the granulation sublots, sample tubs 5-10, utilized this two-step drying procedure. The unheated dry time was decreased with the remaining sublots to improve process throughput. Sample tubs 5 and 6 used a single 10 minute unheated dry time and sample tubs 7-10 used an 8 minute unheated dry time. All tubs were stirred with a plastic spatula before moving to the heated drying phase to break up any lumps and loosen any material sticking to the side of the tub or screen. The heated dry time was determined by monitoring the outlet temperature of the dryer. The target outlet temperature was 50° C. During the heated dry time for Tubs 5-10 the inlet temperature ranged from approximately 47-70° C. as inlet temperature control remained an issue. The overall time for the dryer to reach a 50° C. outlet temperature ranged from 5-8 minutes. The dry time did not appear to be significantly affected by the amount of wet granulation in the tubs. The amount in the tubs ranged from approximately 70 kg to 100 kg. After the final heated dry cycle, the dried granulations were visually inspected. Based on visual observation, the use of the unheated dry cycle before the heated dry cycle was effective in minimizing the lumping, product sticking to the side of the tub, and agglomeration that were previously observed when only heated drying was utilized.

Table 19 displays the moisture data collected during these trials.

TABLE 19
Scale-up moisture results

| | Wet | | | | | | | | | |
|---|---|---|---|---|---|---|---|---|---|---|
| | Granulation | | | | | | | Amount | | |
| | Moisture | | Moisture after Unheated | | Moisture after | | | unbound in Left | | Moisture |
| Sample | % | | Drying | | Heated Drying | | Total | Granulation | | after Sieve |
| Tub | KF | % Gain | % KF | % Loss | % KF | % Loss | % Loss | % KF | % KF | % Gain/Loss |
| 1 | 7.89 | 2.84 | — | — | 5.19 | 2.70 | 2.70 | 0.14 | — | — |
| 2 | 8.25 | 3.20 | — | — | 5.00 | 3.25 | 3.25 | 0.00 | 5.05 | 0.05 |
| 3 | 8.35 | 3.30 | — | — | 5.37 | .98 | 2.98 | 0.32 | 5.48 | 0.11 |
| 4 | 7.78 | 2.73 | 5.91% at 10 min | 1.87 | 5.00 | 0.91 | 3.56 | 0.00 | 5.06 | 0.06 |
| | | | 5.13% at 20 min | 2.65 | | | | | | |
| 5 | 7.80 | 2.75 | 5.67% at 10 min | 2.13 | 5.23 | 0.44 | 2.57 | 0.18 | 5.21 | −0.13 |
| 6 | 7.85 | 2.80 | 7.16% at 10 min | 0.69 | .93 | 2.23 | 2.92 | 0.00 | 5.10 | 0.17 |
| 7 | 8.13 | 3.08 | 6.82% at 10 min | 1.31 | 5.21 | 1.16 | 2.47 | 0.61 | 5.23 | 0.02 |
| 8 | 7.48 | 2.43 | 6.78% at 8 min | 0.70 | 5.23 | 1.55 | 2.25 | 0.18 | 5.24 | 0.01 |
| 9 | 7.90 | 2.85 | 5.80% at 8 min | 2.10 | 5.03 | 0.77 | 2.87 | 0.00 | 5.17 | 0.14 |
| 10 | 7.93 | 2.88 | 6.08% at 8 min | 1.85 | 5.32 | 0.76 | 2.61 | 0.27 | 5.21 | −0.11 |
| Avg. | 7.94 | 2.89 | — | 1.46 | 5.15 | 1.68 | 2.82 | 0.17 | 5.19 | 0.04 |

Sieving/Milling of the Lactitol Granulations

The sieving operation utilized a 30″ vibratory sieve fitted with 14 TBC top screen and a 24 TBC bottom screen. The material on the 14 TBC screen was collected in a drum as oversize. The material collected on the 24 TBC screen was directed to the jet mill (grinding pressure: 40 psi; feed injection pressure: 40 psi). The jet milled material was transferred back to the top of the vibratory sieve for further processing. All material passing through the 24 TBC screen was packaged as finished product in 23 gallon fiber pack drums with two poly liners. The amount of finished product collected and the amount of oversize material collected for each tub are displayed in Table 20.

TABLE 20
Results of Sieving/Milling Lactitol Granulations

| | Tub Loading | | Net Dry Wt. per | Total Net Wt. | Total Oversize |
|---|---|---|---|---|---|
| Sample Tub | (wet) (kg) | Drum No. | drum (kg) | (Drums 1 & 2) (kg) | Weight (kg) |
| 1 | 71.0 | 1 | 48.0 | 59.9 | 2.7 |
| | | 2 | 9.2 | | |
| 2 | 82.0 | 1 | 41.0 | 41.0 | 43.8 |
| | | 2 | — | | |
| 3 | 71.0 | 1 | 39.4 | 39.4 | 21.9 |
| | | 2 | — | | |
| 4 | 71.0 | 1 | 51.4 | 61.4 | 1.3 |
| | | 2 | 10.0 | | |
| 5 | 72.0 | 1 | 50.0 | 61.5 | 8.3 |
| | | 2 | 11.5 | | |
| 6 | 82.0 | 1 | 50.0 | 71.4 | 6.1 |
| | | 2 | 21.4 | | |
| 7 | 90.0 | 1 | 51.8 | 67.9 | 2.7 |
| | | 2 | 16.1 | | |
| 8 | 100.0 | 1 | 50.7 | 88.8 | 4.9 |
| | | 2 | 38.1 | | |
| 9 | 100.0 | 1 | 50.5 | 95.6 | 7.2 |
| | | 2 | 45.1 | | |
| 10 | 100.0 | 1 | 50.1 | 88.1 | 6.7 |
| | | 2 | 38.0 | | |

As can be seen in Table 20, the use of an unheated dry cycle along with a short heated drying cycle was effective in reducing the amount of oversize produced compared to using heated drying alone. Sample tubs 2 and 3 that utilized only heated drying had significantly more oversize than sample tubs 4-10 that utilized the unheated dry cycle and a short heated dry cycle.

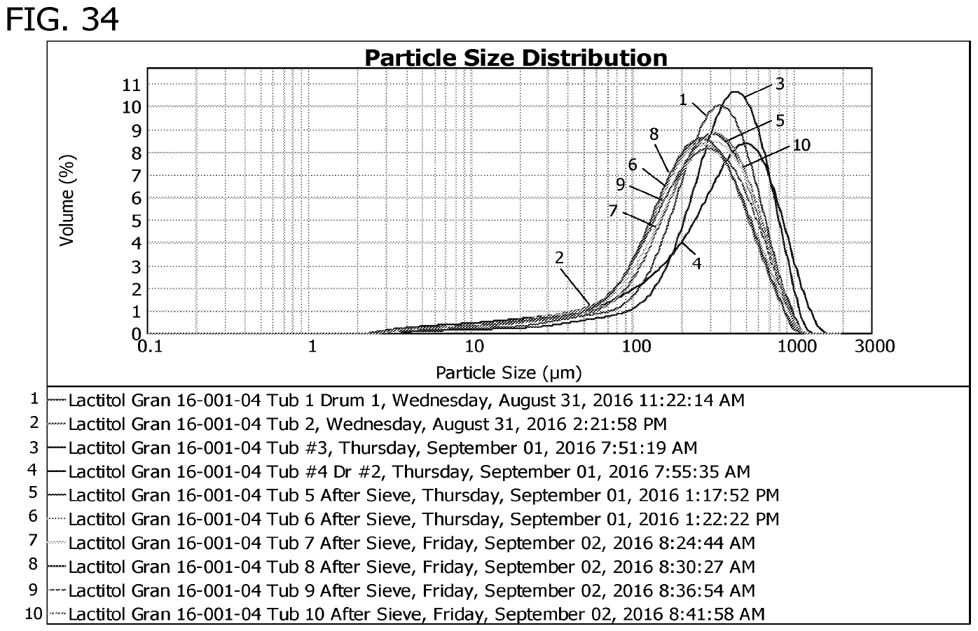

Before and during the sieving/milling process for each tub (sublot) samples were taken and analyzed for particle size, bulk and tapped density, and moisture. The results are displayed in Table 21 and FIG. 34. Table 22 compares the particle size after drying versus after sieving for Tubs 5-10 that utilized both the cool drying cycle and the final heated drying cycle. The percentage change due to the sieving/milling operation is also summarized.

TABLE 21
Scale-up Particle Size, Density, and Moisture Results.

| | Particle Size | | | | | | | | | | Bulk | Tapped | Moisture |
|---|---|---|---|---|---|---|---|---|---|---|---|---|---|
| Sample | Distribution (% Retained) | | | | | | | PS (μ) (after sieve) | | | Density | Density | after Sieve |
| Tub | 12 | 20 | 40 | 60 | 100 | 200 | Pan | d10 | d50 | d90 | g/mL | g/mL | % KF |
| 1 as dried | 1.6 | 6.3 | 50.5 | 37.4 | 4.1 | 0.2 | 0.0 | — | — | — | 0.671 | 0.793 | — |
| 1 | 0.0 | 0.1 | 44.0 | 42.9 | 10.0 | 2.4 | 0.6 | 101.2 | 307.7 | 612.5 | 0.676 | 0.820 | — |
| 2 | 0.0 | 0.2 | 43.5 | 38.1 | 13.7 | 3.6 | 1.1 | 55.5 | 233.2 | 528.3 | 0.659 | 0.794 | 5.05 |
| 3 | 0.0 | 0.1 | 49.4 | 37.6 | 10.5 | 2.1 | 0.3 | 137.8 | 381.9 | 725.4 | 0.667 | 0.794 | 5.48 |
| 4, Drum 1 | 0.0 | 0.2 | 29.6 | 48.1 | 18.5 | 3.0 | 0.6 | 106.1 | 328.0 | 657.9 | 0.725 | 0.862 | 5.06 |
| 4, Drum 2 | 0.0 | 0.3 | 36.3 | 26.0 | 18.2 | 11.7 | 7.5 | 56.1 | 310.9 | 705.5 | 0.735 | 0.943 | 5.13 |
| 5 | 0.0 | 0.0 | 22.2 | 40.3 | 27.1 | 8.1 | 2.3 | 74.3 | 272.4 | 598.8 | 0.735 | 0.893 | 5.21 |
| 6 | 0.0 | 0.2 | 24.8 | 36.7 | 27.5 | 8.7 | 2.1 | 63.8 | 239.6 | 556.1 | 0.735 | 0.862 | 5.10 |
| 7 | 0.0 | 0..2 | 19.5 | 39.8 | 30.5 | 8.3 | 1.7 | 74.2 | 267.0 | 594.9 | 0.735 | 0.862 | 5.23 |
| 8 as dried | 1.1 | 3.2 | 22.8 | 36.9 | 27.2 | 8.1 | 0.8 | 89.2 | 285.9 | 632.3 | 0.704 | 0.833 | — |
| 8 | 0.0 | 0.2 | 19.8 | 39.8 | 29.3 | 9..0 | 2.0 | 66.4 | 236.1 | 533.5 | 0.714 | 0.877 | 5.24 |
| 9 | 0.0 | 0.1 | 24.7 | 36.7 | 27.0 | 9.5 | 2.0 | 68.6 | 250.5 | 578.6 | 0.704 | 0.862 | 5.17 |
| 10 | 0.0 | 0.1 | 20.6 | 40.3 | 28.8 | 8.1 | 2.2 | 79.1 | 276.4 | 598.2 | 0.725 | 0.877 | 5.21 |

TABLE 22
Scale-up Particle Size after Drying vs. After Sieving/Milling

| | | Particle Size (μ) | | | | % Change due to | | |
|---|---|---|---|---|---|---|---|---|
| Sample | | (after sieve/milling) | | | | Sieving/Milling | | |
| Tub | | d10 | d50 | d90 | Span | d10 | d50 | d90 |
| 5 | After Dry | 127.3 | 351.2 | 708.7 | 1.7 | −41.6 | −22.4 | −15.5 |
| | After | 74.3 | 272.4 | 598.8 | 1.7 | | | |
| | Sieving/ | | | | | | | |
| | Milling | | | | | | | |
| 6 | After Dry | 108.0 | 309.8 | 638.6 | 1.7 | −40.9 | −22.7 | −12.9 |
| | After | 63.8 | 239.6 | 556.1 | 2.0 | | | |
| | Sieving/ | | | | | | | |
| | Milling | | | | | | | |
| 7 | After Dry | 98.4 | 312.4 | 687.9 | 1.9 | −24.6 | −14.5 | −13.5 |
| | After | 74.2 | 267.0 | 594.9 | 2.0 | | | |
| | Sieving/ | | | | | | | |
| | Milling | | | | | | | |
| 8 | After Dry | 89.2 | 285.9 | 632.3 | 1.9 | −25.6 | −17.4 | −15.6 |
| | After | 66.4 | 236.1 | 533.5 | 2.0 | | | |
| | Sieving/ | | | | | | | |
| | Milling | | | | | | | |
| 9 | After Dry | 79.7 | 271.4 | 629.1 | 2.0 | −13.9 | −7.7 | −8.0 |
| | After | 68.6 | 250.5 | 578.6 | 2.0 | | | |
| | Sieving/ | | | | | | | |
| | Milling | | | | | | | |
| 10 | After Dry | 59.7 | 202.8 | 572.9 | 2.5 | 32.5 | 36.3 | 4.4 |
| | After | 79.1 | 276.4 | 598.2 | 1.9 | | | |
| | Sieving/ | | | | | | | |
| | Milling | | | | | | | |

As can be seen in Table 21, the overall particle size of each granulation sublot were more consistent after the two step unheated/heated drying process was implemented (Sample tubs 5-10) and the bulk and tapped density was consistently higher (>0.85 g/mL).

The particle size did not appear to be affected by the tub loading. Sample tub 5 was charged with 72 kg of wet granulation, sample tub 25 was charged with 82 Kg of wet granulation, sample tub 7 was charged with ˜90 Kg of wet granulation, and sample tubs 8-10 were charged with 100 kg of wet granulation. It was also shown that the amount of wet granulation collected in each tub had very little impact on the final product tapped density. The tapped density for sample tubs 5-10 ranged from 0.833 g/mL to 0.893 g/mL with an average of 0.867 g/mL. The current density target is NMT 0.82 g/mL.

The particle size after drying and the particle size after milling/sieving were compared as displayed in Table 22. The sieving and milling operation significantly decreased the overall particle size of the “as dried” material as indicated by the percentage change for the d10, d50, and d90 results. As the experiential progressed from sample tub 5 though sample tub 9, better control of the drying parameters were achieved that resulted in a smaller “as dried” particle size. Therefore the percent change in particle size of the latter Tubs was lower. It appears that there was a sampling error associated with sample tub 10 as the particle size after sieving and milling was larger than the particle size of the “as dried” the particle size results for sample tub 10. The samples may be been misidentified.

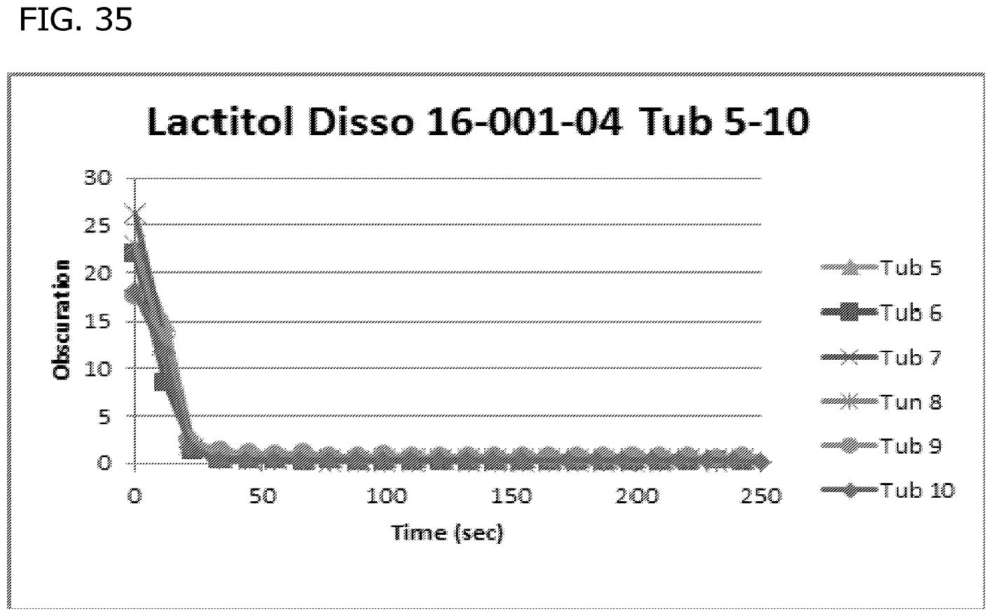

Samples of the material produced from sample tubs 5-10 were also tested for dissolution rate using the wet sampling attachment for the MALVERN MASTERSIZER 2000. The results are displayed in FIG. 35. As shown, the dissolution results of sample tubs 5-10 were similar and comparable to previous results from R&D lab scale granulation trials.

Through execution of this scale-up trial, it was shown that jet milled lactitol with a d50 particle size of approximately 50-100 microns could be successfully granulated using a granulator with two spray nozzles. A total of ten granulation sublots (Tubs) were successfully produced using a range of operating conditions (powder flow rates, tub fill amounts, one and two step drying processes etc.) to determine potential production rates for commercial production. It was shown that powder flow rate from approximately 9.5-13 kilograms per minute could be achieved without significantly impacting the wet granulation physical characteristics. This translates to a wet granulation rate of approximately 570-780 per hour.

Initially a single heated drying step was evaluated that utilized a 60° C. inlet temperature, however, difficulties in maintaining control of the inlet temperature in the fluid bed dryers (higher temperatures than desired during processing) resulted in the production of a significant amount of oversize. At higher temperatures, the granulation tends to flash dry causing case hardening and the formulation of hard lumps that do not breakup easily. To combat these issues, a two-step drying process was successfully implemented. First an unheated drying cycle was used to partially drying the granulation to minimize agglomeration of the product that frequently occurs when drying the material at higher inlet temperatures. An eight (8) minute unheated dry time was established. The partially dried granulations were then further dried in a heated fluid bed dryer (˜60° C.) until an outlet temperature of approximately 50-55° C. was achieved. It was shown that this final heated drying step (curing step) was needed to drive off the remainder of the unbound moisture added during the granulation process and to allow the finished product to achieve its desired physical characteristics (flowability, particle size and density etc.) The heated drying time needed to achieve an outlet temperature in the range of 50-55° C. was approximately 4.5-9 minutes.

With the two step unheated/heated drying process in place, the overall particle size of each granulation sublot (tub) after sieving and milling of the oversize were more consistent and the bulk and tapped density was consistently higher (>0.85 g/mL). Dissolution results on granulation samples collected during execution of this scale-up trial were similar and comparable to previous results from the lab scale granulation trials.

### Example 3: Optimization of Scale-up of Lactitol Granulation

This Example seeks to identify the optimal parameters of a scaled-up production of granulated lactitol. This Example investigates the effects of grinding pressure, granulator speed, granulation moisture content, powder feed rate/maximum production rate, dryer outlet temperature, consecutive granulation runs, and the addition of raw lactitol to milled lactitol. This Example also considers flowability and drying parameters.

Jet Milling of Crystal Lactitol Monohydrate, NF/FCC

For this commercial optimization trial, approximately 1950 kilograms (78, 25 kg bag) of crystal Lactitol Monohydrate NF/FCC received from SHANDONG LUJIAN BIOLOGICAL TECHNOLOGY CO, LTD, China were jet milled. The STURTEVANT MICRONIZER Jet Mill, Model 45DM was set up in the plant pharmacy and attached to house compressed air. Two bags at a time (50 kg) were charged to an ACCURATE feeder. With the outlet of the feeder positioned over the Jet Mill hopper, the raw lactitol was fed to the jet mill at a steady rate and milled using a grinding pressure between 26-32 psi and a feed injection pressure of approximately 30 psi. Each approximately 50 kilograms of jet milled material was collected in double lined 23 gallon fiber drums and labeled. During the milling operation samples of the jet milled material were taken throughout the run using a sample thief and tested for Malvern particle size. During the milling process, adjustments were made to the grinding pressure to maintain the particle size within the desired range. The production time for jet milling each 50 kilogram portion ranged from 45-50 minutes (60-67 Kg/hour production rate). MALVERN particle size results are displayed in Table 23.

TABLE 23
Particle size of Sample Drums

| | Grinding | | | | | |
|---|---|---|---|---|---|---|
| Drum | Pressure | Feed Injection | Particle size (μ) | | | |
| No. | (PSI) | Pressure (PSI) | d10 | d50 | d90 | Span |
| 1 | 30 | 30 | 4.7 | 51.9 | 361.2 | 6.9 |
| 2 | 30 | 30 | 5.1 | 55.5 | 340.8 | 6.0 |
| 3 | 30 | 30 | 5.2 | 54.5 | 297.3 | 5.4 |
| 5 | 30 | 30 | 5.4 | 57.8 | 331.5 | 5.6 |
| 9 | 30 | 30 | 4.2 | 38.6 | 265.5 | 6.8 |
| 13 | 30 | 30 | 4.0 | 31.4 | 271.1 | 8.5 |
| 17 | 30 | 30 | 4.0 | 37.5 | 287.9 | 7.6 |
| 20 | 28 | 30 | 4.0 | 33.8 | 209.8 | 6.1 |
| 21 | 28 | 30 | 5.0 | 50.8 | 290.2 | 5.6 |
| 22 | 28 | 30 | 4.4 | 36.7 | 235.3 | 6.3 |
| 24 | 28 | 30 | 5.4 | 53.1 | 289.0 | 5.3 |
| 25 | 28 | 30 | 4.2 | 35.5 | 261.8 | 7.3 |
| 26 | 26 | 30 | 4.4 | 40.8 | 298.4 | 7.2 |
| 27 | 26 | 30 | 4.2 | 40.2 | 272.3 | '6.7 |
| 28 | 26 | 30 | 4.6 | 40.4 | 263.7 | 6.4 |
| 29 | 26 | 30 | 5.7 | 84.8 | 401.6 | 4.7 |
| 33 | 26 | 30 | 4.1 | 36.6 | 267.6 | 7.2 |
| 37 | 26 | 30 | 4.4 | 43.9 | 337.2 | 7.6 |
| Last | 26 | 30 | 4.2 | 39.1 | 341.6 | 8.6 |
| Average | 28 | 30 | 4.6 | 45.4 | 296.0 | 6.6 |

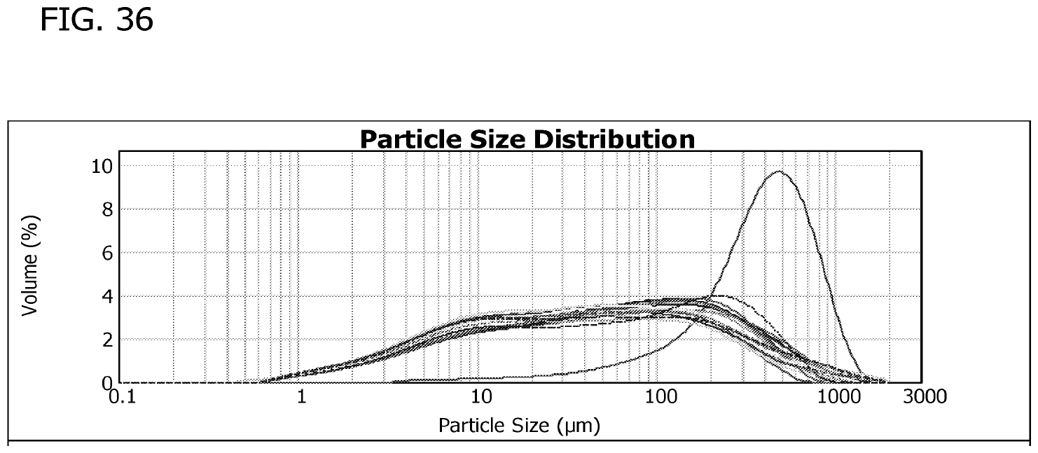

As can be seen, the average d50 particle size of the jet milled lactitol was approximately 45 microns. The particle size curves for each sample tested along with the curve of the raw lactitol starting material are displayed in FIG. 36. For informational purposes, the moisture level of the milled lactitol was also determined to be 4.99% based on KF analysis. A total of 1937.0 kilograms of jet milled material was recovered. The jet milling usable yield was 99.3%. The total weight of each drum is reported in Table 24.

TABLE 24
Total drum weight

| Drum No. | Weight (kg) |
|---|---|
| 1 | 48.3 |
| 2 | 49.7 |
| 3 | 49.2 |
| 4 | 49.8 |
| 5 | 49.7 |
| 6 | 50.3 |
| 7 | 50.0 |
| 8 | 49.7 |
| 9 | 50.1 |
| 10 | 49.6 |
| 11 | 50.2 |
| 12 | 49.9 |
| 13 | 49.1 |
| 14 | 49.5 |
| 15 | 49.2 |
| 16 | 48.9 |
| 17 | 49.4 |
| 18 | 49.2 |
| 19 | 49.9 |
| 20 | 49.6 |
| 21 | 49.9 |
| 22 | 49.5 |
| 23 | 50.0 |
| 84 | 50.0 |
| 25 | 49.7 |
| 26 | 49.9 |
| 27 | 49.8 |
| 28 | 49.5 |
| 29 | 50.2 |
| 30 | 49.7 |
| 31 | 49.6 |
| 32 | 49.9 |
| 33 | 50.0 |
| 34 | 50.2 |
| 35 | 49.6 |
| 36 | 48.6 |
| 37 | 49.6 |
| 38 | 49.9 |
| Last (39) | 50.1 |
| Total | 1937.0 |
| Usable yield (1937/195 × 100) | 99.3 |

Binder Makeup

A total of 325 kilograms (42%) of crystal lactitol monohydrate NF/FCC was dissolved in 448.8 kilograms (58%) of Purified Water. The total amount per binder sublot is 773.8 kilograms. During the binder preparation procedures, the mixing speed and mixing time needed to adequately disperse the crystal lactitol monohydrate were quantitatively defined and documented. Visual observation during processing was utilized to establish the appropriate mixing speed and mixing time, reported in Table 25. Once an appropriate mixing speed was set, the actual speed was determined using a calibrated, hand held tachometer and recorded on the batch record. The crystal lactitol monohydrate was added and mixed at the set speed until it was completely dissolved. The time the crystal lactitol monohydrate was completely dissolved was noted as the minimum mixing time

TABLE 25
Mixer Settings

| | Mixer | Mixing | |
|---|---|---|---|
| | Speed | Speed | Mix Time |
| Binder Make-Up Step No. | Setting | (RPM) | (Minutes) |
| Step 3 - Addition and | 90% | 289 | 15 |
| mixing of Item 1-2: | | | |
| Crystal Lactitol Monohydrate | | | |
| Step 4 - Mixing of Finished | 40% | 100 | Continuous |
| Lactitol Binder Solution | | | During |
| | | | Processing |

For Step 3, the target mixing speed was rounded to 290 rpm. A ±10 rpm range was applied to this speed to allow for any equipment variability. Therefore, the mixing speed range for the lactitol solution preparation will be set at 280-300 rpm. The minimum mixing time for Step 3 will be set at 15 minutes. For Step 4, the target mixing speed will be 100 rpm with a range of 90-110 rpm. Since the mixing speeds and time were established for the worst case, crystal lactitol (coarser, harder to dissolve), the mixing speeds and mixing time would also be appropriate for using milled lactitol to prepare the lactitol binder solution.

Granulation, Drying, Sieving, Milling

The formulation of Table 26 was the target formulation for this test.

TABLE 26
Dry Ingredients

| Ingredient | Quantity (g/min) | % | Purpose |
|---|---|---|---|
| Lactitol (Jet Milled Crystal) | 11000 | 97.72 | Dry Feed |
| Lactitol (in Binder Solution) | 257 | 2.28 | Binder |
| Lactitol Granulation Output | 11,257 | 100.00 | |

The target wet granulation moisture for the lactitol granulation is 3.06%. This percentage equates to a water addition rate of 354.9 grams/minute. Based on this water addition rate and the required addition rate for the lactitol in the binder solution (from Table 27) the following target binder solution will be needed for the lactitol granulation.

TABLE 27
Binder Solution for Desired Moisture of 3.06%

| | Quantity | | Batch |
|---|---|---|---|
| Ingredient | (g/min) | % | Amount (Kg) |
| Lactitol (Raw Crystal) | 257.0 | 42.0 | 325.0 |
| Potable Water | 354.9 | 58.0 | 448.8 |
| Needed Binder Output | 611.9 | 100 | 773.8 |

Utilizing a lactitol binder solution as described in Table 27 and the target formulation feed rate as a starting point, a series of optimization trials were produced to evaluate the effects of granulator speed, high granulation moisture, low granulation moisture, high production rate, and drying conditions on the lactitol granulation proposed critical quality attributes. Particle size, tapped density, and moisture testing were utilized to assess the effect of these process parameters on the lactitol granulation and to establish appropriate control strategies for the process parameters considered critical. For tub 17, approximately 10% raw, unmilled lactitol was mixed with the milled lactitol prior to granulation.

For each trial, approximately 100 kilograms of wet granulation were produced at the specified granulation conditions and collected in a single drying tub. Two spray nozzles with 0.060″ spray tips were used to deliver the lactitol solution to the granulator. During the granulation process, a sample of the wet granulation was taken and tested for moisture by Karl Fischer. Drying of the wet granulation was accomplished using a two-step drying process. The wet granulation was first dried in an ambient temperature (˜25° C.) fluid bed dryer to partially dry and fluff up the granulation. Following the ambient temperature drying cycle, drying of the wet granulation was completed in a heated fluid bed dryer (inlet temperature: 50-70° C.; outlet temperature: 50-55° C.). Tub 8 was dried until the lower outlet temperature was reached; tub 9 was dried until the upper outlet temperature was reached. For tubs 12-13, the inlet temperature controller was adjusted from 25° C. to zero, and the steam was turned completely off. For each tub produced, a sample “as dried” material was taken as directed in the master batch record and immediately tested for KF moisture. Drying was complete when a moisture of NMT 5.5% was achieved. An additional sample of the “as dried” material was taken for particle size (ROTAP and MALVERN) and tapped density testing. Drying parameters are summarized in Table 28A.

Next, each fluid bed drying tub containing the “as dried” granulation was inverted over a vibratory sieve fitted with a #14 TBC top screen, and a #24 TBC bottom screen. Material on the top screen was collected as oversize material. Material between the #14 TBC top screen and #24 TBC bottom screen was directed to a jet mill and returned to the top of the sieve. The sieved material from each tub was collected in double lined 23 gallon fiber board drums according to the master batch record. During the sieving/milling process, in-process samples of the finished granulation were taken and tested for particle size and tapped density testing. An additional sample of the finished product was taken for MALVERN particle size analysis as well as flowability testing. Parameters for each tub are listed in Table 28.

TABLE 28
Variation of Parameters

| | | Powder | Powder | Binder | Binder | Binder | Spray | No. of | |
|---|---|---|---|---|---|---|---|---|---|
| | Granulator | Auger | Feed | Solution | Solution | Feed | Tip | Spray | Tub |
| Tub | Speed | Setting | Rate | Setting | Pressure | Rate | Size | Nozzles | Loading |
| No. | (rpm) | Report | (Kg/min) | (rpm) | (psi) | (g/min) | (inches) | Used | (Kg) |
| 1 | 1227 | 965 | 10.4 | 1051 | 12 | 604 | 0.060 | 2 | 100 |
| 2 | 1227 | 965 | 10.4 | 1051 | 12 | 604 | 0.060 | 2 | 100 |
| 3 | 1450 | 965 | 10.4 | 1051 | 12 | 604 | 0.060 | 2 | 100 |
| 4 | 1000 | 965 | 10.4 | 1051 | 12 | 604 | 0.060 | 2 | 100 |
| 5 | 1227 | 875 | 9.4 | 1351 | 18 | 759 | 0.060 | 2 | 100 |
| 6 | 1227 | 999 | 12.4 | 701 | 2 | 454 | 0.060 | 2 | 100 |
| 7 | 1227 | 999 | 12.4 | 1351 | 18 | 759 | 0.060 | 2 | 100 |
| 8 | 1227 | 965 | 10.4 | 1051 | 12 | 604 | 0.060 | 2 | 100 |
| 9 | 1227 | 965 | 10.4 | 1051 | 12 | 604 | 0.060 | 2 | 100 |
| 12 | 1227 | 965 | 10.4 | 1051 | 12 | 604 | 0.060 | 2 | 100 |
| 13 | 1227 | 965 | 10.4 | 1051 | 12 | 604 | 0.060 | 2 | 100 |
| 14 | 1227 | 965 | 10.4 | 1102 | 14 | 632 | 0.060 | 2 | 100 |
| 15 | 1227 | 965 | 10.4 | 1149 | 15 | 660 | 0.060 | 2 | 100 |
| 16 | 1227 | 965 | 10.4 | 1102 | 14 | 632 | 0.060 | 2 | 100 |
| 17 | 1227 | 965 | 10.4 | 1102 | 14 | 632 | 0.060 | 2 | 100 |

After granulation, both tubs of wet granulation were dried using the two step drying process and sieved using the procedures detailed in the Example 2, specific parameters listed in Table 28A.

TABLE 28A
Drying Parameters

| | | | | | | In Process |
|---|---|---|---|---|---|---|
| | | | | | | Moisture |
| | Total | Inlet T | Inlet T | Outlet T | Exhaust | (After Heated |
| | Drying | Ambient | Heated Fluid | Heated Fluid | Blower Speed | Drying) |
| | Time | Fluid Bed | Bed | Bed | (Heated FB) | NMT |
| Tub No. | (Minutes) | Report | 50-70° C. | 50-55° C. | 1200-1700 | 5.5%(KF) |
| 1 | 18 | 27 | 60 | 54 | 1650 | 5.1 |
| 2 | 17 | 26 | 64 | 53 | 1650 | 5.0 |
| 3 | 20 | 27 | 66 | 54 | 1650 | 5.1 |
| 4 | 18 | 27 | 61 | 55 | 1650 | 5.1 |
| 5 | 22 | 23 | 61 | 55 | 1650 | 5.0 |
| 6 | 20 | 25 | 65 | 55 | 1650 | 5.1 |
| 7 | 24 | 27 | 64 | 55 | 1650 | 5.1 |
| 8 | 18 | 32 | 68 | 50 | 1650 | 5.1 |
| 9 | 18 | 33 | 68 | 56 | 1650 | 5.1 |
| 10 | 18 | 33 | 75 | 55 | 1650 | 5.1 |
| 11 | 20 | 35 | 72 | 62 | 1650 | 5.0 |
| 12 | 18 | 23 | 70 | 55 | 1650 | 5.0 |
| 13 | 21 | 27 | 69 | 55 | 1650 | 5.0 |
| 14 | 18 | 30 | 76 | 53 | 1650 | 5.0 |
| 15 | 19 | 33 | 76 | 53 | 1650 | 5.1 |
| 16 | 18 | 39 | 76 | 53 | 1650 | 5.0 |
| 17 | 20 | 39 | 77 | 53 | 1650 | 5.1 |
| Avg. | 19 | 30 | 69 | 54 | 1650 | 5.1 |
| Min. | 17 | 23 | 60 | 50 | 1650 | 5.0 |
| Max. | 24 | 39 | 77 | 62 | 1650 | 5.1 |
| s | 1.82 | 5.01 | 5.82 | 2.40 | 0.00 | 0.05 |

As shown above, the average total drying time which includes the ambient drying cycle time, tub mixing and transferring to the heated fluid bed dryer, and the final heating drying cycle time was 19 minutes across all tubs produced.

The inlet temperature on the ambient fluid bed dryer was monitored throughout the execution of the commercial optimization protocol. At the beginning of the study the inlet temperature controller of the ambient fluid bed dryer was set at 25° C. To maintain this temperature, the steam valves were adjusted to approximately 3% open. Through the first seven tubs, the inlet temperature remained within the range of 23-27° C., but started to creep higher as the commercial optimization trial continued. Despite eventually turning off the steam to the ambient fluid bed dryer completely before processing Tub 12, the inlet temperature still was higher than desired. It was hypothesized that even with the steam valves off, steam was still bleeding through the valves.

Control of the inlet temperature within the range of 50-70° C. during the heated drying cycle was acceptable while processing the first 9 tubs. For the subsequent tubs, the inlet temperature exceeded the upper limit of this range with the inlet temperatures ranging from 70 to 77° C. Difficulties in maintaining control of the inlet temperature were also experienced during the scale-up run. Previous studies have shown that high inlet temperatures during the heated drying cycle result in particle size variation and the production of a significant amount of oversize material. It is recommended that adjustments to the fluid bed dryer controls be made to ensure that the desired temperature range (50-70° C.) is maintained. The data also shows that using the 50-55° C. temperature endpoints for the heated drying cycle is effective in producing a Lactitol granulation that consistently meets the final granulation moisture requirements of NMT 5.5% (KF).

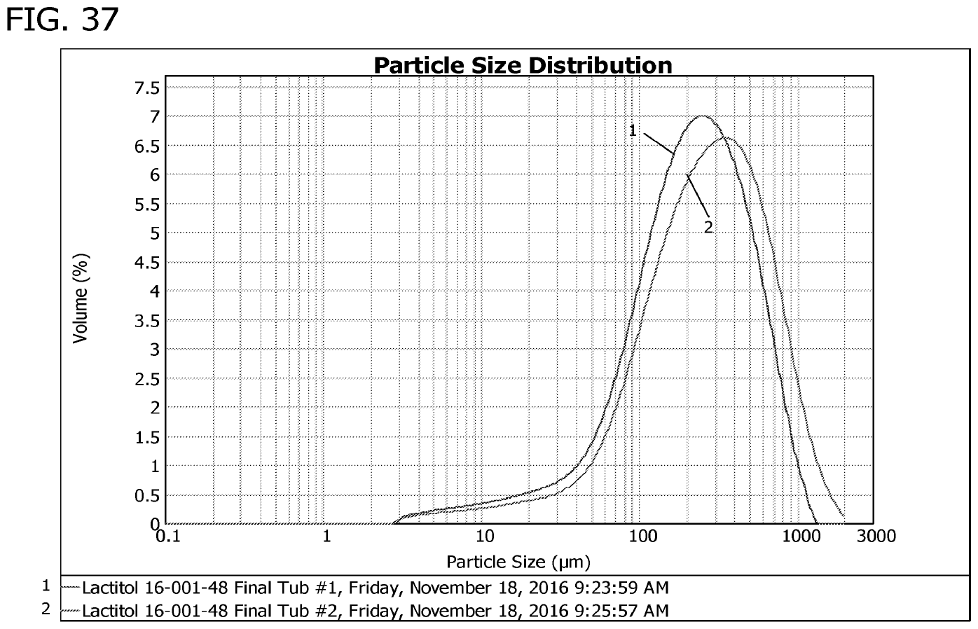

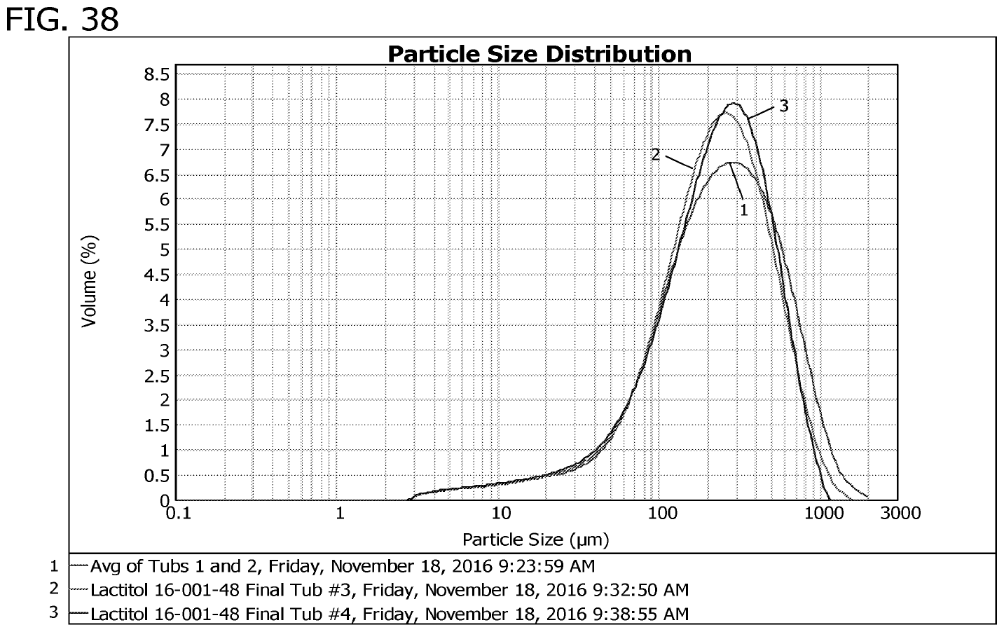

Samples taken during the granulation, drying, and sieving processes were tested per the commercial optimization protocol and the master batch record. The results are displayed in Tables 29-31 and FIGS. 37-42. The amounts produced from each tub, the amount of oversize, and the total material recovered is summarized in Table 32.

TABLE 29
Moisture Results

| | Wet | | | | | | | | |
|---|---|---|---|---|---|---|---|---|---|
| | Granulation | | | | Moisture after | | | Unbound | |
| | Moisture | | Moisture after | | Heated Drying | | | Water Left in | Moisture |
| | 7-9.5 | | Unheated Drying | | NMT | | Total | Granulation | after Sieve |
| Sample | % KF | % Gain | % KF | % Loss | 5.5% | % Loss | % Loss | % KF | % KF |
| 1 | 7.70 | 2.71 | 5.83 | 1.87 | 5.1 | 0.73 | 2.60 | 0.11 | 5.04 |
| 2 | 7.98 | 2.99 | — | — | 5.0 | — | 2.98 | 0.01 | 5.04 |
| 3 | 7.51 | 2.52 | 5.89 | 1.62 | 5.1 | 0.79 | 2.41 | 0.11 | 5.03 |
| 4 | 7.60 | 2.61 | 5.38 | 2.22 | 5.1 | 0.28 | 2.500 | 0.11 | 5.04 |
| 5 | 9.08 | 4.09 | 7.95 | 1.13 | 5.0 | 2.95 | 4.08 | 0.01 | 5.07 |
| 6 | 6.77 | 1.78 | 5.24 | 1.53 | 5.1 | 0.14 | 1.67 | 0.11 | 5.07 |
| 7 | 8.09 | 3.10 | 7.24 | 0.85 | 5.1 | 2.14 | 2.99 | 0.11 | 5.02 |
| 8 | 7.76 | 2.77 | 5.88 | 1.88 | 5.1 | 0.78 | 2.66 | 0.11 | 5.07 |
| 9 | 7.52 | 2.53 | 5.90 | 1.62 | 5.1 | 0.80 | 2.42 | 0.11 | 5.09 |
| 12 | 7.99 | 3.00 | 6.89 | 1.10 | 5.0 | 1.89 | 2.99 | 0.01 | 5.08 |
| 13 | 7.43 | 2.44 | 6.55 | 0.88 | 5.0 | 2.43 | 2.43 | 0.01 | 5.09 |
| 14 | 7.70 | 2.71 | 5.71 | 1.99 | 5.0 | 2.70 | 2.70 | 0.01 | 5.07 |
| 15 | 7.94 | 2.95 | 6.94 | 1.00 | 5.1 | 1.84 | 2.84 | 0.11 | 5.08 |
| 16 | 7.32 | 2.33 | 5.33 | 1.99 | 5.0 | 0.33 | 2.32 | 0.01 | 5.07 |

TABLE 30
Particle Size Results, Tubs 1-9 and 17

| | | | | | | | | | | | | | Tapped |
|---|---|---|---|---|---|---|---|---|---|---|---|---|---|
| | | | | | | | | | | | | | Density |
| | | Particle Size Distribution (% retained) | | | | | | | | | | Bulk | g/mL |
| | | | 20 | 40 | | 100 | | Pan | PS (μ) (after sieve) | | | Density | NLT |
| | | 12 | NMT | NMT | 60 | NLT | 200 | NMT | d10 | d50 | d90 | g/mL | 0.82 |
| Sample | State* | Report | 1% | 35% | Report | 18% | Report | 5% | Report | Report | Report | Report | g/mL |
| 1 | AD | 0.0 | 2.5 | 20.5 | 24.5 | 31.5 | 18.8 | 2.2 | 64.9 | 240.9 | 685.1 | 0.76 | 0.83 |
| | FP | 0.0 | 0.0 | 14.7 | 26.5 | 32.4 | 23.3 | 3.1 | 85.2 | 219.9 | 588.3 | 0.67 | 0.85 |
| 2 | AD | 1.3 | 5.2 | 24.0 | 29.8 | 30.2 | 8.3 | 0.5 | 83.9 | 282.3 | 756.9 | 0.71 | 0.83 |
| | FP | 0.0 | 0.0 | 21.4 | 22.1 | 27.9 | 22.6 | 6.0 | 73.2 | 277.4 | 773.6 | 0.75 | 0.85 |
| 3 | AD | 0.5 | 2.9 | 16.5 | 30.7 | 33.3 | 14.2 | 1.9 | 68.2 | 243.5 | 609.9 | 0.71 | 0.82 |
| | FP | 0.0 | 0.0 | 12.9 | 30.6 | 33.6 | 20.1 | 2.9 | 62.9 | 226.4 | 566.9 | 0.70 | 0.83 |
| 4 | AD | — | — | — | — | — | — | — | — | — | — | — | — |
| | FP | 0.0 | 0.0 | 12.0 | 35.0 | 34.2 | 16.1 | 2.7 | 59.9 | 2333 | 52.1 | 0.69 | 0.83 |
| 5 | AD | 3.0 | 7.2 | 25.5 | 40.6 | 19.9 | 3.1 | 0.7 | 109.5 | 300.6 | 647.5 | 0.71 | 0.78 |
| | FP | 0.0 | 0.0 | 25.6 | 49.0 | 19.3 | 4.6 | 1.5 | 103.0 | 308.0 | 612.1 | 0.64 | 0.77 |
| 6 | AD | 0.0 | 0.3 | 3.1 | 9.9 | 26.4 | 45.6 | 14.8 | 30.3 | 116.9 | 385.0 | 0.63 | 0.75 |
| | FP | 0.0 | 0.0 | 4.8 | 21.9 | 33.6 | 33.3 | 6.5 | 37. | 164.7 | 423.0 | 0.68 | 0.83 |
| 7 | AD | 1.6 | 4.3 | 18.7 | 41.0 | 27.6 | 5.7 | 1.1 | 77.7 | 260.0 | 571.4 | 0.68 | 0.78 |
| | FP | 0.0 | 0.0 | 11.6 | 43.3 | 32.9 | 10.1 | 2.1 | 67.5 | 225.0 | 470.2 | 0.63 | 0.77 |
| 8 | AD | 1.0 | 3.3 | 15.2 | 37.5 | 34.0 | 8.2 | 1.1 | 75.4 | 244.8 | 536.2 | 0.66 | 0.78 |
| | FP | 0.0 | 0.0 | 7.2 | 36.7 | 37.9 | 15.5 | 2.7 | 58.9 | 206.9 | 460.3 | 0.64 | 0.77 |
| 9 | AD | 0.1 | 2.0 | 12.1 | 31.8 | 37.7 | 14.3 | 2.0 | 64.2 | 230.0 | 538.8 | 0.68 | 0.79 |
| | FP | 0.0 | 0.0 | 7.5 | 29.4 | 36.9 | 22.9 | 3.3 | 48.3 | 186.8 | 466.8 | 0.65 | 0.78 |
| 17 | AD | 0.9 | 2.7 | 31.5 | 37.3 | 22.1 | 4.5 | 1.1 | 101.1 | 308.0 | 684.7 | 0.68 | 0.79 |
| | FP | 0.0 | 0.0 | 26.5 | 43.4 | 21.8 | 6.6 | 1.7 | 87.1 | 282.8 | 607. | 0.67 | 0.79 |

*AD = As Dried, FP = Finished Product

TABLE 31
Particle Size Results, Consecutive Run Tubs 12-16

| | | | | | | | | | | | | | Tapped |
|---|---|---|---|---|---|---|---|---|---|---|---|---|---|
| | | | | | | | | | | | | | Density |
| | | Particle Size Distribution (% retained) | | | | | | | PS (μ) (after sieve) | | | Bulk | g/mL |
| | | | 20 | 40 | | 100 | | Pan | | | | Density | NLT |
| | | 12 | NMT | NMT | 60 | NLT | 200 | NMT | d10 | d50 | d90 | g/mL | 0.82 |
| Sample | State * | Report | 1% | 35% | Report | 18% | Report | 5% | Report | Report | Report | Report | g/mL |
| 12 | AD | 0.2 | 2.4 | 19.2 | 34.4 | 33.1 | 9.2 | 1.5 | 72.0 | 253.2 | 613.0 | 0.71 | 0.85 |
| | FP | 0.0 | 0.0 | 17.3 | 41.7 | 32.0 | 8.4 | 0.7 | 79.5 | 245.3 | 550.9 | 0.68 | 0.82 |
| 13 | AD | 0.4 | 2.8 | 26.7 | 36.6 | 27.1 | 5.7 | 0.7 | 93.2 | 300.3 | 691.2 | 0.70 | 0.83 |
| | FP | 0.0 | 0.0 | 19.3 | 40.5 | 30.6 | 8.7 | 0.9 | 78.7 | 248.8 | 553.2 | 0.68 | 0.82 |
| 14 | AD | 0.4 | 3.1 | 26.2 | 31.7 | 25.4 | 11.3 | 1.9 | 79.3 | 274.9 | 651.3 | 0.71 | 0.83 |
| | FP | 0.0 | 0.0 | 26.1 | 40.2 | 24.0 | 8.2 | 1.5 | 84.2 | 297.2 | 669.8 | 0.69 | 0.83 |
| 15 | AD | 1.2 | 4.3 | 33.5 | 334.3 | 20.4 | 5.4 | 0.9 | 101.6 | 320.9 | 746.1 | 0.69 | 0.82 |
| | FP | 0.0 | 0.0 | 32.7 | 40.0 | 20.4 | 5.7 | 1.2 | 73.2 | 259.8 | 593.5 | 0.69 | 0.83 |
| 16 | AD | 0.5 | 2.5 | 26.9 | 33.9 | 26.2 | 8.7 | 1.4 | 87.4 | 291.0 | 700.3 | 0.72 | 0.83 |
| | FP | 0.0 | 0.0 | 24.5 | 34.1 | 25.1 | 13.2 | 3.1 | 65.3 | 275.9 | 699.1 | 0.70 | 0.85 |
| Avg. | AD | 0.5 | 3.0 | 26.5 | 34.2 | 26.4 | 8.1 | 1.3 | 86.7 | 288.0 | 680.4 | 0.71 | 0.83 |
| | FP | 0.0 | 0.0 | 24.0 | 39.3 | 26.4 | 8.8 | 1.5 | 76.2 | 264.8 | 613.3 | 0.69 | 0.83 |
| Min. | AD | 0.2 | 2.4 | 19.2 | 31.7 | 20.4 | 5.4 | 0.7 | 72.0 | 253.2 | 613.0 | 0.7 | 0.82 |
| | FP | 0.0 | 0.0 | 17.3 | 34.1 | 20.4 | 5.7 | 0.7 | 65.3 | 245.3 | 550.9 | 0.7 | 0.82 |
| Max. | AD | 1.2 | 4.3 | 33.5 | 36.6 | 33.1 | 11.3 | 1.9 | 101.6 | 320.9 | 746.1 | 0.7 | 0.85 |
| | FP | 0.0 | 0.0 | 32.7 | 41.7 | 32.0 | 13.2 | 3.1 | 84.2 | 297.2 | 699.1 | 0.7 | 0.85 |
| s | AD | 0.38 | 0.77 | 5.06 | 1.74 | 4.54 | 2.49 | 0.48 | 11.57 | 25.63 | 50.53 | 0.01 | 0.01 |
| | FP | 0.00 | 0.00 | 6.07 | 2.98 | 4.81 | 2.72 | 0.95 | 7.23 | 22.02 | 67.92 | 0.01 | 0.01 |
| RSD | AD | 0.00 | 0.00 | 19.11 | 5.10 | 17.17 | 30.93 | 37.63 | 13.35 | 8.90 | 7.43 | 1.61 | 1.32 |
| | FP | 0.00 | 0.00 | 25.31 | 7.58 | 18.20 | 30.72 | 64.53 | 9.49 | 8.32 | 11.07 | 1.22 | 1.48 |

* AD = As Dried, FP = Finished Product

Tubs 1 and 2:

As can be seen above, the in-process wet granulation moistures for both tubs 1 and 2 were just slightly below the target of 3.06% with the binder flow rate and powder feed rate near their respective target settings. The final moisture for both tubs 1 and 2 was below the NMT 5.5% limit using the proposed two step drying process. The tapped density of the finished product from each tub 1 and 2 was within the proposed finished product specifications. The ROTAP particle results were all within the proposed specification except for Tub 2. The amount retained in the receiving pan was slightly higher at 6% than the original proposed 5% limit.

Tubs 3 and 4:

The granulations representing the high and low granulator speeds for tubs 3 and 4 both had similar appearance and texture to the granulations produced at the target impeller speed (Tubs 1 & 2). The in-process wet granulation moistures for both tubs were just slightly below the target at approximately 2.6% with the binder flow rate and powder feed rate near their respective target settings. The final moisture for both tubs 3 and 4 was below the NMT 5.5% limit using the proposed two step drying process. The speed of the granulator did not appear to have a significant impact on the amount of oversize material produced. Whether the granulator speed was at the target setting or at the upper and lower speeds tested, the amount of oversize produced was in the 1-2% range. As can be seen in Table 30, the particle size and tapped density results after sieving for each tub were within the proposed finished product specifications. Overall the data shows the granulator speed within the range tested has no impact on final moisture and minimal impact on product particle size and tapped density. Based on these results the granulator impeller speed should not be considered a critical process parameter.

Tubs 5 and 6:

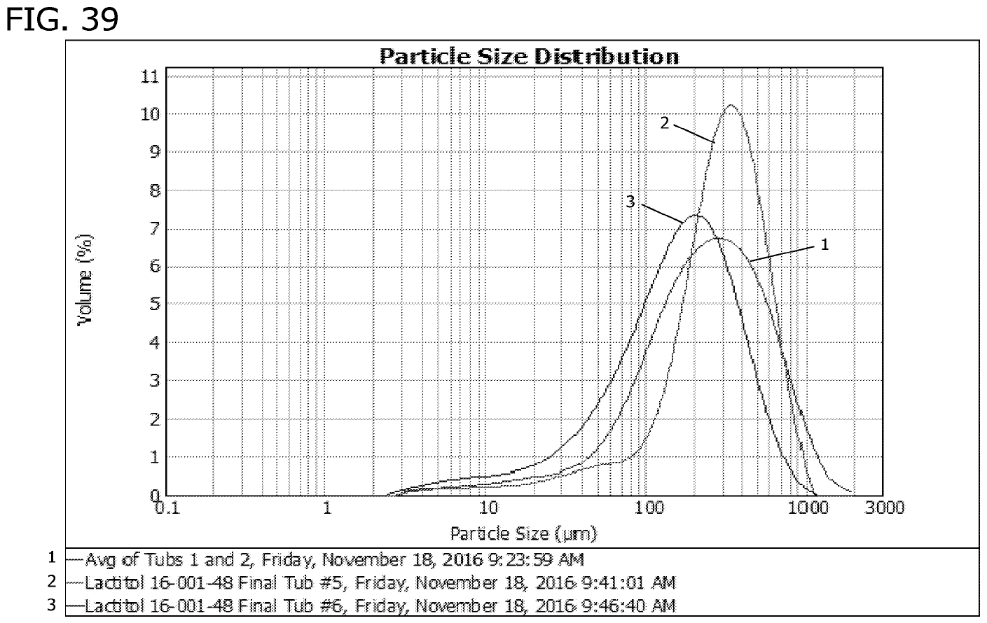

As expected, the granulation moisture of Tub #5 was near the upper end of the currently proposed wet granulation moisture at 9.08%. This is equivalent to a 4.09% moisture gain during granulation. While the ROTAP particle size results met the proposed limits for the Lactitol granulation, the particle size distribution was shifted to the coarser end of the range as evident by the increase amounts retained on the 40 and 60 mesh screens as compared to the granulations produced at target conditions (Tubs 1 & 2) and reduced amounts of finer material on the 100 mesh screen and in the receiving pan. The shift in particle size is also depicted with the MALVERN particle size results in FIG. 39. The amount of oversize material (plus 14 TBC screen) produced at the higher wet granulation moisture condition was also significantly higher than the granulation produced at target conditions (˜11% versus 1-3%). At the high moisture granulation conditions used here, the tapped density of the finished granulation did not meet the minimum of 0.82 g/mL. The shift in particle size may be responsible for the lower tapped density.

As expected, the granulation moisture of Tub #6 was slightly below the lower end of the currently proposed wet granulation moisture at 6.67%. This is equivalent to a 1.78% moisture gain during granulation. Compared to the granulations produced under the target conditions (Tubs 1 & 2), the particle size distribution shifted to the finer end of the proposed range at lower moisture conditions as evident by the significantly reduced amount on the 40 mesh screen with corresponding increase of finer material on the 200 mesh screen. As with the second tub produced at target conditions, the amount in the receiving pan was slightly higher than the proposed limit of NMT 5%. The shift in particle size due to the lower moisture conditions is also depicted with the MALVERN particle size results in FIG. 39. The amount of oversize material (plus 14 TBC screen) produced at the lower wet granulation moisture condition was slightly lower than the granulation produced at target conditions (0.9% versus 1-3%). At the low moisture granulation conditions evaluated here, the proposed tapped density requirement of NLT 0.82 g/mL for the finished granulation was met. Overall the data shows the wet granulation moisture within the range tested does impact the particle size and the tapped density of the finished product.

Tub 7:

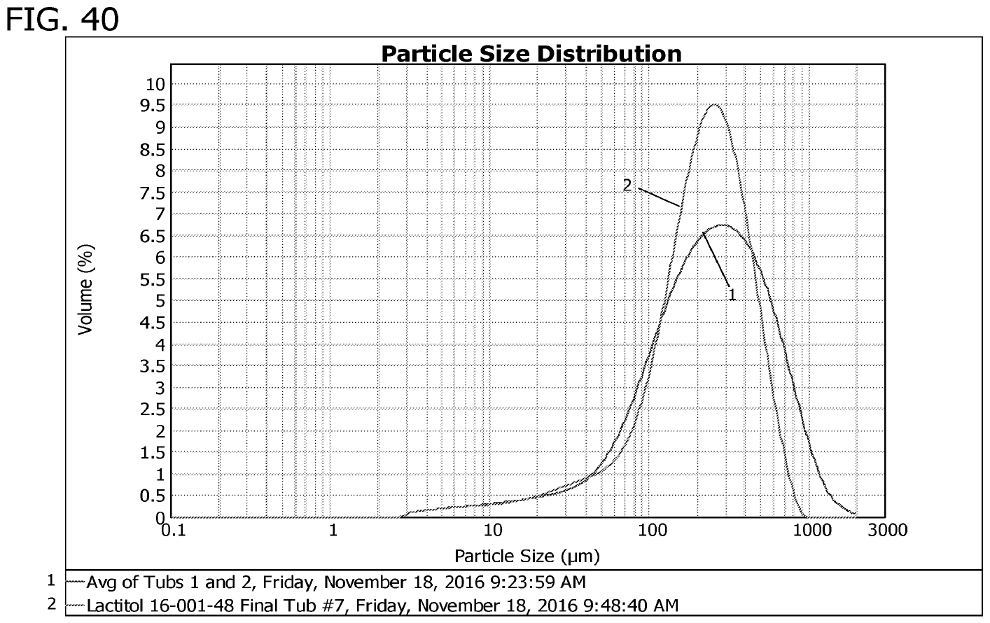

As shown, the wet granulation moisture of Tub #7 was near the middle of the range upper end of the currently proposed wet granulation moisture at 8.1%. This is equivalent to a 3.1% moisture gain, during granulation. While the ROTAP particle size results met the proposed limits for the lactitol granulation, the particle size distribution was shifted slightly to the coarser end of the range as evident by the slight increase amounts retained on the 40 and 60 mesh screens as compared to the granulations produced at target conditions (Tubs 1 & 2) and reduced amounts of finer material on the 200 mesh screen and in the receiving pan. The MALVERN particle size testing depicted in FIG. 40 reveals that the d50 particle size of the material from Tub #7 is approximately the same as observed with Tubs #1 & #2 (target conditions) however the particle size distribution is narrower. As shown in Table 32, the amount of oversize material (plus 14 TBC screen) produced at the higher production rate condition was higher than the granulation produced at target conditions (˜7% versus ˜2%). Further, the tapped density of the finished granulation did not meet the minimum of 0.82 g/mL at the high production rate conditions.

Tubs 8 and 9:

As shown, the wet granulation moisture for both tubs was approximately 2.6% just slightly below the target. The final moisture after the heated drying cycle was the same for both tubs (5.1% KF) and both met the requirement of NMT 5.5% moisture. Based on these results the outlet temperature endpoint range of 50-55° C. is suitable for the heated drying processing step to ensure the final moisture requirement are met.

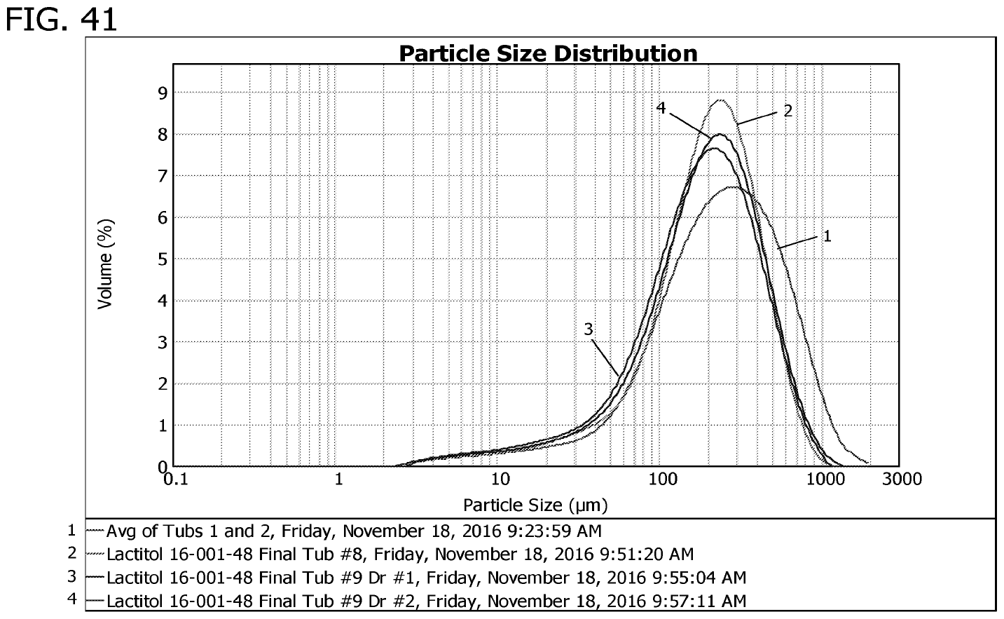

While the ROTAP particle size results met the proposed limits for the lactitol granulation, the particle size distribution was shifted slightly to the finer end of the range as evident by the decrease in the amount retained on the 40 mesh screen and the slight increase of material retained on the 100 mesh screen compared to the previous granulations produced at target conditions (Tubs 1 & 2). The MALVERN particle size testing depicted in FIG. 41 also reflects this shift in particle size. The average d50 particle size for Tubs 8 and 9 was approximately 197 micron compared to an average of approximately 249 microns for the previous granulations produced at target conditions. As shown in Table 32, the amount of oversize material (plus 14 TBC screen) produced from these tubs ranged from approximately 4 to 6%. Further, the tapped density of the finished granulation did not meet the minimum of 0.82 g/mL during execution of this trial.

Tubs 12 to 16:

As can be seen, the wet granulation moistures were all within the proposed range of 7-9.5% with the average moisture gain of 2.7% over the five tubs produced. The average moisture after the final heated drying cycle was 5.0%.

As show above in Table 31, the ROTAP particle size results on the finished product produced during the extended run met the proposed limits. For all tubs, the majority of the material is retained on the 40, 60, and 100 mesh screens with only a small amount material minus 200 mesh. The MALVERN particle size data shows that the d50 results on the finished product is very consistent across all five extended run Tubs. The d50 results ranged from 245-297 microns with an average d50 of 265 microns. This particle size is similar to that achieved during the previously described experimental scale-up run. The data also shows that the tapped density of the finished product produced as part of the extended run meets the minimum density of 0.82 g/mL. The average tapped density across all five tubs was 0.83 g/mL. These passing results are an indication that adjustments made to the ambient fluid bed dryer (inlet temperature controller to zero, steam valves turned off) were effective in maintaining a higher finished product density. The extended run data also shows that the tapped density is not significantly impacted by the sieving/milling operation as indicated by the average tapped density of the “as dried” material being the same as the finished product.

As shown in Table 32, the amount of oversize material (plus 14 TBC screen) produced from each tub during the extended run ranged from 1-8%. The average total net weight of product recovered from each tub (100 kilograms of wet granulation) was approximately 91 kilograms. The finished product from Tub 16 was used to test whether all of the product from a tub could be packaged in one, 41 gallon fiber drum rather than being packaged in 2, 23 gallon drums. The entire tub of finished material from Tub 16 (91.4 kilograms) was successfully packaged in one, double lined 41 gal fiber drum. There was approximately 4-6″ of head space after the drum was filled.

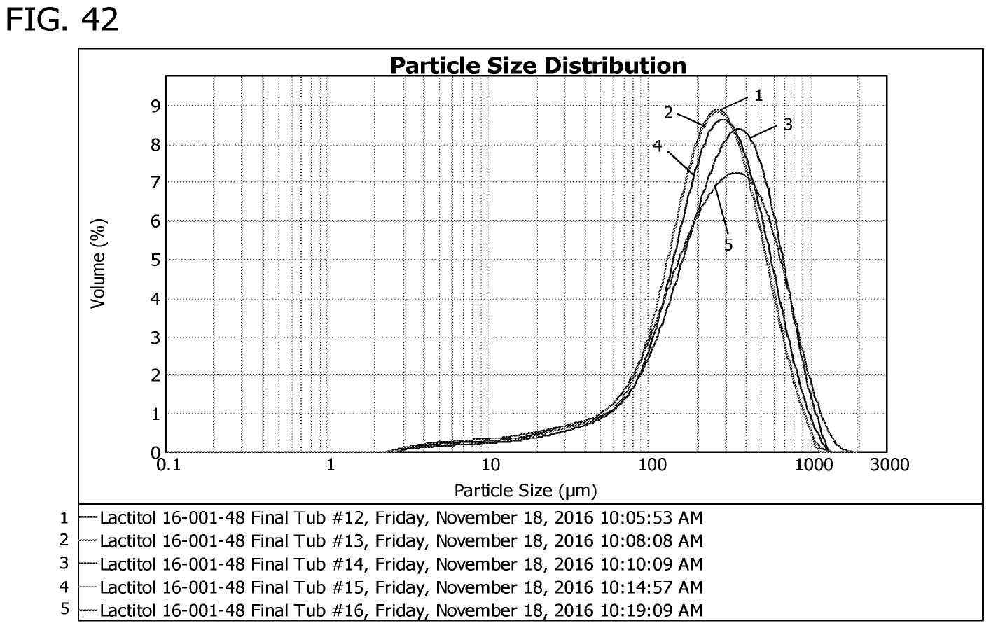

Samples of the lactitol granulation produced during the extended run (Tubs 12-16) were also tested for dissolution rate using the wet sampling attachment for the MALVERN MASTERSIZER 2000. The results are displayed in FIG. 42. The dissolution results of the material produced from Tubs 12-16 were consistent and comparable to the dissolution results obtained with the granulations produced during the previous executed R&D scale-up trial.

Tub 17:

It was hypothesized that addition of the coarser, higher density raw Lactitol would enhance the tapped density of the finished granulation. However, while the resultant granulation had approximately the same ROTAP and MALVERN particle size as the tubs produced during the extended run, the tapped density was not increased. The tapped density was actually slightly less at 0.79 g/mL.

Flowability Testing

Samples of the finished lactitol granulations produced during execution of the commercial optimization protocol were also evaluated for flow using the HANSON Intrinsic Flowability Test. During the test, the smallest hole (in millimeters) that the product will flow through is determined in the FLODEX apparatus. Using this information and the bulk density of each powder (as determined in a 250 mL graduated cylinder) the coefficient of friction or viscosity (K) was calculated. The viscosity K (in poises) is equal to 490 (½ the acceleration of gravity) times the radius of the hole in centimeters times the bulk density of the sample. The result K is then multiplied by 100 to express the viscosity in centipoises. The results for each sample are displayed in Table 33.

TABLE 33
Flowability Results

| | | Flowability | Flowability |
|---|---|---|---|
| | | (Viscosity) | (Viscosity) |
| Sample | Bulk Density | All Data | Tubs w/o dev. |
| Tub | g/mL | cps | cps |
| 1 | 0.667 | 6601 | 6601 |
| 2 | 0.75 | 7350 | 7350 |
| 3 | 0.70 | 6860 | 6860 |
| 4 | 0.69 | 6762 | 6762 |
| 5 | 0.64 | 6272 | 6272 |
| 6 | 0.68 | 6664 | 6664 |
| 7 | 0.63 | 6174 | 6174 |
| 8 | 0.64 | 9272 | 6272 |
| 9 | 0.62 | 6370 | 6370 |
| 10 | 0.68 | 8330 | — |
| 11 | 0.67 | 9849 | — |
| 12 | 0.68 | 6664 | 6664 |
| 13 | 0.68 | 6664 | 6664 |
| 14 | 0.69 | 6762 | 6762 |
| 15 | 0.69 | 6762 | 6762 |
| 16 | 0.70 | 6860 | 6860 |
| 17 | 0.67 | 6566 | 6566 |
| Avg. | 0.68 | 6928.4 | 6640.2 |
| Min. | 0.63 | 6174.0 | 6174.0 |
| Max. | 0.75 | 9849.0 | 7350.0 |
| s | 0.028 | 899.8 | 294.6 |

Overall, the flowability results of the material produced during execution of this commercial optimization protocol are acceptable based on visual observation and the viscosity determination through execution of the Hansen Intrinsic Flowability Test. Overall, the granulation viscosity results are similar across all tubs produced during this study. The various operating conditions used during this study, within the defined ranges specified in the commercial optimization protocol do not significantly impact the finished product viscosity/flowability. The average viscosity of the material produced throughout this optimization trial was approximately 7000 cps. Eliminating deviation tubs 10 and 11 the average viscosity of the material produced was 6600 cps (range: 6100-7400 cps).

Proposed Operating Conditions

Based on the data generated during this Commercial Optimization trial, the following operating ranges for the critical and non-critical operating parameters in Table 34 are recommended for producing lactitol granulation. The listed process parameters that are bold are believed to have the greatest impact on the quality of the finished product and are considered the critical process parameters.

TABLE 34
Proposed Operating Conditions

| Process Step | Parameter | Proposed Operating Range |
|---|---|---|
| Jet Milling of Raw | Jet Mill Feed Injection Pressure | 29-31 psi, Target 30 psi |
| Lactitol | Jet Grinding Pressure | 26-32 psi, Target 28 psi |
| | Power flow rate to mill | Adjust feeder speed to maintain jet |
| | | mill hopper approximately 75% full |
| Binder Make-Up | Water Temperature | 30-40° C., Target 35° C. |
| | Mixing Speed | 280-300 rpm, Target 290 rpm |
| | Mixing Time | 15 minutes (minimum) |
| Granulation | Milled Lactitol Feed Rate | 9.5-13 Kg, Target 11 Kg |
| | Binder Feed Rate | 450-750 g/minute |
| | Impeller Speed | 1000-1450 rpm, Target 1225 rpm |
| | No. of Spray Nozzles/size | Two Spray Nozzles with 0.060″ |
| | | (No. 8) spray tips |
| | Amount of Wet Granulation per | 90-110 Kg per Tub |
| | Drying Tub | |
| | Wet Granulation Moisture | 6.8-9.5% (KF) |
| | Binder Mixing Speed During | 90-110 rpm, Target 100 rpm |
| | Processing | |
| Unheated Drying | Inlet Temperature | Ambient (Set to 0° C., steam off) |
| | Dryer Exhaust Blower Speed | 1200-1700 rpm, Target 1650 rpm |
| Heated Drying | Inlet Temperature | 50-70° C., Target 60° C. |
| | Outlet Temperature | 50-55° C. |
| | Dryer Exhaust Blower Speed | 1200-1700 rpm, Target 1650 rpm |
| Vibratory Sieve | Top Screen size | #14 TBC |
| | Bottom screen size | #24 TBC |
| Jet Milling of Oversize | Jet Mill Feed Injection Pressure | 35-45 psi, Target 40 psi |
| | Jet Grinding Pressure | 35-45 psi, Target 40 psi |

In Process Specifications

Based on the results of this optimization trial, the following updated in-process specifications in Table 35 are desired for the production of qualification lots of Lactitol Granulation.

TABLE 35
Moisture, Particle Size, and Tapped Density Limits

| Parameters | Limits |
|---|---|
| Moisture (KF) Wet Granulation | 6.8-9.5% |
| Moisture (KF) After 2nd Drying Step | NMT 5.5% |
| Particle Size Analysis (% ret) | |
| U.S. Standard #20 | NMT 1% On |
| U.S. Standard #40 | NMT 35% On |
| U.S. Standard #100 | NLT 18% On |
| U.S. Standard 200 mesh | NMT 6% Thru |
| Tapped Density | 0.82-0.90 g/mL |

Product Specifications

Based on the results of this optimization trial, the following updated product specifications in Table 36 are desired for the production of qualification lots of Lactitol Granulation.

TABLE 36
Finished Product Specifications

| Proposed Specification | Acceptance Criteria |
|---|---|
| Appearance | White to off white free flowing, |
| | granular powder |
| Identification | Meets the requirements |
| Assay (dry basis) | 980-1010 mg/g |
| Moisture (% KF) | NMT 5.5% |
| Particle Size Analysis (% ret) | |
| U.S. Standard #20 | NMT 1% On |
| U.S. Standard #40 | NMT 35% On |
| U.S. Standard #100 | NLT 18% On |
| U.S. Standard #200 | NMT 6% Thru |
| Tapped Density | 0.82 to 0.90 g/mL |
| Microbiology | |
| Total Plate Count | ≤1000 cfu/g |
| Coliform | Negative |
| Salmonella | Negative |
| Yeast & Mold | ≤100 cfu/g |

The critical process parameters identified and rationalized as part of the Commercial Optimization Protocol evaluation strategy including the milled Lactitol feed rate, Lactitol binder feed rate, heated drying inlet temperature, and heated drying outlet temperature were confirmed. It was also shown that the granulator impeller speed should not be considered a critical process parameter for this process as this parameter was found to have little to no impact on the particle size, tapped density, or moisture critical quality attributes. Suitable control limit ranges were identified for each critical process parameter as displayed in Table 36. Operating the process with these control limit ranges resulted in the finished Lactitol Granulation meeting all particle size, tapped density, and moisture requirements except for noted deviations.

### Example 4: Qualification Analysis of Scale-Up Lactitol Granulation

The raw materials used for qualification testing are detailed in Tables 37. Corresponding lot numbers are listed in Table 38.

TABLE 37
Raw Materials and Formulation

| Material Description | Item Number | % | Mg/g | Manufacturer | Function |
|---|---|---|---|---|---|
| Lactitol Monohydrate | 9-5064 | 97.37 | 973.7 | SHANDONG LUJIAN | Active |
| | | | | BIOLOGICAL TECHNOLOGY/ | ingredient |
| | | | | DANISCO | |
| Lactitol Monohydrate | 9-5067 | 2.63 | 26.3 | SHANDONG LUJIAN | Binder/Active |
| for Binder Solution | | | | BIOLOGICAL TECHNOLOGY/ | Ingredient |
| | | | | DANISCO | |
| Purified Water, | 8-51 | 1 | 1 | PD INTERNATIONAL | Vehicle for |
| USP/Ph. Eur. | | | | | Binder |
| | | | | | Solution |

TABLE 38
Raw Material Lot Numbers

| Reactant | Item | | | | |
|---|---|---|---|---|---|
| Description | Number | | Lot 201698 | Lot 201699 | Lot 201700 |
| Raw Lactitol | 9-5064 | Reactant Lot | 127495 | 127495 | 127495 |
| | | Number | | | |
| | | Vendors Lot | 2016090220 | 2016090220 | 2016090220 |
| | | Number | | | |

Operating conditions and in-process parameters are listed in Example 3, Tables 34 and 35. Raw material specifications are detailed in Table 39. Final product specifications are listed in Table 40.

TABLE 39
Raw Material Specifications

| Test Description | Test Method | Acceptance Criteria |
|---|---|---|
| Description | Visual | White or almost white, crystalline powder |
| Identification A (IR) | USP/NF | Matches IR spectrum of reference standard |
| Identification B | FCC | The principal spot obtained from the Sample |
| | | solution corresponds in color and RF value to that |
| | | obtained from the Standard solution |
| Assay (dry basis) | USP/NF | 98.0-101.0% |
| Residue on Ignition | USP/NF | NMT 0.1% |
| Heavy Metals | USP/NF | NMT 5 ppm |
| Lead | USP/NF | NMT 1 mg/Kg |
| Nickel | FCC | NMT 1 mg/Kg |
| Related Compounds | USP/NF | The total of all related compounds is NMT 1.5% |
| Other Hydrogenated | FCC | NMT 4.0% |
| Saccharides | | |
| Reducing Sugars A | FCC | The weight of the cuprous oxide residue is NMT |
| | | 30 mg (NMT 0.3%) |
| Reducing Sugars B | USP/NF | NMT 2.0% calculated as dextrose. The sample |
| | | solution shows no more turbidity than that |
| | | produced in the standard solution |
| Water Determination | USP/NF | 4.5%-5.5% |
| pH (1% Aqueous) | FCC | 4.5-7.0 |
| Residual Solvents | (vendor CofA) | Meets the requirements |

TABLE 40
Final Product Specifications

| Test Description | Specification | U/M | Method # |
|---|---|---|---|
| Appearance | White to off white free flowing | | 9-5065 |
| | powder | | |
| Identification - Specific | ⁺13.5 to ⁺15.5 | | 9-5065 |
| Optical Rotation | (anhydrous substance) | | |
| Identification - IR | Compares to Standard | | 9-5065 |
| Absorption | | | |
| Assay | 98.0-101.0 | | 9-5065 |
| Related Substances | Total - NMT 1.5 | % | 9-5065 |
| Reducing Sugars | NMT 0.2 | % | 9-5065 |
| Lead | NMT 0.5 | ppm | 9-5065 |
| Nickel | NMT 1 | ppm | 9-5065 |
| Heavy Metals | NMT 5 | ppm | 9-5065 |
| Sulfated Ash | NMT 0.1 | % | 9-5065 |
| Tapped Density | 0.82-0.90 | g/mL | 9-5065 |
| Moisture (KF) | 4.5-5.5 | % | 9-5065 |
| Particle Size #20 US Std. | NMT 1 | % on | 9-5065 |
| Sieve | | | |
| Particle Size #40 US Std. | NMT 35 | % on | 9-5065 |
| Sieve | | | |
| Particle Size #100 US Std. | NLT 18 | % on | 9-5065 |
| Sieve | | | |
| Particle Size #200 US Std. | NMT 6 | % | 9-5065 |
| Sieve | | thru | |
| Total Microbial Count | NMT 1,000 | cfu/g | USP <61> |
| Total Yeast and Mold Count | NMT 100 | cfu/g | USP <61> |
| E. Coli | Negative | | USP <62> |
| Salmonella Species | Negative | | USP <62> |
| Pseudomonas Aeruginosa | Negative | | USP <62> |
| Appearance of Solution | No more intense than Reference | | EP method for Lactitol |
| | Solution BY7 | | Monohydrate |
| Acidity or Alkalinity | NMT 0.2 mL 0.01M NaOH | | EP method for Lactitol |
| | NMT 0.3 mL 0.01M HC1 | | Monohydrate |

Raw Material Testing

Before the production of the qualification batches began, a sample of the raw crystal Lactitol Monohydrate (Product Code: 9-5064, Lot #127495) was taken and sent out for testing. The testing results and specifications are shown in Table 41.

TABLE 41
Raw Material Testing - Crystal Lactitol Monohydrate

| Test Description | Test Method | Acceptance Criteria | Lot 127495 |
|---|---|---|---|
| Description | Visual | White or almost white, cry stalline powder | Pass |
| Identification A (IR) | USP/NF | Matches IR spectrum of reference standard | Pass |
| Identification B | FCC | The principal spot obtained from | Pass |
| | | the Sample solution corresponds in color | |
| | | and RF value to that obtained from | |
| | | the Standard solution | |
| Assay (dry basis) | USP/NF | 98.0-101.0% | 99.7% Avg |
| | | | (99.5, 99.8, 99.8) |
| Residue on Ignition | USP/NF | NMT 0.1% | 0.0% |
| Heavy Metals | USP/NF | NMT 5 ppm | Pass |
| Lead | USP/NF | NMT 1 mg/Kg | Pass |
| Nickel | FCC | NMT 1 mg/Kg | Pass |
| Related Compounds | USP/NF | The total of all related compounds is | 0.76% |
| | | NMT 1.5% | 0.56% |
| | | | 0.67% |
| Other Hydrogenated | FCC | NMT 4.0% | 0.7% |
| Saccharides | | | 0.6% |
| | | | 0.4% |
| Reducing Sugars A | FCC | The weight of the cuprous oxide residue is | 10 mg |
| | | NMT 30 mg (NMT 0.3%) | (Pass) |
| Reducing Sugars B | USP/NF | NMT 2.0% calculated as dextrose. The | Pass |
| | | sample solution shows no more turbidity | |
| | | than that produced in the standard solution | |
| Water Determination | USP/NF | 4.5%-5.5% | 5.1% |
| pH (1% Aqueous) | FCC | 4.5-7.0 | 5.9 |
| Residual Solvents | (vendor CofA) | Meets the requirements | Pass |

In-Process Testing

Moisture Content (KF)—in-Process (Wet Granulation):

During the weighing of the ingredients for the binder solution and during the set up for granulation, an approximately 100 g sample representing each raw material is taken and placed into an appropriately labeled container. An approximately 0.3 gram sample is dispersed in methanol and test using a Karl Fischer moisture analyzer with HYDRANAL Composite 5 titrant using a 60 sec extraction time. Percent moisture was reported.

For each Qualification lot, at the beginning and middle of each shift samples were pulled using a sample thief from three locations within the tub after granulation but before drying. These samples were tested for moisture as described above. The results are in Table 42.

TABLE 42
Moisture Content

| | |
|---|---|
| | Wet |
| | Granulation |
| Tub# | Moisture |
| Acceptance | NMT 9.5 |
| Criteria | % KF |

Lot#201698

| | |
|---|---|
| 1 | 7.3 |
| 12 | 7.2 |
| 18 | 7.6 |
| 24 | 7.4 |
| 34 | 7.4 |
| 38 | 7.3 |
| 50 | 7.4 |
| Average | 7.37 |
| Stdev | 0.13 |
| % RSD | 1.7 |
| Min | 7.2 |
| Max | 7.6 |

Lot # 201699

| | |
|---|---|
| 1 | 7.6 |
| 8 | 7.6 |
| 19 | 7.5 |
| 30 | 7.1 |
| 37 | 7.5 |
| 46 | 7.2 |
| Average | 7.42 |
| Stdev | 0.21 |
| % RSD | 2.9 |
| Min | 7.1 |
| Max | 7.6 |

Lot # 201700

| | |
|---|---|
| 1 | 7.3 |
| 11 | 7.0 |
| 16 | 7.0 |
| 27 | 6.8 |
| 35 | 7.3 |
| 50 | 7.4 |
| 53 | 7.0 |
| Average | 7.11 |
| Stdev | 0.22 |
| % RSD | 3.1 |
| Min | 6.8 |
| Max | 7.4 |

All results for the Wet Granulation moisture were within acceptance criteria with a % RSD of <3.2%, indicating excellent control over the granulation process.

Moisture content was also tested using the same procedure after the second drying step. Results are listed in Table 43 and summarized in Table 44.

TABLE 43
In-Process Moisture

| | In-Process Moisture (%) | | |
|---|---|---|---|
| | Lot # | | |
| | 201698 | 201699 | 201700 |
| Tub | NMT 5.5% | NMT 5.5% | NMT 5.5% |
| 1 | 4.3 | 4.6 | 4.9 |
| 2 | 4.4 | 4.8 | 5.0 |
| 3 | 4.7 | 5.0 | 5.0 |
| 4 | 4.3 | 4.9 | 4.8 |
| 5 | 4.5 | 4.9 | 4.9 |
| 6 | 4.8 | 5.1 | 4.9 |
| 7 | 4.7 | 4.9 | 5.0 |
| 8 | 4.8 | 5.1 | 5.0 |
| 9 | 4.9 | 4.9 | 4.9 |
| 10 | 4.8 | 4.9 | 4.9 |
| 11 | 4.9 | 4.9 | 4.9 |
| 12 | 4.8 | 4.8 | 5.0 |
| 13 | 4.7 | 4.9 | 4.9 |
| 14 | 5.0 | 4.8 | 5.0 |
| 15 | 4.8 | 4.9 | 4.9 |
| 16 | 5.0 | 4.9 | 4.9 |
| 17 | 4.9 | 4.9 | 5.0 |
| 18 | 4.8 | 5.0 | 5.0 |
| 19 | 5.0 | 4.9 | 5.0 |
| 20 | 4.8 | 4.9 | 5.0 |
| 21 | 4.6 | 4.8 | 5.0 |
| 22 | 4.8 | 4.9 | 5.0 |
| 23 | 4.6 | 4.9 | 5.0 |
| 24 | 4.5 | 4.8 | 4.9 |
| 25 | 4.6 | 4.9 | 4.8 |
| 26 | 4.9 | 5.0 | 4.9 |
| 27 | 4.4 | 4.9 | 4.8 |
| 28 | 4.3 | 4.9 | 4.9 |
| 29 | 4.7 | 4.4 | 4.6 |
| 30 | 4.9 | 4.7 | 4.9 |
| 31 | 3.8 | 4.9 | 4.8 |
| 32 | 4.8 | 5.0 | 4.9 |
| 33 | 4.2 | 4.9 | 4.9 |
| 34 | 3.5 | 5.0 | 4.9 |
| 35 | 3.9 | 4.8 | 4.9 |
| 36 | 4.5 | 4.9 | 4.9 |
| 37 | 4.8 | 4.9 | 5.0 |
| 38 | 4.8 | 5.0 | 5.0 |
| 39 | 4.5 | 4.9 | 4.9 |
| 40 | 4.7 | 4.9 | 5.0 |
| 41 | 4.5 | 4.9 | 5.0 |
| 42 | 4.2 | 4.7 | 5.0 |
| 43 | 4.8 | 4.8 | 5.0 |
| 44 | 4.9 | 5.0 | 5.0 |
| 45 | 5.5 | 4.9 | 5.0 |
| 46 | 4.9 | 4.8 | 4.9 |
| 47 | 4.6 | 5.0 | 4.9 |
| 48 | 4.7 | 5.1 | 5.1 |
| 49 | 4.5 | 4.5 | 5.0 |
| 50 | 4.7 | 5.0 | 5.1 |
| 51 | 4.5 | 5.0 | 4.9 |
| 52 | 5.4 | 4.9 | 5.0 |
| 53 | — | — | 4.9 |
| 54 | — | — | 5.0 |
| 55 | — | — | 5.0 |
| 56 | — | — | 4.9 |

TABLE 44
Summary of In-Process Moisture Results

| | In-Process Moisture (%) | | |
|---|---|---|---|
| | Lot # | | |
| | 201698 | 201699 | 201700 |
| Average | 4.7 | 4.9 | 4.9 |
| Stdev | 0.3 | 0.1 | 0.1 |
| Min | 3.5 | 4.4 | 4.6 |
| Max | 5.5 | 5.1 | 5.1 |
| Final Composite Result | 4.8 | 5.0 | 5.0 |

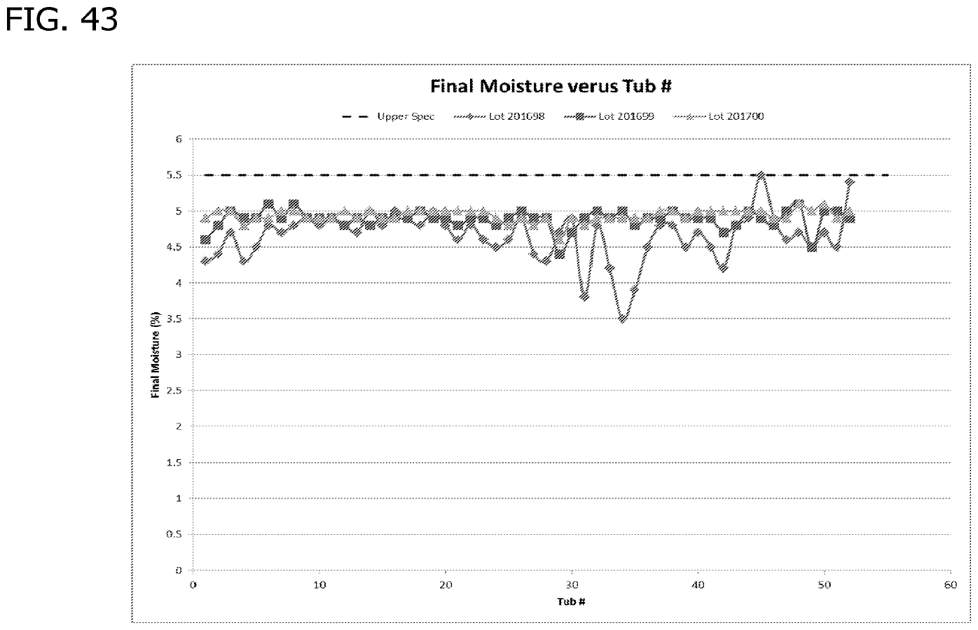

As shown in FIG. 43, all results for the final product moisture were within the original acceptance criteria of NMT 5.5% with standard deviations of <0.4%. Lot 210698 (the first lot produced) had higher variation than either of the other two lots. The low moisture results for lot 201698 can be attributed to the large drop in outside temperature and humidity experienced on the day of production of this lot. While none of these moisture results was out-of-specification (the in-process spec was NMT 5.5%), operators were instructed to better target moisture of 5.0% for the next two lots.

Particle Size: During the granulation process, an approximately 300 g sample is taken from material of the finished product drum starting with the first drum and every sixth drum thereafter, including the last drum (i.e. drum #1, 7, 13, 19 etc.). This sample is immediately tested for particle size by ROTAP on a stack of the following sieves: U.S. Std #12, U.S. Std #20, U.S. Std #40, U.S. Std #60, U.S. Std #100, U.S. Std #200, and a receiving pan for 5 minutes. Each fraction was weighed and percent retained was calculated. The samples exhibited the results in Tables 45 to 47.

TABLE 45
Particle Size Distribution for Lot #201698

| | | Particle Size Distribution (% Retained) | | | | | | |
|---|---|---|---|---|---|---|---|---|
| Tub | Drum | Report | NMT 1% | NMT 35% | Report | NLT 18% | Report | NMT 6% |
| Acceptance Criteria | | 12 | 20 | 40 | 60 | 100 | 200 | Pan |
| 1 | 1 | 0 | 0 | 15 | 30 | 31 | 22 | 2 |
| 4 | 7 | 0 | 0 | 14 | 27 | 38 | 21 | 1 |
| 7 | 13 | 0 | 0 | 8 | 27 | 42 | 23 | 0 |
| 11 | 19 | 0 | 0 | 11 | 33 | 38 | 18 | 0 |
| 14 | 25 | 0 | 0 | 17 | 40 | 33 | 9 | 1 |
| 17 | 31 | 0 | 0 | 17 | 42 | 30 | 10 | 1 |
| 21 | 37 | 0 | 0 | 14 | 34 | 36 | 16 | 0 |
| 24 | 43 | 0 | 0 | 14 | 38 | 33 | 14 | 1 |
| 28 | 49 | 0 | 0 | 15 | 35 | 32 | 18 | 1 |
| 30 | 55 | 0 | 0 | 14 | 33 | 32 | 20 | 1 |
| 34 | 61 | 0 | 0 | 14 | 35 | 32 | 19 | 1 |
| 37 | 67 | 0 | 0 | 5 | 48 | 34 | 13 | 1 |
| 40 | 73 | 0 | 0 | 20 | 41 | 28 | 11 | 0 |
| 44 | 79 | 0 | 0 | 27 | 39 | 22 | 9 | 3 |
| 48 | 85 | 0 | 0 | 19 | 45 | 28 | 9 | 0 |
| 51 | 91 | 0 | 0 | 16 | 35 | 32 | 16 | 1 |
| 52 | 94 | 0 | 0 | 16 | 36 | 32 | 13 | 2 |

| | | | | | | | |
|---|---|---|---|---|---|---|---|
| Average | 0.0 | 0.0 | 15.1 | 36.4 | 32.5 | 15.4 | 0.9 |
| Stdev | 0.0 | 0.0 | 4.8 | 5.8 | 4.5 | 4.8 | 0.8 |
| Min. | 0.0 | 0.0 | 5.0 | 27.0 | 22.0 | 9.0 | 0.0 |
| Max. | 0.0 | 0.0 | 27.0 | 48.0 | 42.0 | 23.0 | 3.0 |

TABLE 46
Particle Size Distribution for Lot # 201699

| | | Particle Size Distribution (% Retained) | | | | | | |
|---|---|---|---|---|---|---|---|---|
| Tub | Drum | Report | NMT 1% | NMT 35% | Report | NLT 18% | Report | NMT 6% |
| Acceptance Criteria | | 12 | 20 | 40 | 60 | 100 | 200 | Pan |
| 1 | 1 | 0 | 0 | 23 | 46 | 24 | 7 | 1 |
| 5 | 7 | 0 | 0 | 13 | 47 | 33 | 7 | 0 |
| 9 | 13 | 0 | 0 | 22 | 45 | 25 | 7 | 1 |
| 12 | 19 | 0 | 0 | 34 | 37 | 20 | 7 | 1 |
| 15 | 25 | 0 | 0 | 14 | 40 | 32 | 14 | 0 |
| 19 | 31 | 0 | 0 | 17 | 38 | 33 | 11 | 1 |
| 21 | 37 | 0 | 0 | 22 | 39 | 29 | 10 | 1 |
| 25 | 43 | 0 | 0 | 29 | 35 | 26 | 10 | 1 |
| 27 | 49 | 0 | 0 | 23 | 37 | 29 | 11 | 1 |
| 31 | 55 | 0 | 0 | 17 | 29 | 21 | 32 | 1 |
| 34 | 61 | 0 | 0 | 23 | 40 | 29 | 9 | 0 |
| 37 | 67 | 0 | 0 | 19 | 46 | 28 | 7 | 0 |
| 40 | 73 | 0 | 0 | 34 | 37 | 21 | 8 | 0 |
| 44 | 79 | 0 | 0 | 20 | 36 | 30 | 13 | 0 |
| 48 | 85 | 0 | 0 | 16 | 28 | 35 | 21 | 0 |
| 51 | 91 | 0 | 0 | 17 | 47 | 28 | 8 | 0 |
| 52 | 94 | 0 | 0 | 14 | 37 | 39 | 11 | 0 |

| | | | | | | | |
|---|---|---|---|---|---|---|---|
| Average | 0.0 | 0.0 | 21.0 | 39.1 | 28.4 | 11.4 | 0.5 |
| Stdev | 0.0 | 0.0 | 6.4 | 5.7 | 5.2 | 6.4 | 0.5 |
| Min. | 0.0 | 0.0 | 13.0 | 28.0 | 20.0 | 7.0 | 0.0 |
| Max. | 0.0 | 0.0 | 34.0 | 47.0 | 39.0 | 32.0 | 1.0 |

TABLE 47
Particle Size Distribution for Lot # 201700

| | | Particle Size Distribution (% Retained) | | | | | | |
|---|---|---|---|---|---|---|---|---|
| Tub | Drum | Report | NMT 1% | NMT 35% | Report | NLT 18% | Report | NMT 6% |
| Acceptance Criteria | | 12 | 20 | 40 | 60 | 100 | 200 | Pan |
| 1 | 1 | 0 | 0 | 17 | 43 | 32 | 9 | 0 |
| 5 | 7 | 0 | 0 | 19 | 38 | 32 | 11 | 0 |
| 9 | 13 | 0 | 0 | 31 | 44 | 20 | 5 | 1 |
| 12 | 19 | 0 | 0 | 31 | 44 | 20 | 5 | 1 |
| 14 | 25 | 0 | 0 | 19 | 47 | 26 | 7 | 1 |
| 16 | 31 | 0 | 0 | 20 | 40 | 29 | 10 | 1 |
| 20 | 37 | 0 | 0 | 31 | 34 | 23 | 11 | 2 |
| 23 | 43 | 0 | 0 | 29 | 40 | 22 | 7 | 1 |
| 26 | 49 | 0 | 0 | 15 | 37 | 32 | 15 | 1 |
| 30 | 55 | 0 | 0 | 20 | 39 | 32 | 9 | 1 |
| 33 | 61 | 0 | 0 | 16 | 36 | 36 | 12 | 1 |
| 36 | 67 | 0 | 0 | 30 | 42 | 21 | 7 | 1 |
| 39 | 73 | 0 | 0 | 20 | 36 | 31 | 12 | 1 |
| 43 | 79 | 0 | 0 | 30 | 33 | 25 | 11 | 1 |
| 46 | 85 | 0 | 0 | 21 | 45 | 26 | 7 | 1 |
| 49 | 91 | 0 | 0 | 23 | 43 | 25 | 7 | 1 |
| 52 | 97 | 0 | 0 | 18 | 43 | 31 | 8 | 1 |
| 55 | 103 | 0 | 0 | 26 | 43 | 25 | 5 | 1 |
| 56 | 105 | 0 | 0 | 13 | 33 | 31 | 20 | 3 |

| | | | | | | | |
|---|---|---|---|---|---|---|---|
| Average | 0.0 | 0.0 | 22.6 | 40.0 | 27.3 | 9.4 | 1.1 |
| Stdev | 0.0 | 0.0 | 6.1 | 4.3 | 4.8 | 3.8 | 0.6 |
| Min. | 0.0 | 0.0 | 1.3 | 33.0 | 20.0 | 5.0 | 0.0 |
| Max. | 0.0 | 0.0 | 31.0 | 47.0 | 36.0 | 20.0 | 3.0 |

All results for the Particle Size Distribution were within acceptance criteria with standard deviations of ≤5.8%, ≤6.4%, and ≤6.1%, respectively, indicating good control over the granulation and drying process.

Density (Bulk and Tap Density):

Tare a 250 mL graduated glass cylinder. Fill with 100 grams of sample. Reweigh to determine weight. Record Initial Volume. The bulk density is the weight in grams divided by the volume (mL). Tap 1000 times by the automated tapping device. Read final volume. The tapped density is the weight in grams divided by the tapped volume (mL).

TABLE 48
Bulk Density and Tapped Density

| | | Bulk | Tapped |
|---|---|---|---|
| | | Density | Density |
| | | Acceptance Criteria | |
| | | — | 0.82-0.90 |
| Tub | Drum | g/mL | g/mL |

Lot # 201698

| | | | |
|---|---|---|---|
| 1 | 1 | 0.746 | 0.877 |
| 4 | 7 | 0.725 | 0.833 |
| 7 | 13 | 0.735 | 0.833 |
| 11 | 19 | 0.746 | 0.847 |
| 14 | 25 | 0.735 | 0.833 |
| 17 | 31 | 0.714 | 0.833 |
| 21 | 37 | 0.714 | 0.893 |
| 24 | 43 | 0.714 | 0.862 |
| 28 | 49 | 0.735 | 0.833 |
| 30 | 55 | 0.725 | 0.847 |
| 34 | 61 | 0.725 | 0.847 |
| 37 | 67 | 0.725 | 0.862 |
| 40 | 73 | 0.758 | 0.877 |
| 44 | 79 | 0.704 | 0.847 |
| 48 | 85 | 0.758 | 0.877 |
| 51 | 91 | 0.746 | 0.862 |
| 52 | 94 | 0.735 | 0.833 |

| | | |
|---|---|---|
| Average | 0.732 | 0.853 |
| Stdev | 0.016 | 0.019 |
| % RSD | 2.1 | 2.3 |
| Min. | 0.704 | 0.833 |
| Max. | 0.758 | 0.893 |

Lot # 201699

| | | | |
|---|---|---|---|
| 1 | 1 | 0.746 | 0.847 |
| 5 | 7 | 0.735 | 0.833 |
| 9 | 13 | 0.735 | 0.847 |
| 12 | 19 | 0.746 | 0.862 |
| 15 | 25 | 0.725 | 0.847 |
| 19 | 31 | 0.725 | 0.847 |
| 21 | 37 | 0.735 | 0.847 |
| 25 | 43 | 0.735 | 0.877 |
| 27 | 49 | 0.735 | 0.847 |
| 31 | 55 | 0.735 | 0.862 |
| 34 | 61 | 0.758 | 0.862 |
| 37 | 67 | 0.735 | 0.833 |
| 40 | 73 | 0.758 | 0.893 |
| 44 | 79 | 0.746 | 0.862 |
| 48 | 85 | 0.758 | 0.877 |
| 51 | 91 | 0.735 | 0.847 |
| 52 | 94 | 0.746 | 0.847 |

| | | |
|---|---|---|
| Average | 0.741 | 0.855 |
| Stdev | 0.010 | 0.016 |
| % RSD | 1.4 | 1.9 |
| Min. | 0.725 | 0.833 |
| Max. | 0.7558 | 0.893 |

Lot # 201700

| | | | |
|---|---|---|---|
| 1 | 1 | 0.725 | 0.833 |
| 5 | 7 | 0.746 | 0.862 |
| 9 | 13 | 0.758 | 0.847 |
| 12 | 19 | 0.758 | 0.847 |
| 14 | 25 | 0.714 | 0.847 |
| 16 | 31 | 0.725 | 0.847 |
| 20 | 37 | 0.735 | 0.893 |
| 23 | 43 | 0.735 | 0.847 |
| 26 | 49 | 0.735 | 0.862 |
| 30 | 55 | 0.725 | 0.862 |
| 33 | 61 | 0.725 | 0.877 |
| 36 | 67 | 0.769 | 0.877 |
| 39 | 73 | 0.769 | 0.877 |
| 43 | 79 | 0.769 | 0.893 |
| 46 | 85 | 0.735 | 0.847 |
| 49 | 91 | 0.746 | 0.862 |
| 52 | 97 | 0.735 | 0.847 |
| 55 | 103 | 0.746 | 0.862 |
| 56 | 105 | 0.735 | 0.847 |

| | | |
|---|---|---|
| Average | 0.741 | 0.860 |
| Stdev | 0.017 | 0.017 |
| % RSD | 2.2 | 1.9 |
| Min. | 0.714 | 0.833 |
| Max. | 0.769 | 0.893 |

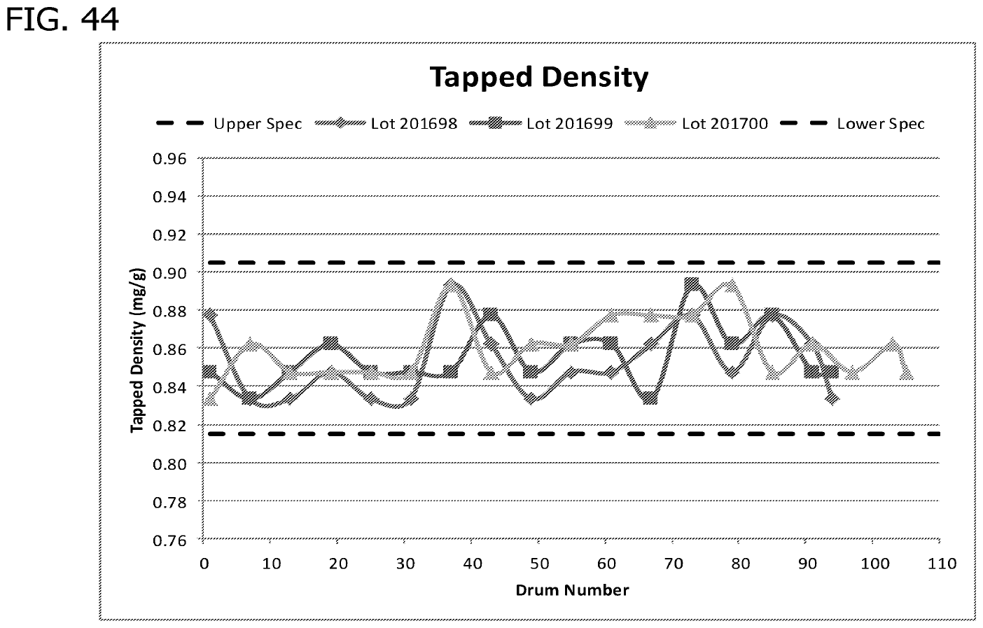

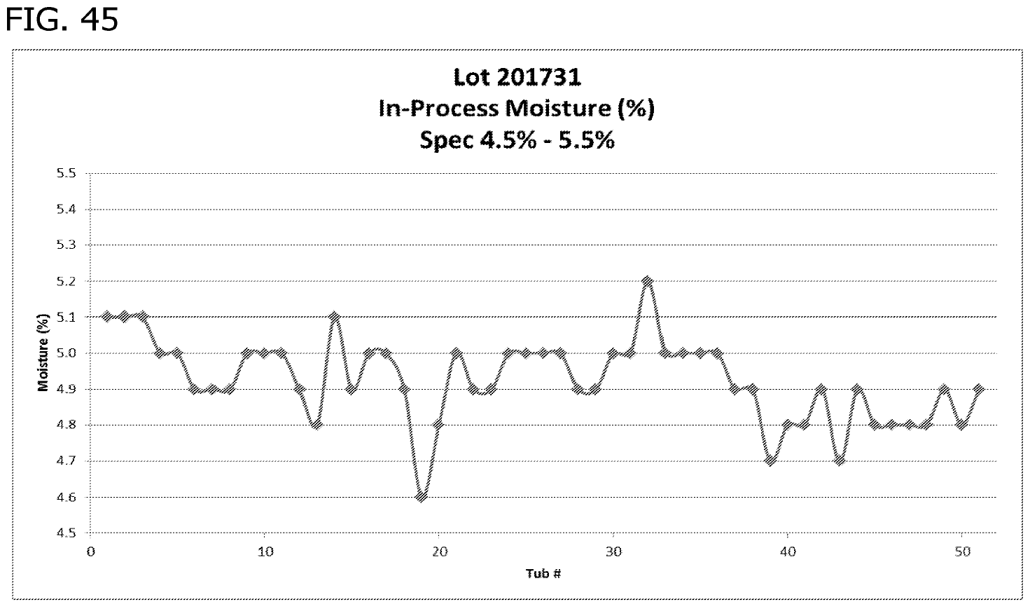

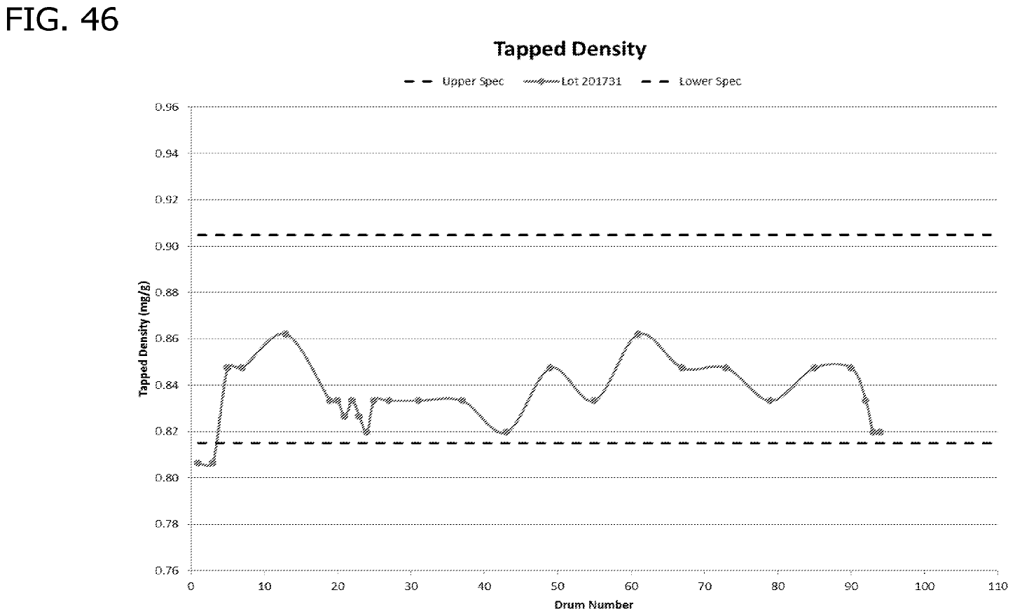

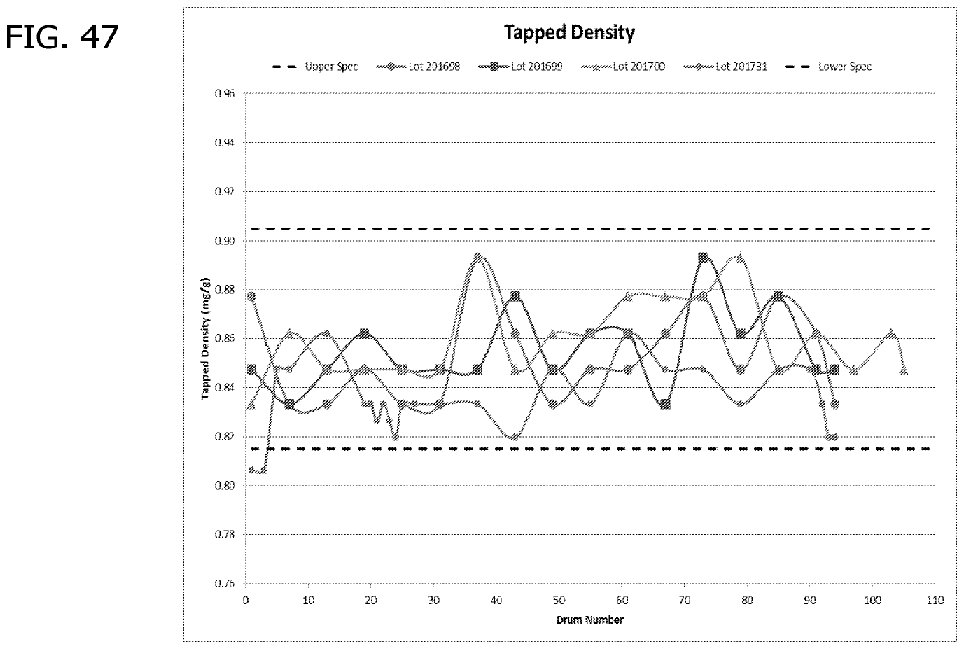

As shown in Table 48 and FIG. 44, all results for the Tapped Density were within acceptance criteria with a % RSD≤2.3%, indicating excellent control over the granulation process and drying process.

Finished Product Testing

During the granulation process, an approximately 60 g sample was taken from material of the finished product drum starting with the first drum and every third drum thereafter, including the last drum (i.e. drum #1, 7, 13, 19 etc.) and composited as a QA sample. These samples are used for finished product testing. To obtain samples for micro testing, during the granulation process, an approximately 4 oz. sample is taken from material of the finished product drum at the beginning, middle, and end of the lot and placed into a sterile, labeled sample container. These samples are used for micro testing.

Appearance of Solution:

A sample is tested according the degree of coloration with the sample being no more intensely colored than reference solution BY7.

Acidity or Alkalinity:

To 10 mL of solution S add 10 mL of carbon dioxide-free water R. To 10 mL of this solution add 0.05 mL of phenolphthalein solution R. Not more than 0.2 mL of 0.01 M sodium hydroxide is required to change the color of the indicator to pink. To a further 10 mL of the solution add 0.05 mL of methyl red solution R. Not more than 0.3 mL of 0.01 M hydrochloric acid is required to change the color of the indicator to red.

For each Qualification lot, samples were pulled using a sample thief from at least three locations within every sixth (6th) drum and a composite sample was sent for final testing. The testing results and specifications are shown in Table 49.

TABLE 49
Finished Product Testing

| Proposed Specification | Acceptance Criteria | Lot 201698 | Lot 201699 | Lot 201700 |
|---|---|---|---|---|
| Appearance | White to off white free | Passes | Passes | Passes |
| | flowing powder | | | |
| Identification (IR) | Compares to Standard | Passes | Passes | Passes |
| Assay (dry basis) | 98.0-101.0% | 100.2% | 100.4% | 99.0% |
| Moisture (% KF) | 4.5%-5.5% | 4.8% | 5.0% | 5.0% |
| Particle Size Analysis (% ret) | | | | |
| U.S. Standard #20 | NMT 1% On | 0% | 0% | 0% |
| U.S. Standard #40 | NMT 35% On | 15% | 20% | 19% |
| U.S. Standard #100 | NLT 18% On | 61% | 68% | 65% |
| U.S. Standard #200 | NMT 6% Thru | 5% | 2% | 3% |
| Tapped Density | 0.82 to 0.90 g/mL | 0.88 | 0.85 | 0.85 |
| Microbiology | | | | |
| Total Plate Count | ≤1000 cfu/g | <10 | <10 | <10 |
| Yeast & Mold | ≤100 cfu/g | <10 | <10 | <10 |
| E. Coli | Negative | Passes | Passes | Passes |
| Salmonella | Negative | Passes | Passes | Passes |
| Pseudomonas A. | Negative | Passes | Passes | Passes |
| Optical Rotation | +13.5° to +15.5° | +14.8 | +14.8 | +14.7 |
| Related Substances | Total − NMT 1.5% | 0.57% | 0.67% | 1.25% |
| Reducing Sugars | NMT 0.2% | Pass | Pass | Pass |
| Lead | NMT 0.5 ppm | Pass | Pass | Pass |
| Nickel | NMT 1 ppm | Pass | Pass | Pass |
| Heavy Metals | NMT 5 ppm | Pass | Pass | Pass |
| Sulfated Ash | NMT 0.1% | 0.0% | 0.0% | 0.0% |
| Appearance of Solution | No more intense than | Pass | Pass | Pass |
| | Reference Solution BY7 | | | |
| Acidity or Alkalinity | NMT 0.2 mL 0.01M NaOH | Pass | Pass | Pass |
| | NMT 0.3 mL 0.01M HCl | | | |

### Example 5

This example provides an addendum to results provided in Example 4 and adds testing for a Lot 201731 manufactured in April 2017. Due to issues with the in-process moisture for Lot 201698, a fourth lot (Lot 201731) was manufactured in April 2017 with a new lower moisture specifications of NLT 4.5%. Results from Lot 201731 are included in this example.

The following table provides the raw material information.

TABLE 50

| Reactant | Item | | | | | |
|---|---|---|---|---|---|---|
| Description | Number | | Lot 201698 | Lot 201699 | Lot 201700 | Lot 201731 |
| Raw | 9-5064 | Reactant | 127495 | 127495 | 127495 | 127585 |
| Lactitol | | Lot | | | | |
| | | Number | | | | |
| | | Vendors | 2016090220 | 2016090220 | 201609020 | 2017022520 |
| | | Lot | | | | |
| | | Number | | | | |

The following table provides operating ranges for process parameters.

TABLE 51

| Process Step | Parameter | Operating Range |
|---|---|---|
| Jet Milling of | Jet Mill Feed Injection Pressure | 29-31 psi, Target 30 psi |
| Raw Lactitol | Jet Grinding Pressure | 26-32 psi, Target 28 psi |
| | Powder flow rate to mill | Adjust feeder speed to maintain jet mill |
| | | hopper approximately 75% full |
| Binder Make-Up | Water Temperature | 30-40° C., Target 35° C. |
| | Mixing Speed | 280-300 rpm, Target 290 rpm |
| | Mixing Time | 15 minutes (minimum) |
| Granulation | Milled Lactitol Feed Rate | 9.5-13 Kg, Target 11 Kg |
| (Turbulizer) | Binder Feed Rate | 450-750 g/minute |
| | Turbulizer Impeller Speed | 1000-1450 rpm, Target 1225 rpm |
| | No. of Spray Nozzles/size | Two Spray Nozzles with 0.060″ (No. 8) |
| | | spray tips |
| | Amount of Wet Granulation per | 90-110 Kg per Tub |
| | Drying Tub | |
| | Wet Granulation Moisture | 6.8-9.5% (KF) |
| | Binder Mixing Speed During | 90-110 rpm, Target 100 rpm |
| | Procession | |
| Unheated Drying | Inlet Temperature | Ambient (Set to 12° C.) |
| | Dryer Exhaust Blower Speed | 1200-1700 rpm, Target 1650 rpm |
| Heated Drying | Inlet Temperature | 50-70° C., Target 60° C. |
| | Outlet Temperature | 45-55° C. (Changed from 50-55° C.) |
| | Dryer Exhaust Blower Speed | 1200-1700 rpm, Target 1650 rpm |
| Vibratory Sieve | Top Screen size | #14 TBC |
| | Bottom screen size | #24 TBC |
| Jet Milling of | Jet Mill Feed Injection Pressure | 35-45 psi, Target 40 psi |
| Oversize | Jet Grinding Pressure | 35-45 psi, Target 40 psi |

The following table provides in-process testing parameters.

TABLE 52

| Parameters | Limits |
|---|---|
| Moisture (KF) Wet Granulation | NMT 6.8-9.5% |
| Moisture (KF) After 2nd Drying Step | 4.5%-5.5% |
| | (Changed from NMT 5.5%) |
| Particle Size Analysis (% ret) | |
| U.S. Standard #20 | NMT 1% On |
| U.S. Standard #40 | NMT 35% On |
| U.S. Standard #100 | NLT 18% On |
| U.S. Standard #200 | NMT 6% Thru |
| Tapped Density | 0.82-0.90 g/mL |

Before the production of the qualification batches began, a sample of the raw crystal Lactitol Monohydrate (Product Code: 9-5064, Lot #127495) was taken and sent out for testing. Additionally, before the production of the fourth (4^{th}) batch began, a sample of the raw crystal Lactitol Monohydrate (Product Code: 9-5064, Lot #127585) was taken and sent out for testing. The testing results for both lots and specifications are shown in the table below.

TABLE 53

| Test Description | Test Method | Acceptance Criteria | Lot 127495 | Lot 127585 |
|---|---|---|---|---|
| Description | Visual | White or almost white, crystalline powder | Pass | Pass |
| Identification A (IR) | USP/NF | Matches IR spectrum of reference standard | Pass | Pass |
| Identification B | FCC | The principal spot obtained from the Sample | Pass | Pass |
| | | solution corresponds in color and RF value to | | |
| | | that obtained from the Standard solution | | |
| Assay (dry basis) | USP/NF | 98.0-101.0% | 99.7% Avg | 98.9% Avg |
| | | | (99.5, 99.8, 99.8) | |
| Residue on Ignition | USP/NF | NMT 0.1% | 0.0% | 0.0% |
| Heavy Metals | USP/NF | NMT 5 ppm | Pass | Pass |
| Lead | USP/NF | NMT 1 mg/Kg | Pass | Pass |
| Nickel | FCC | NMT 1 mg/Kg | Pass | Pass |
| Related Compounds | USP/NF | The total of all related compounds is | 0.76% | 0.3% |
| | | NMT 1.5% | 0.56% | |
| | | | 0.67% | |
| Other Hydrogenated | FCC | NMT 4.0% | 0.7% | 0.7% |
| Saccharides | | | 0.6% | |
| | | | 0.4% | |
| Reducing Sugars A | FCC | The weight of the cuprous oxide | 10 mg | N/A |
| | | residue is NMT 30 mg (NMT 0.3%) | (Pass) | |
| Reducing Sugars B | USP/NF | NMT 2.0% calculated as dextrose. | Pass | Pass |
| | | The sample solution shows no more | | |
| | | turbidity than that produced in the | | |
| | | standard solution | | |
| Water Determination | USP/NF | 4.5%-5.5% | 5.1% | 5.0% |
| pH (1% Aqueous) | FCC | 4.5-7.0 | 5.9 | 4.9 |
| Residual Solvents | (vendor | Meets the requirements | Pass | Pass |
| | CofA) | | | |

The in-process wet granulation results for Lot 201731 are shown below.

TABLE 54
Lot # 201731

| | Wet |
|---|---|
| | Granulation |
| Tub # | Moisture |
| Acceptance | NMT 9.5 |
| Criteria | % KF |
| 1 | 9.4 |
| 2 | 9.0 |
| 3 | 8.6 |
| 4 | 8.4 |
| 5 | 7.5 |
| 14 | 7.8 |
| 15 | 7.5 |
| 19 | 7.7 |
| 20 | 8.0 |
| 23 | 7.7 |
| 29 | 7.4 |
| 38 | 8.0 |
| 39 | 7.5 |
| Average | 8.04 |
| Stdev | 0.63 |
| % RSD | 7.9 |
| Min | 7.4 |
| Max | 9.4 |

All results for the Wet Granulation moisture were within acceptance criteria with a % RSD of <8%, indicating good control over the granulation process. While all results were within acceptance criteria, the first 4 tubs were at the higher end of the desired range. The binder solution flow rate was therefore reduced to bring the Wet Granulation Moisture to the target of ˜7.5%. From Tub #5 onward, the results ranged between 7.4%-8.0% with a % RSD of <3.0%, indicating excellent control over the granulation process.

The final in-process moisture results for Lot 201731 are shown below.

TABLE 55
Moisture (%)

| | Lot # |
|---|---|
| | 201731 |
| Tub | 4.5%-5.5% |
| 1 | 5.1 |
| 2 | 5.1 |
| 3 | 5.1 |
| 4 | 5.0 |
| 5 | 5.0 |
| 6 | 4.9 |
| 7 | 4.9 |
| 8 | 4.9 |
| 9 | 5.0 |
| 10 | 5.0 |
| 11 | 5.0 |
| 12 | 4.9 |
| 13 | 4.8 |
| 14 | 5.1 |
| 15 | 4.9 |
| 16 | 5.0 |
| 17 | 5.0 |
| 18 | 4.9 |
| 19 | 4.6 |
| 20 | 4.8 |
| 21 | 5.0 |
| 22 | 4.9 |
| 23 | 4.9 |
| 24 | 5.0 |
| 25 | 5.0 |
| 26 | 5.0 |
| 27 | 5.0 |
| 28 | 4.9 |
| 29 | 4.9 |
| 30 | 5.0 |
| 31 | 5.0 |
| 32 | 5.2 |
| 33 | 5.0 |
| 34 | 5.0 |
| 35 | 5.0 |
| 36 | 5.0 |
| 37 | 4.9 |
| 38 | 4.9 |
| 39 | 4.7 |
| 40 | 4.8 |
| 41 | 4.8 |
| 42 | 4.9 |
| 43 | 4.7 |
| 44 | 4.9 |
| 45 | 4.8 |
| 46 | 4.8 |
| 47 | 4.8 |
| 48 | 4.8 |
| 49 | 4.9 |
| 50 | 4.8 |
| 51 | 4.9 |
| 52 | — |
| 53 | — |
| 54 | — |
| 55 | — |
| 56 | — |

TABLE 56

| | In-Process Moisture (%) | | | |
|---|---|---|---|---|
| | Lot # | | | |
| | 201698 | 201699 | 201700 | 201731 |
| Average | 4.7 | 4.9 | 4.9 | 4.9 |
| Stdev | 0.3 | 0.1 | 0.1 | 0.1 |
| Min | 3.5 | 4.4 | 4.6 | 4.6 |
| Max | 5.5 | 5.1 | 5.1 | 5.2 |

Lot 201731 had moisture variations of <0.2%, indicating excellent control over the drying process.

The in-process particle size distribution results for Lot 201731 are shown below.

All results for the Particle Size Distribution were within acceptance criteria (except tub #15) with standard deviations of ≤7.4% indicating good control over the granulation and drying process. Tub #15 (drums 25 and 26) failed with 10% in the pan, these two drums where rejected. Tub #15 was a start-up tub on the second day of manufacturing when a leak occurred in the bender solution line. This resulted in slightly higher fines out of the granulator.

The in-process bulk density and tapped density for Lot 201731 are shown below.

Tubs 1-3 (Drums 1-4) had low Tap Density and were therefore separated from the lot. These drums were later re-sieved as drums 92-94. All results past tub 3 (Drum 4) for the Tapped Density were within acceptance criteria with a % RSD≤1.5%, indicating excellent control over the granulation process and drying process. A graph of the Tapped Density for each of the three exhibit batches plus the fourth (4th) batch is shown below.

Following are the results of finished product testing for the four lots manufactured.

TABLE 59

| Proposed Specification | Acceptance Criteria | Lot 201698 | Lot 201699 | Lot 201700 | Lot 201731 |
|---|---|---|---|---|---|
| Appearance | White to off white free | Passes | Passes | Passes | Passes |
| | flowing powder | | | | |
| Identification (IR) | Compares to Standard | Passes | Passes | Passes | Passes |
| Assay (dry basis) | 98.0-101.0% | 100.2% | 100.4% | 99.0% | 98.9% |
| Moisture (% KF) | 4.5%-5.5% | 4.8% | 5.0% | 5.0% | 5.2% |
| Particle Size Analysis | | | | | |
| U.S. Standard #20 | NMT 1% On | 0% | 0% | 0% | 0% |
| U.S. Standard #40 | NMT 35% On | 15% | 20% | 19% | 20% |
| U.S. Standard #100 | NLT 18% On | 61% | 68% | 65% | 61% |
| U.S. Standard #200 | NMT 6% Thru | 5% | 2% | 3% | 4% |
| Tapped Density | 0.82 to 0.90 g/mL | 0.88 | 0.85 | 0.85 | 0.82 |
| Microbiology | | | | | |
| Total Plate Count | ≤1000 cfu/g | <10 | <10 | <10 | <10 |
| Yeast & Mold | ≤100 cfu/g | <10 | <10 | <10 | <10 |
| E. Coli | Negative | Passes | Passes | Passes | Passes |
| Salmonella | Negative | Passes | Passes | Passes | Passes |
| Pseudomonas A. | Negative | Passes | Passes | Passes | Passes |

Addition final product testing is shown in the following table.

TABLE 60

| Proposed Specification | Acceptance Criteria | Lot 201698 | Lot 201699 | Lot 201700 | Lot 201731 |
|---|---|---|---|---|---|
| Optical Rotation | +13.5° to +15.5° | +14.8 | +14.8 | +14.7 | +14.0 |
| Related Substances | Total − NMT 1.5% | 0.57% | 0.67% | 1.25% | 0.3% |
| Reducing Sugars | NMT 0.2% | Pass | Pass | Pass | Pass |
| Lead | NMT 0.5 ppm | Pass | Pass | Pass | Pass |
| Nickel | NMT 1 ppm | Pass | Pass | Pass | Pass |
| Heavy Metals | NMT 5 ppm | Pass | Pass | Pass | Pass |
| Sulfated Ash | NMT 0.1% | 0.0% | 0.0% | 0.0% | 0.0% |
| Appearance of Solution | No more intense than | Pass | Pass | Pass | Pass |
| | Reference Solution BY7 | | | | |
| Acidity or Alkalinity | NMT 0.2 mL 0.01M NaOH | Pass | Pass | Pass | Pass |
| | NMT 0.3 mL 0.01M HCl | | | | |

### Example 6

The following example provides tapped density accelerated stability testing results for Qualification lots (201698, 201699, and 201700) described in Example 4.

Testing conditions: 40° C. and 75% relative humidity; specification (0.82 to 0.90 g/mL)

TABLE 61

| Time | 201698 | 201699 | 201700 |
|---|---|---|---|
| (months) | (g/mL) | (g/mL) | (g/mL) |
| 0 | 0.88 | 0.85 | 0.85 |
| 1 | 0.84 | 0.84 | 0.87 |
| 2 | 0.87 | 0.87 | 0.88 |
| 3 | 0.88 | 0.88 | 0.90 |

These results are well within the specification limits for the 3 month study period and thus far predict greater than 12 months stability under controlled room temperature conditions.

### Example 7

This example provides initial 12 month stability data for lactitol granulates produced in accordance with the granulation method of the present invention. Three (3) qualification batches were analyzed after storage in 50-gallon drums under warehousing conditions.

The raw materials used in the production of the three lots were as follow:

TABLE 62

| Material | Item | | | | |
|---|---|---|---|---|---|
| Description | Number | % | mg/g | Manufacturer | Function |
| Lactitol | 9-5064 | 97.72 | 977.2 | Shandong Lujian | Active |
| Monohydrate | | | | Biological | ingredient |
| | | | | Technology/ | |
| | | | | Danisco | |
| Lactitol | 9-5067 | 2.28 | 22.8 | Shandong Lujian | Binder/ |
| Monohydrate | | | | Biological | Active |
| for Binder | | | | Technology/ | Ingredient |
| Solution | | | | Danisco | |
| Purified | 8-51 | ¹ | ¹ | PD | Vehicle for |
| Water, | | | | International | Binder |
| USP/Ph. Eur. | | | | | Solution |

Does not appear in finished product.

Each of three (3) lots were subjected to conditions of an accelerated stability study of 40° C. and 75% relative humidity (RH) for 6 months and a long term stability study under conditions of 25° C. and 60% RH for up to 36 months. Each lot was tested as shown in the following Table. All stability samples were packaged in fiber board mini drums that mimic the bulk packaging of the product.

TABLE 63
Storage Conditions and Test Frequencies

| Test | Storage Conditions | |
|---|---|---|
| Frequency | 25° C. ± 2° C. | 40° C. ± 2° C. |
| (Months) | 60% RH ± 5% | 75% RH ± 5% |
| 0 | X | — |
| 1 | — | X |
| 2 | — | X |
| 3 | X | X |
| 4 | — | X |
| 6 | X | X |
| 9 | X | — |
| 12 | X | — |
| 18 | X | — |
| 24 | X | — |
| 36 | X | — |

X = Mini Drum to be pulled at each this time point

Details of the stability testing requirements, storage conditions, and testing frequencies are included in the above Table and the following Table. The three lots were tested at all time-points listed in the above Table, according to the testing requirements listed in the following Table.

TABLE 64
Test Parameters

| Test Description | Specification | U/M | Method # |
|---|---|---|---|
| Appearance | White to off white | | 9-5065 |
| | free flowing | | |
| Assay | 98.0-101.0 | % | 9-5065 |
| Related | Total − NMT 1.5 | % | 9-5065 |
| Reducing Sugars | NMT 0.2 | % | 9-5065 |
| Tapped Density | 0.82-0.90 | g/mL | 9-5065 |
| Moisture (KF) | 4.5-5.5 | % | 9-5065 |

Six months accelerated (40° C./75% RH) and 12 months long-term (25° C./60% RH) stability studies of the granulates were completed as described above. Additionally, tapped density was tested at 8 and 12 months for full bulk drums stored at Controlled Room Temperature to assess the actual storage conditions for this product. This stability data is presented in the following Tables.

6-Month Accelerated (40° C./75% RH) Stability Study Results

TABLE 65
Accelerated Stability Assay (%)
Assay

| | | | | | 95% | | Assay | |
|---|---|---|---|---|---|---|---|---|
| Time | 201698 | 201699 | 201700 | Stdev | Confid | LCL | Avg (%) | UCL |
| 0 | 100.2 | 100.4 | 99.0 | 0.76 | 0.86 | 99.0 | 99.9 | 100.7 |
| 1 | 99.6 | 99.6 | 99.9 | 0.17 | 0.20 | 99.5 | 99.7 | 99.9 |
| 2 | 99.1 | 99.2 | 98.9 | 0.15 | 0.17 | 98.9 | 99.1 | 99.2 |
| 3 | 99.3 | 99.4 | 99.2 | 0.10 | 0.11 | 99.2 | 99.3 | 99.4 |
| 4 | 99.7 | 99.5 | 99.4 | 0.15 | 0.17 | 99.4 | 99.5 | 99.7 |
| 6 | 98.9 | 99.5 | 99.5 | 0.35 | 0.39 | 98.9 | 99.3 | 99.7 |

| | | | | | | | |
|---|---|---|---|---|---|---|---|
| Slope | −0.153 | −0.103 | 0.036 | Slope | −0.024 | −0.073 | −0.123 |
| Intercept | 99.9 | 99.9 | 99.2 | Intercept | 99.2 | 99.7 | 100.1 |
| Crossing Spec at | 98.0 | 98.0 | 101.0 | Crossing Spec at | 98.0 | 98.0 | 98.0 |
| Forecast(M) | 12.3 | 18.2 | 49.8 | Forecast(M) | 50.4 | 22.6 | 17.2 |

UCL Intercept at 98.0% at 17.2 Months

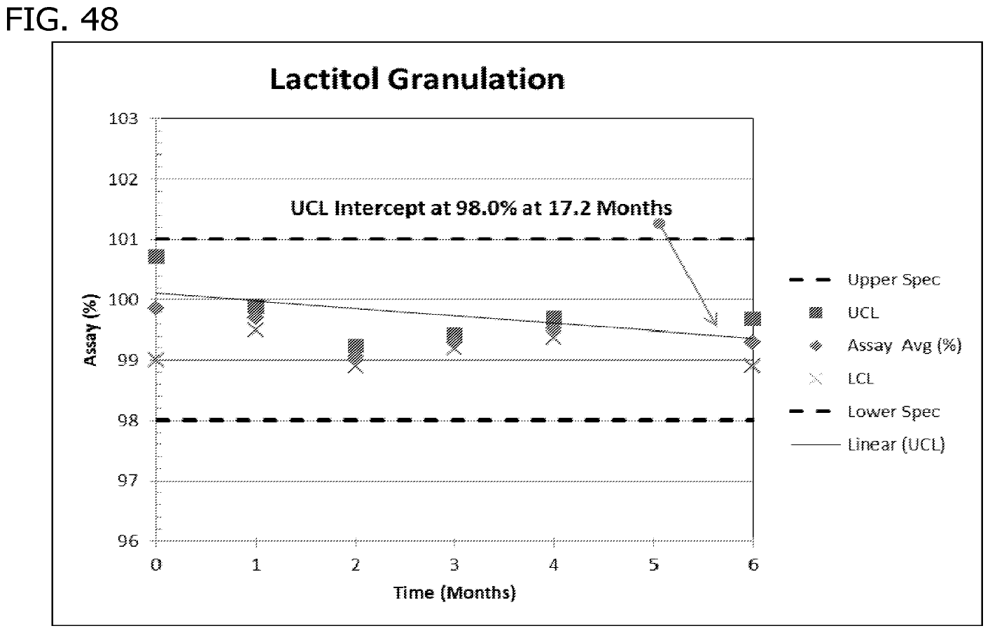

As shown in FIG. 48, the accelerated study assay trend line at the upper-confidence-level (UCL) predicts that the product will remain within the specification of 98.0% to 101.0% for over 17 months at 40° C./75% RH. The 17 month prediction at 40° C./75% RH is equivalent to 68 months at RT, which is well over the 6 month bulk product shelf-life assignment. The average assay remains at 99.1-99.9% over the 6-months of the accelerated stability study.

TABLE 66
Accelerated Stability Moisture
Moisture

| | | | | | 95% | | Moisture | |
|---|---|---|---|---|---|---|---|---|
| Time | 201698 | 201699 | 201700 | Stdev | Confid | LCL | Avg (%) | UCL |
| 0 | 4.8 | 5.0 | 5.0 | 0.12 | 0.13 | 4.8 | 4.9 | 5.1 |
| 1 | 5.1 | 5.1 | 5.1 | 0.00 | 0.00 | 5.1 | 5.1 | 5.1 |
| 2 | 5.0 | 5.0 | 5.0 | 0.00 | 0.00 | 5.0 | 5.0 | 5.0 |
| 3 | 5.1 | 5.1 | 5.3 | 0.12 | 0.13 | 5.0 | 5.2 | 5.3 |
| 4 | 5.1 | 5.1 | 5.1 | 0.00 | 0.00 | 5.1 | 5.1 | 5.1 |
| 6 | 5.0 | 5.0 | 5.1 | 0.06 | 0.07 | 5.0 | 5.0 | 5.1 |

| | | | | | | | |
|---|---|---|---|---|---|---|---|
| Slope | 0.023 | 0.000 | 0.017 | Slope | 0.017 | 0.013 | 0.010 |
| Intercept | 4.96 | 5.05 | 5.05 | Intercept | 5.0 | 5.0 | 5.1 |
| Crossing Spec at | 5.5 | 5.5 | 5.5 | Crossing Spec at | 5.5 | 5.5 | 5.5 |
| Forecast(M) | 23.8 | — | 26.0 | Forecast(M) | 31.9 | 36.0 | 43.4 |

LCL Intercept at 5.5% at 31.9 Months

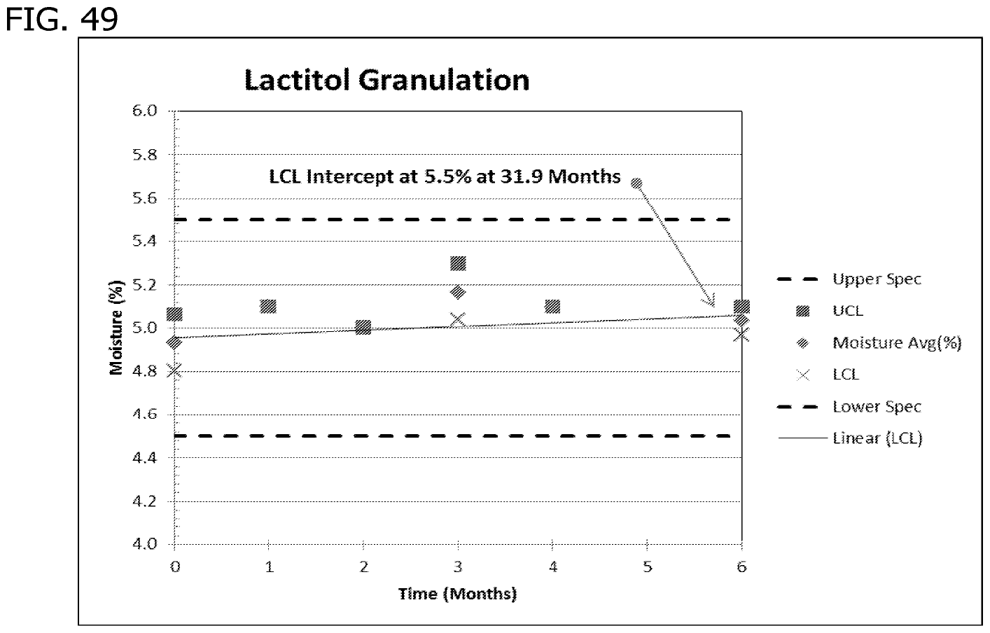

As shown in FIG. 49, the accelerated study moisture trend line at the lower-confidence-level (LCL) predicts that the product will remain within specification for over 31 months at 40° C./75% RH. The 31 month prediction at 40° C./75% RH is equivalent to 124 months at RT, which is well over the 6 month bulk product shelf-life assignment. The average moisture remains at 4.9%-5.1% over the 6-months of the accelerated stability study.

TABLE 67
Accelerated Stability Related Substances
Related Substances (%)

| | | | | | 95 % | | Related | |
|---|---|---|---|---|---|---|---|---|
| Time | 201698 | 201699 | 201700 | Stdev | Confid | LCL | Sub | UCL |
| 0 | 0.57 | 0.67 | 0.28 | 0.20 | 0.23 | 0.28 | 0.51 | 0.74 |
| 1 | 0.25 | 0.26 | 0.25 | 0.01 | 0.01 | 0.25 | 0.25 | 0.26 |
| 2 | 0.50 | 0.50 | 0.40 | 0.06 | 0.07 | 0.40 | 0.47 | 0.53 |
| 3 | 0.30 | 0.30 | 0.30 | 0.00 | 0.00 | 0.30 | 0.30 | 0.30 |
| 4 | 0.30 | 0.30 | 0.30 | 0.00 | 0.00 | 0.30 | 0.30 | 0.30 |
| 6 | 0.45 | 0.45 | 0.38 | 0.04 | 0.05 | 0.38 | 0.43 | 0.47 |

| | | | | | | | |
|---|---|---|---|---|---|---|---|
| Slope | −0.012 | −0.024 | 0.015 | Slope | 0.015 | −0.007 | −0.029 |
| Intercept | 0.4 | 0.5 | 0.3 | Intercept | 0.3 | 0.4 | 0.5 |
| Crossing Spec at | 1.5 | 1.5 | 1.5 | Crossing Spec at | 1.5 | 1.5 | 1.5 |
| Forecast(M) | -92.8 | -43.2 | 84.1 | Forecast(M) | 81.4 | −159.6 | −34.3 |

LCL Intercept at 1.5 at 81.4 Months

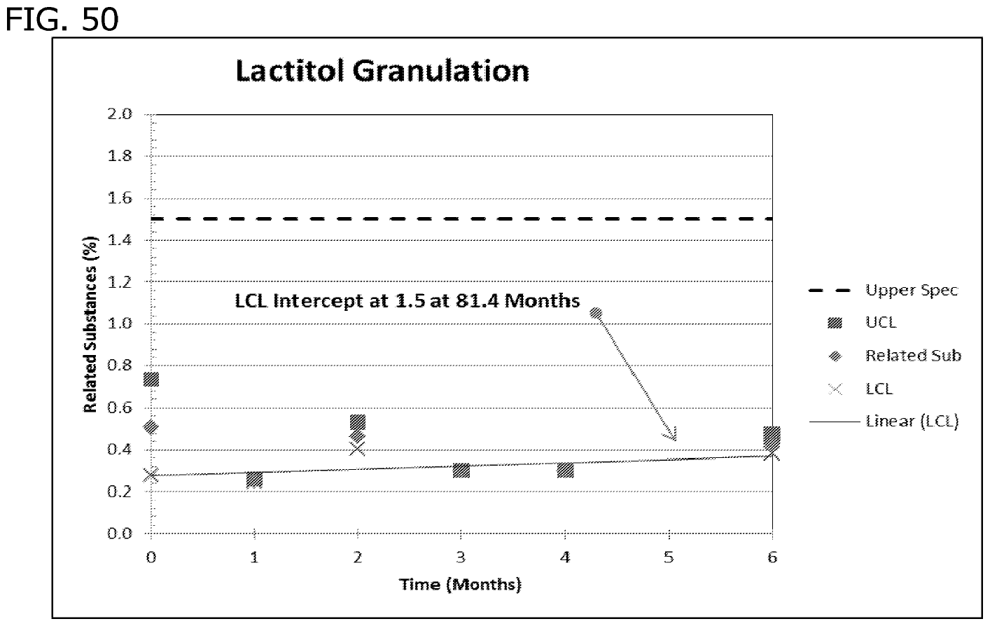

As shown in FIG. 50, the accelerated study related substances trend line at the lower-confidence-level (LCL) predicts that the product will remain within specification of NMT 1.5% for over 81 months at 40° C./75% RH. The 81 month prediction at 40° C./75% RH is equivalent to 324 months at RT, which is well over the 6 month bulk product shelf-life assignment. The average related substances remain at 0.30%-0.51% over the 6-months of the accelerated stability study.

TABLE 68
Accelerated Stability Tapped Density

| | | | | | 95% | | Tap | |
|---|---|---|---|---|---|---|---|---|
| Time (months) | 201698 | 201699 | 201700 | Stdev | Confid | LCL | Density | UCL |
| 0 | 0.88 | 0.85 | 0.85 | 0.02 | 0.02 | 0.84 | 0.86 | 0.88 |
| 1 | 0.84 | 0.84 | 0.87 | 0.02 | 0.02 | 0.83 | 0.85 | 0.87 |
| 2 | 0.87 | 0.87 | 0.88 | 0.01 | 0.01 | 0.87 | 0.87 | 0.88 |
| 3 | 0.88 | 0.88 | 0.90 | 0.01 | 0.01 | 0.87 | 0.89 | 0.90 |
| 4 | 0.91 | 0.98 | 0.91 | 0.04 | 0.05 | 0.89 | 0.93 | 0.98 |
| 6 | 0.90 | 0.90 | 0.92 | 0.01 | 0.01 | 0.89 | 0.91 | 0.92 |

| | | | | | | | |
|---|---|---|---|---|---|---|---|
| Slope | 0.008 | 0.015 | 0.012 | Slope | 0.011 | 0.012 | 0.012 |
| Intercept | 0.9 | 0.8 | 0.9 | Intercept | 0.8 | 0.9 | 0.9 |
| Crossing Spec at | 0.90 | 0.90 | 0.90 | Crossing Spec at | 0.90 | 0.90 | 0.90 |
| Forecast(M) | 5.9 | 3.9 | 4.1 | Forecast(M) | 6.3 | 4.4 | 2.7 |

UCL Intercept at 0.90% at 2.7 Months

[symbol]

results [symbol]
indicates data missing or illegible when filed

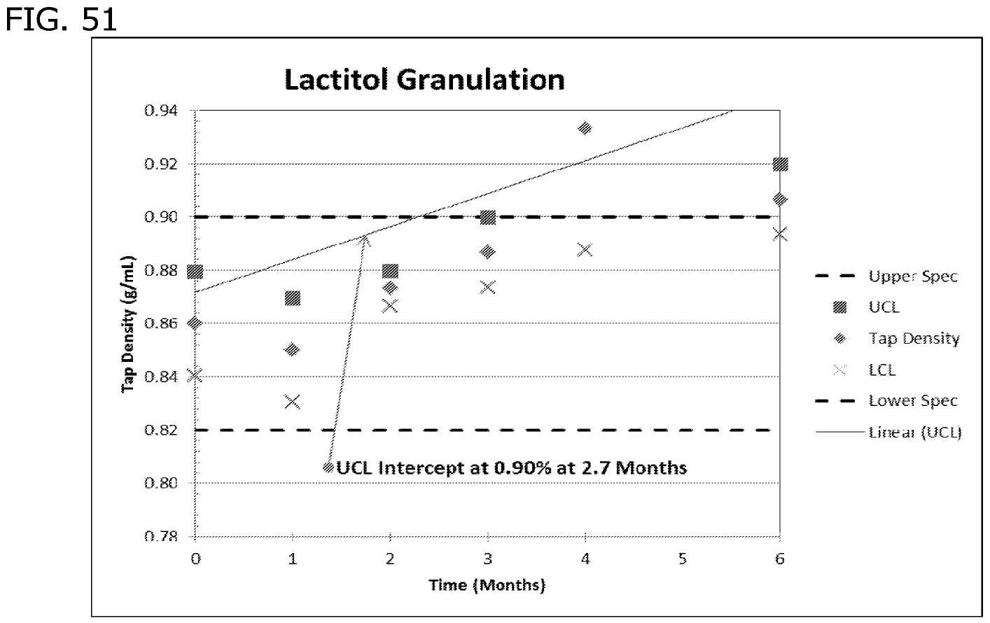

As shown in FIG. 51, the accelerated study tapped density trend line at the upper-confidence-level (UCL) predicts that the product will remain within one possible specification of 0.82 to 0.90 g/mL for 2.7 months at 40° C./75% RH. The 2.7 month prediction at 40° C./75% RH is equivalent to 10.8 months at RT, which is over the 6 month bulk product shelf-life assignment. The average tapped density ranges from 0.85 to 0.93 over the 6-months of the accelerated stability study. Tapped density results for all three (3) lots at 4 months and one lot at 6 months were out-of-specification with results above 0.90. This may suggest that the bulk packaged granulated powder should undergo final product packaging within 10 months, which is within standard pharmaceutical practices.

In addition to the quantitative tests discussed above, results for two Pass/Fail tests are shown in the following Table.

TABLE 69
Accelerated Stability Other Tests

| | Appearance | | | | | |
|---|---|---|---|---|---|---|
| | White to off white | | | Reducing Sugars | | |
| 40° C./75% RH | free flowing powder | | | NMT 0.2% | | |
| Time (month) | 201698 | 201699 | 201700 | 201698 | 201699 | 201700 |
| 0 | Pass | Pass | Pass | Pass | Pass | Pass |
| 1 | Pass | Pass | Pass | Pass | Pass | Pass |
| 2 | Pass | Pass | Pass | Pass | Pass | Pass |
| 3 | Pass | Pass | Pass | Pass | Pass | Pass |
| 4 | Pass | Pass | Pass | Pass | Pass | Pass |
| 6 | Pass | Pass | Pass | Pass | Pass | Pass |

All other stability test results met specification at all time-points tested.

Summary: 6-Month Accelerated (40° C./75% RH) Stability Study

The accelerated stability data for six months from these studies are well within the specifications for the full 6-months, with the exception of the tapped density, for all test parameters and would support a 24 month product expiration dating. The tapped density trends upward over time at accelerated conditions (40° C./75% RH) with the tapped density going out of specification at 4-months. All lots pass at 3-months (suggesting 12-month RT stability) but the variation seen in the tapped density test caused the upper-confidence-level (UCL) trend line to predict a bulk product shelf life of ˜10.8 months.

12-Month Long Term (25° C./60% RH) Stability Study Results

The Long Term stability data for up to 12 months from these studies (shown below) are well within the specifications and are provided herein.

TABLE 70
Long Term (12-month) Stability Assay (% of LC)
Assay

| | | | | | 95 % | | Assay | |
|---|---|---|---|---|---|---|---|---|
| Time | 201698 | 201699 | 201700 | Stdev | Confid | LCL | Avg (%) | UCL |
| 0 | 100.2 | 100.4 | 99.0 | 0.76 | 0.86 | 99.0 | 99.9 | 100.7 |
| 3 | 99.2 | 99.2 | 99.3 | 0.06 | 0.07 | 99.2 | 99.2 | 99.3 |
| 6 | 99.3 | 99.5 | 99.5 | 0.12 | 0.13 | 99.3 | 99.4 | 99.6 |
| 9 | 98.5 | 99.0 | 98.8 | 0.25 | 0.28 | 98.5 | 98.8 | 99.1 |
| 12 | 99.5 | 99.6 | 99.9 | 0.21 | 0.24 | 99.4 | 99.7 | 99.9 |
| 18 | | | | | | | | |

| | | | | | | | |
|---|---|---|---|---|---|---|---|
| Slope | −0.070 | −0.060 | 0.043 | Slope | 0.005 | −0.029 | −0.063 |
| Intercept | 99.8 | 99.9 | 99.0 | Intercept | 99.0 | 99.6 | 100.1 |
| Crossing Spec at | 98.0 | 98.0 | 101.0 | Crossing Spec at | 101.0 | 98.0 | 98.0 |
| Forecast(M) | 25.1 | 31.7 | 45.2 | Forecast(M) | 374.5 | 54.2 | 33.1 |

UCL Intercept at 98.0% at 33.1 Months

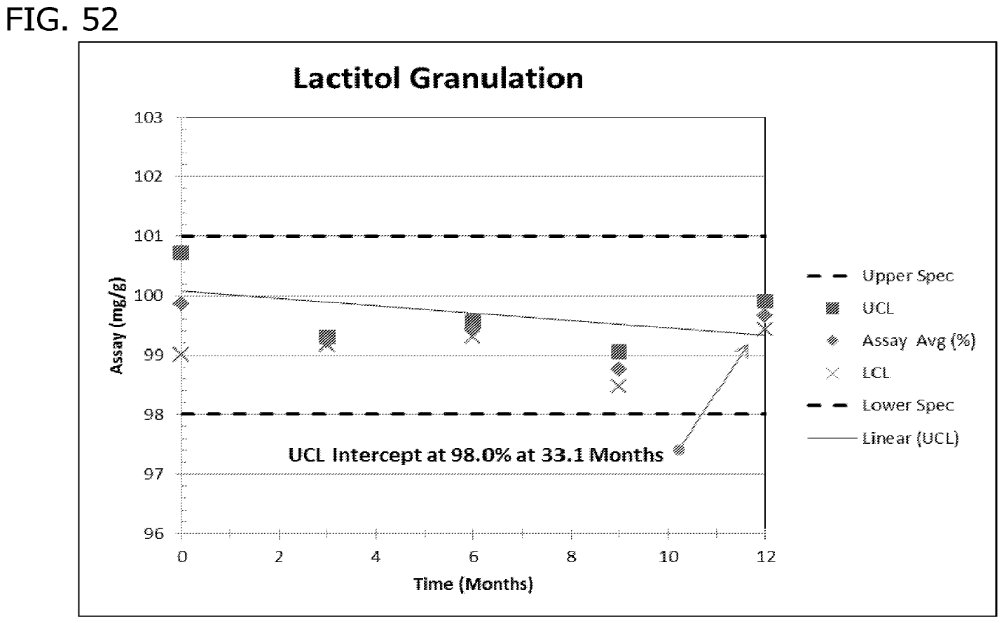

As shown in FIG. 52, the long term study assay trend line at the upper-confidence-level (UCL) predicts a bulk product shelf-life of over 33 months at 25° C./60% RH, well over the 6 month bulk product shelf-life assignment. The average assay remains at 99.2-99.9% over the 12-months of the 25° C./60% RH stability study.

TABLE 71
Long Term (12-month) Stability Moisture
Moisture

| | | | | | 95 % | | Moisture | |
|---|---|---|---|---|---|---|---|---|
| Time | 201698 | 201699 | 201700 | Stdev | Confid | LCL | Avg (%) | UCL |
| 0 | 4.8 | 5.0 | 5.0 | 0.12 | 0.13 | 4.8 | 4.9 | 5.1 |
| 3 | 5.3 | 5.2 | 5.3 | 0.06 | 0.07 | 5.2 | 5.3 | 5.3 |
| 6 | 5.1 | 5.1 | 5.1 | 0.00 | 0.00 | 5.1 | 5.1 | 5.1 |
| 9 | 5.3 | 5.1 | 5.3 | 0.12 | 0.13 | 5.1 | 5.2 | 5.4 |
| 12 | 5.1 | 5.0 | 5.1 | 0.06 | 0.07 | 5.0 | 5.1 | 5.1 |
| 18 | | | | | | | | |

| | | | | | | | |
|---|---|---|---|---|---|---|---|
| Slope | 0.020 | −0.003 | 0.007 | Slope | 0.010 | 0.008 | 0.006 |
| Intercept | 5.00 | 5.10 | 5.12 | Intercept | 5.0 | 5.1 | 5.2 |
| Crossing Spec at | 5.5 | 4.5 | 5.5 | Crossing Spec at | 5.5 | 5.5 | 5.5 |
| Forecast(M) | 25.0 | 180.0 | 57.0 | Forecast(M) | 52.1 | 54.8 | 59.8 |

LCL Intercept at 5.5% at 52.1 Months

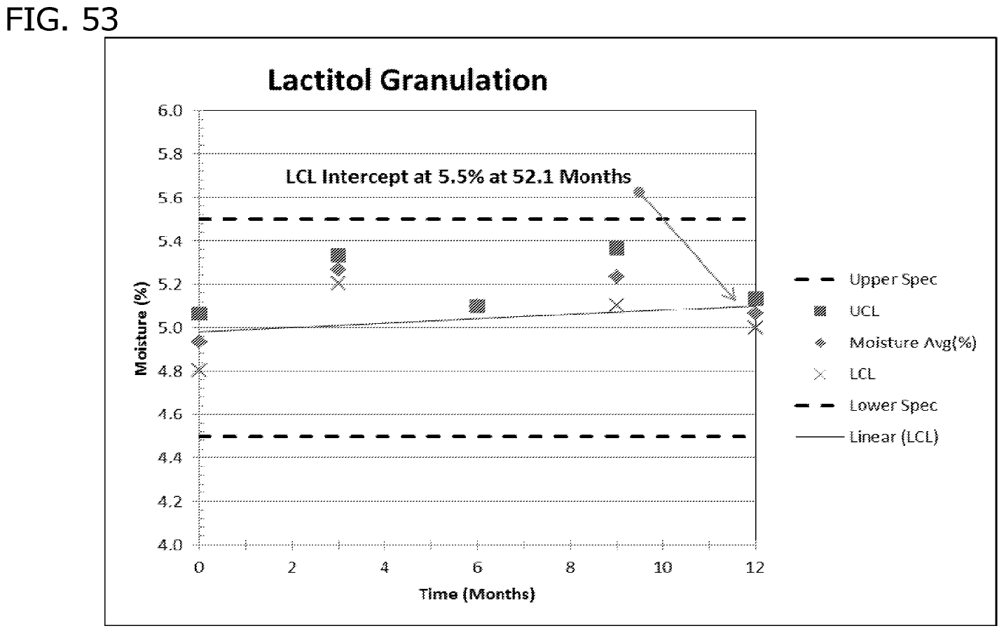

As shown in FIG. 53, the long term study moisture trend line at the lower-confidence-level (LCL) predicts a bulk product shelf-life of over 52 months at 25° C./60% RH, well over the 6 month bulk product shelf-life assignment. The average moisture remains between 4.9%-5.3% over the 12-months of the 25° C./60% RH stability study.

TABLE 72
Long Term (12-month) Stability Related Substance
Related Substances (%)

| | | | | | 95 % | | Related | |
|---|---|---|---|---|---|---|---|---|
| Time | 201698 | 201699 | 201700 | Stdev | Confid | LCL | Sub | UCL |
| 0 | 0.57 | 0.67 | 0.28 | 0.20 | 0.23 | 0.3 | 0.5 | 0.7 |
| 3 | 0.26 | 0.28 | 0.21 | 0.04 | 0.04 | 0.2 | 0.3 | 0.3 |
| 6 | 0.33 | 0.37 | 0.42 | 0.05 | 0.05 | 0.3 | 0.4 | 0.4 |
| 9 | 0.28 | 0.30 | 0.37 | 0.05 | 0.05 | 0.3 | 0.3 | 0.4 |
| 12 | 0.38 | 0.41 | 0.36 | 0.03 | 0.03 | 0.4 | 0.4 | 0.4 |
| 18 | | | | | | | | |

| | | | | | | | |
|---|---|---|---|---|---|---|---|
| Slope | −0.012 | −0.017 | 0.011 | Slope | 0.007 | −0.006 | −0.019 |
| Intercept | 0.4 | 0.5 | 0.3 | Intercept | 0.2 | 0.4 | 0.6 |
| Crossing Spec at | 1.5 | 1.5 | 1.5 | Crossing Spec at | 1.5 | 1.5 | 1.5 |
| Forecast(M) | −88.7 | −59.6 | 115.9 | Forecast(M) | 180.5 | −183.0 | −49.6 |

LCL Intercept at 1.5 at 180.5 Months

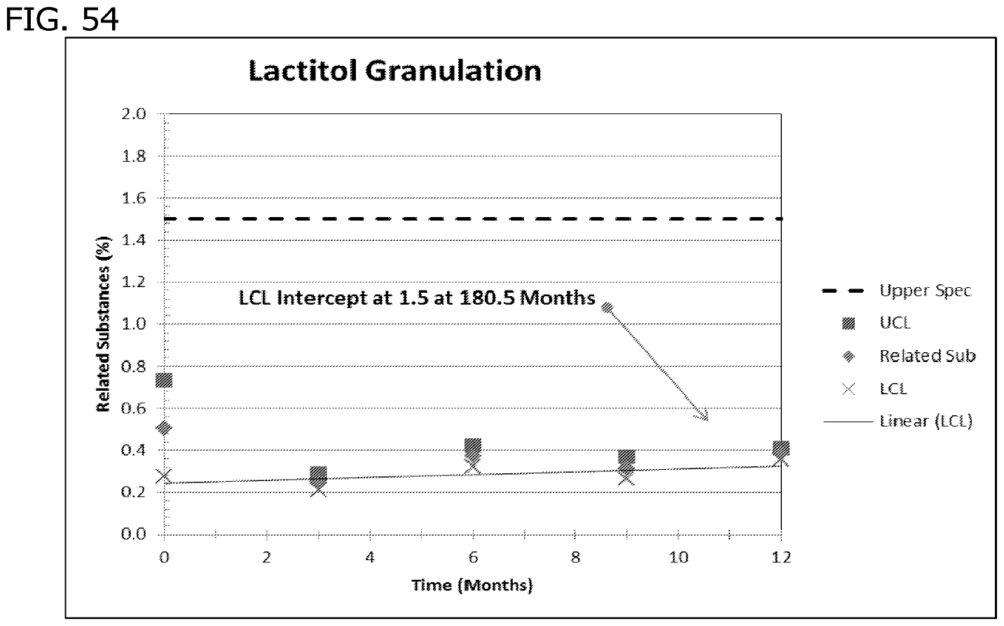

As shown in FIG. 54, the long term study related substances trend line at the lower-confidence-level (LCL) predicts a bulk product shelf-life of over 180 months at 25° C./60% RH, well over the 6 month bulk product shelf-life assignment. The average related substances remain between 0.3%-0.5% over the 12-months of the 25° C./60% RH stability study. The products were stored under standard, internationally agreed upon storage conditions for testing drug stability (The European Agency for the Evaluation of Medicinal Products Human Medicines Evaluation Unit; CPMP/ICH/380/95).

TABLE 73
Long Term (12-month) Stability Tapped Density
Tap Density (g/mL)

| | | | | | 95 % | | Tap | |
|---|---|---|---|---|---|---|---|---|
| Time (months) | 201698 | 201699 | 201700 | Stdev | Confid | LCL | Density | UCL |
| 0 | 0.88 | 0.85 | 0.85 | 0.02 | 0.02 | 0.84 | 0.86 | 0.88 |
| 3 | 0.86 | 0.83 | 0.87 | 0.02 | 0.02 | 0.83 | 0.85 | 0.88 |
| 6 | 0.90 | 0.87 | 0.90 | 0.02 | 0.02 | 0.87 | 0.89 | 0.91 |
| 9 | 0.90 | 0.88 | 0.89 | 0.01 | 0.01 | 0.88 | 0.89 | 0.90 |
| 12 | 0.90 | 0.88 | 0.89 | 0.01 | 0.01 | 0.88 | 0.89 | 0.90 |
| 18 | | | | | | | | |

| | | | | | | | |
|---|---|---|---|---|---|---|---|
| Slope | 0.003 | 0.004 | 0.003 | Slope | 0.004 | 0.003 | 0.002 |
| Intercept | 0.9 | 0.8 | 0.9 | Intercept | 0.8 | 0.9 | 0.9 |
| Crossing Spec at | 0.90 | 0.90 | 0.90 | Crossing Spec at | 0.90 | 0.90 | 0.90 |
| Forecast(M) | 12.3 | 17.7 | 13.5 | Forecast(M) | 16.8 | 14.8 | 10.9 |

UCL Intercept at 0.90% at 10.9 Months

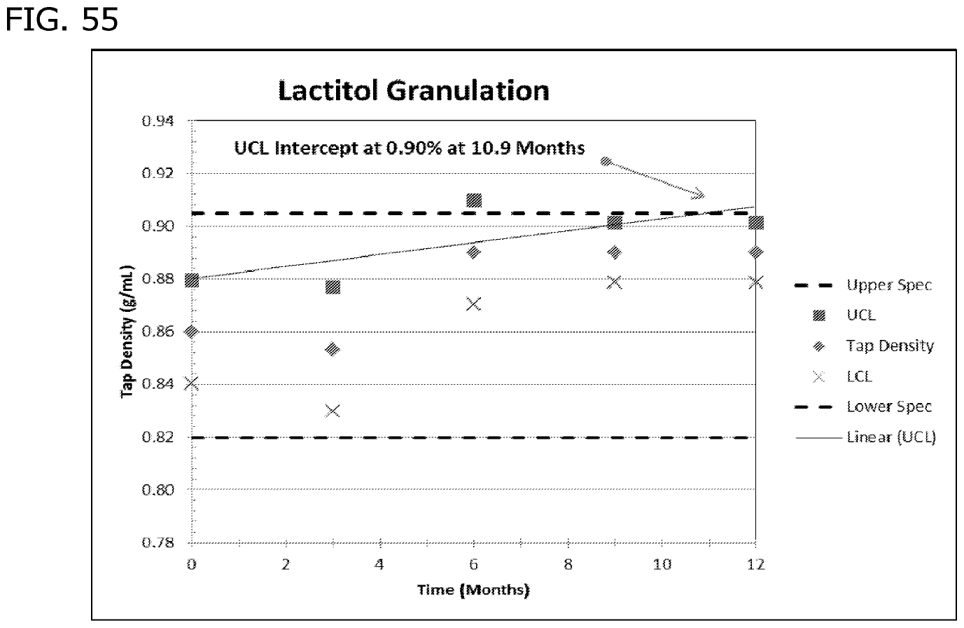

As shown in FIG. 55, the long term study tapped density trend line at the upper-confidence-level (UCL) predicts a bulk product shelf-life of over 10 months at 25° C./60% RH, in line with the 6 month bulk product shelf-life assignment. The average tapped density remains between 0.85-0.89 over the 12-months of the 25° C./60% RH stability study.

Full bulk drug drums were stored at Controlled Room Temperature conditions (20° C.−25° C.) and tested for tapped density after 12 months. The tapped density results for the three stability lots stored at controlled room temperature stability conditions (shown below) are well within the specifications at 12 months.

TABLE 74
Controlled Room Temperature Stability Tapped Density
Tap Density (g/mL)

| | | | | | 95 % | | Tap | |
|---|---|---|---|---|---|---|---|---|
| Time (months) | 201698 | 201699 | 201700 | Stdev | Confid | LCL | Density | UCL |
| 0 | 0.88 | 0.85 | 0.85 | 0.02 | 0.02 | 0.84 | 0.86 | 0.88 |
| 8 | 0.88 | 0.83 | 0.88 | 0.03 | 0.03 | 0.83 | 0.86 | 0.89 |
| 12 | 0.85 | 0.83 | 0.86 | 0.01 | 0.02 | 0.83 | 0.85 | 0.86 |
| 18 | | | | | | | | |
| 24 | | | | | | | | |

| | | | | | | | |
|---|---|---|---|---|---|---|---|
| Slope | −0.0022 | −0.0015 | 0.0012 | Slope | −0.0007 | −0.0008 | −0.0010 |
| Intercept | 0.9 | 0.8 | 0.9 | Intercept | 0.8 | 0.9 | 0.9 |
| Crossing Spec at | 0.82 | 0.82 | 0.90 | Crossing Spec at | 0.82 | 0.82 | 0.82 |
| Forecast(M) | 29.0 | 19.4 | 38.0 | Forecast(M) | 29.7 | 51.1 | 66.0 |

LCL Intercept at 0.82% at 29.7 Months

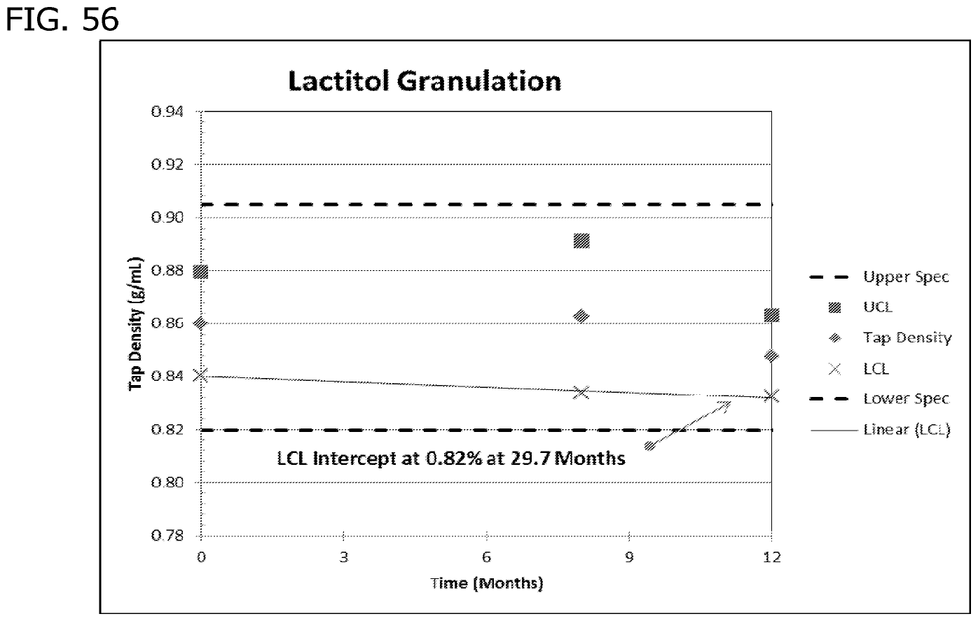

A shown in FIG. 56, the Controlled Room Temperature study tapped density trend line at the upper-confidence-level (UCL) predicts a bulk product shelf-life of over 29 months, in line with the 6 month bulk product shelf-life assignment. Other than the expected ±0.02 g/mL variation in the tap density due to the method's limitation of volume measurement, there is no change in tap density at the true storage conditions of the product.

Other Test Results

In addition to the quantitative tests discussed above, results for two Pass/Fail tests are shown in the following Table.

TABLE 75
Long Term Stability Other Tests

| | Appearance | | | | | |
|---|---|---|---|---|---|---|
| | White to off white | | | Reducing Sugars | | |
| 25° C./60% RH | free flowing powder | | | NMT 0.2% | | |
| Time (month) | 201698 | 201699 | 201700 | 201698 | 201699 | 201700 |
| 0 | Pass | Pass | Pass | Pass | Pass | Pass |
| 3 | Pass | Pass | Pass | Pass | Pass | Pass |
| 6 | Pass | Pass | Pass | Pass | Pass | Pass |
| 9 | Pass | Pass | Pass | Pass | Pass | Pass |
| 12 | Pass | Pass | Pass | Pass | Pass | Pass |

All other stability test results met specification at all time-points tested.

Summary: Long Term (25° C./60% RH) and Controlled Room Temperature Stability Study

The long term stability data for twelve months from these studies are well within the specifications for the full 12-months for all test parameters and would support a 24 month product expiration dating. The tapped density trends upward over time but remains within specification at 6-months. All lots pass at 12-months (supporting greater than 6-month RT stability), but due to the variation seen in the test, the upper-confidence-level (UCL) trend line predicts a bulk product shelf life of 10.9 months. This may suggest that the bulk packaged granulated powder should undergo final product packaging within 10-months which is within standard pharmaceutical practices. Lactitol stored in the full bulk drums stored at Controlled Room Temperature to assess the true storage conditions for this product had trend lines predicting bulk product shelf-life of over 29 months. In the true bulk packaging configuration, only a slight downward trend in the tap density can be seen at 12-months.

Interim Stability Conclusion

All accelerated stability results are well within specification limits for the 6 month study period, except for the tapped density which goes out of specification at 4 months. Trends for assay, moisture, and related substances from the accelerated stability studies all predict greater than 24 months stability. Trends for tapped density predict a bulk product shelf life of ˜10 months.

All long term (25° C./60% RH) stability results are well within specification limits for the 12 month study period. Trends for assay, moisture, and related substances from the long term stability studies all predict greater than 24 months stability. Trends for tapped density predict a bulk product shelf life of ˜10.9 months, while in the true bulk packaging configuration, only a slight downward trend in the tap density can be seen at 12-months.

These data support an expiration date of 6 months for the three lots of granulates subjected to the above tests.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above without departing from the scope of the invention, it is intended that all matter contained in the above description and shown in the accompanying drawing[s] shall be interpreted as illustrative and not in a limiting sense.

1. A process for preparing a lactitol granulate, the process comprising:
introducing a binder solution comprising lactitol and an aqueous solution into a high shear granulation apparatus comprising one or more mixing zones;
introducing particulate lactitol into the high shear granulation apparatus;
combining the binder solution and particulate lactitol within a mixing zone of the high shear granulation apparatus, thereby forming a particulate lactitol mass; and
drying the particulate lactitol mass, thereby forming the lactitol granulate, wherein drying the particulate lactitol mass comprises:
introducing the particulate mass into a first drying zone, the temperature within the first drying zone being below 40° C.; and
following removal from the first drying zone introducing the particulate mass into a second drying zone, the temperature within the second drying zone being above 40° C.

2. The process of claim 1 wherein lactitol constitutes at least about 20 weight % of the binder solution.

3. (canceled)

4. (canceled)

5. (canceled)

6. (canceled)

7. The process of claim 1 wherein the lactitol granulate satisfies the EU-NP and USP-NF definitions of lactitol monohydrate.

8. (canceled)

9. (canceled)

10. (canceled)

11. The process of claim 1 wherein the temperature within the first drying zone is below the glass transition temperature (Tg) of lactitol.

12. (canceled)

13. (canceled)

14. (canceled)

15. (canceled)

16. (canceled)

17. (canceled)

18. (canceled)

19. (canceled)

20. The process of claim 1 wherein the particulate lactitol is subjected to a size reduction unit operation prior to introduction into the high shear granulation apparatus.

21. The process of claim 20 wherein the particulate lactitol is subjected to size reduction unit operation separated from the group consisting of jet milling or cryogenic milling.

22. (canceled)

23. (canceled)

24. The process of claim 1, the process further comprising removing a portion of the particulate mass from the high shear granulation apparatus and combining this portion with the particulate lactitol prior to its introduction into the high shear granulation apparatus.

25. (canceled)

26. The process of claim 1 wherein the binder solution consists essentially of lactitol and water.

27. (canceled)

28. (canceled)

29. (canceled)

30. (canceled)

31. (canceled)

32. (canceled)

33. (canceled)

34. The process of claim 1
wherein the ratio of the mass flow rate of the particulate lactitol into the high shear granulation apparatus to the mass flow rate of the binder solution into the high shear granulation apparatus is at least 10:1.

35. The process of claim 1,
wherein the mass flow rate of the binder solution into the high shear granulation is controlled to provide a moisture content of the particulate lactitol mass of from about 5 wt % to about 10 wt %.

36. The process of claim 1
wherein:
prior to introduction into the high shear granulation apparatus, at least a portion of the particulate lactitol is subjected to a milling operation selected from the group consisting of jet milling or cryogenic milling.

37. The process of claim 36 wherein the milling operation does not subject the particulate lactitol to a temperature above its glass transition temperature.

38. The process of claim 37, the process further comprising drying the particulate lactitol mass, wherein drying comprises:
introducing the particulate mass into a first drying zone; and
following removal of the particulate mass from the first drying zone introducing the particulate mass into a second drying zone.

39. The process of claim 38 wherein the temperature within the first drying zone is at least 10° C. less than the temperature within the second drying zone.

40. (canceled)

41. (canceled)

42. (canceled)

43. (canceled)

44. A particulate lactitol granulate, wherein the granulate is prepared by the process of claim 1.

45. A particulate lactitol granulate composition, the composition comprising a lactitol granulate, wherein the granulate exhibits a tapped density of from about 0.8 g/mL to about 0.9 g/mL.

46. A particulate lactitol granulate composition, wherein:
the granulate exhibits a tapped density of at least about 0.75 g/mL; and
the composition exhibits a viscosity (flowability) of from about 6000 centipoise (cps) to about 7500 cps.

47. A particulate lactitol granulate composition, wherein the composition has a particle size distribution such that:
a. no more than 1 wt % of the composition is retained on a #20 US Standard Sieve;
b. no more than 35 wt % of the composition is retained on a #40 US Standard Sieve;
c. at least 15 wt % of the composition is retained on a #100 US Standard Sieve;
d. no more than 10 wt % of the composition passes through a #200 US Standard Sieve.

48. A fast-dissolving particulate lactitol granulate composition, wherein the composition exhibits a dissolution rate of less than 40 seconds when subjected to laser light scattering analysis.

49. (canceled)

50. (canceled)

51. (canceled)

52. (canceled)

53. (canceled)

54. (canceled)

55. (canceled)

56. (canceled)

57. A method for the treatment of constipation, the method comprising administering to a subject in need thereof a composition comprising the particulate lactitol granulate composition of any of the preceding claims.

58. (canceled)

59. (canceled)
